(19) United States
(12) Patent Application Publication
(10) Pub. No.: US 2022/0296866 A1
(43) Pub. Date: Sep. 22, 2022

(54) METHODS, DEVICES AND COMPOSITIONS FOR LOCAL DELIVERY

(71) Applicant: POLY-MED, INC., Anderson, SC (US)

(72) Inventors: Michael Scott Taylor, Anderson, SC (US); David Gravett, Mountain View, CA (US); Kara Bethany Acampora, West Union, SC (US); David Shalaby, Fair Play, SC (US); Joel Thomas Corbett, Anderson, SC (US)

(21) Appl. No.: 17/617,909

(22) PCT Filed: Jun. 10, 2020
(86) PCT No.: PCT/US20/37075

§ 371 Date: Dec. 9, 2021

Related U.S. Application Data
(60) Provisional application No. 62/859,555, filed on Jun. 10, 2019.

(51) Int. Cl. A61M 31/00; A61L 31/16; A61K 9/00; A61L 17/10

(52) U.S. Cl. CPC A61M 31/002; A61L 31/16; A61K 9/0034; A61K 9/0024; A61L 17/105; A61F 2210/0004; A61F 2250/0068

(57) ABSTRACT

Disclosed herein are methods, devices and compositions for degradable medical devices and/or compositions that provide local and/or systemic delivery of at least one active agent.

### CROSS-REFERENCE TO RELATED APPLICATION

This application claims the benefit under 35 U.S.C. § 119(e) of U.S. Provisional Patent Application No. 62/859,555, entitled Methods, Devices and Compositions for Local Delivery of Active Agent to Bladder, filed Jun. 10, 2019, which is herein incorporated in its entirety for all purposes.

### FIELD OF THE DISCLOSURE

The present disclosure relates generally to biodegradable medical devices and/or compositions for local delivery of active agents, particularly for local delivery to a urinary bladder.

### BACKGROUND

Some nonresorbable medical devices are implanted (i.e., placed on or within skin, tissue, a structure or an organ) in a subject permanently or for a limited period of time. A nonresorbable device may be left in place indefinitely, which is satisfactory in cases where the long-term presence of the device is not harmful and may be necessary for the particular treatment. Other nonresorbable devices are in place for a limited period of time and then may be physically removed, although that often requires additional medical intervention. As an alternative, a device may be formed at least in part from a bioabsorbable material that will degrade and/or absorb within the subject and the degradation products and/or metabolites thereof may be eventually excreted, preferably without further intervention by medical practitioners. Bioabsorable devices are increasingly desired by health care providers, however such devices may cause undesired results, e.g., the subject does not tolerate the degradation products.

There is a need in the art for medical devices and/or compositions for implantation into a subject that do not cause harm to the subject as they degrade, or at the very least cause reduced harm to the subject as they degrade. The present invention is directed to fulfilling that need.

### BRIEF DESCRIPTION OF THE DRAWINGS

FIG. 1 shows exemplary active agent-delivering medical devices in a body cavity.

FIG. 2 is a graph showing exemplary results from an erdafitinib release study at 37° C. in artificial urine.

FIG. 3 is a graph showing exemplary results from erdafitinib release study at 37° C. in artificial urine (micrograms erdafitinib released per day)

FIG. 4 is a graph showing exemplary results from an erdafitinib release study at 37° C. in artificial urine (micrograms erdafitinib released per gram of drug containing formulation per day)

FIG. 5 is a graph showing exemplary results of an active agent release study.

### SUMMARY

The present disclosure comprises active agent-delivering degradable medical devices and/or compositions which may comprise degradable compositions. In an aspect, an active agent-delivering medical devices and/or compositions comprises components for controlled delivery of pharmaceutical agents locally to a body cavity, such as in the bladder. For example, local controlled-delivery may comprise whole-organ delivery via release from within a body cavity, or from within the organ wall which forms the cavity, and/or localized delivery to a specific body cavity and/or to specific areas within a body cavity, e.g. for intratumoral delivery of a chemotherapeutic agent. As used herein, “degradable” includes resorption of all or a portion of a device or composition and or includes disintegration or physical disruption of all or a portion of a device or composition.

The present disclosure may include active agent-delivering degradable medical devices and/or compositions comprising one or more of a pharmaceutically active agent, a depot carrier, and in some aspects, a depot support structure, which may or may not comprise suspension or attachment components. Active agent-delivering degradable medical devices and/or compositions may comprise absorbable and/or non-absorbable components and may comprise components to minimize the risk of occluding critical lumens associated with a treated body cavity, for example, the urethra, i.e. to prevent blocking urine flow, and components to maintain the location of an implanted active agent-delivering degradable medical device and/or composition within the body cavity for the desired duration of release of one or more active agents.

In an aspect, a disclosed active agent-delivering degradable medical device and/or composition may comprise one or more components such that at least a portion of the medical device and/or composition will degrade within a subject, wherein degradation of all or a portion of the degradable medical device and/or composition is controlled so as to occur in a desired manner, for example, by physical or chemical actions. As used herein, degradable medical devices and/or compositions or portions of medical devices and/or compositions that degrade after being implanted into a subject may be referred to herein as a bioabsorbable, biostable, bioresorbable, biodegradable, resorbable, naturally dissolving, biodegradable, disintegratable, erodible or bioerodable, soluble or biosoluble. Each of these terms may be used interchangeably with another. Medical devices and/or compositions or portions of medical devices and/or compositions that do not degrade after being implanted are referred to herein as nonbioabsorbable, nonbiostable, nonbioresorbable, nonbiodegradable, nonresorbable, nondegradable, not soluble, not bioerodable, or not naturally dissolving. Each of these terms may be used interchangeably. In an aspect herein, one or more active agents may be provided in a resorbable or nonresorbable portion of a medical device and/or composition, which may comprise the degradable medical device and/or composition structure, components of the medical device and/or composition such as a coating or a containment layer, or two or more of these. For example, one or more active agents may be provided in a resorbable portion of a nonresorbable medical device and/or composition component.

In an aspect, a medical device and/or composition may include a containment layer that surrounds all or a portion of the medical device and/or composition, wherein the containment layer is constructed in such a way that the medical device and/or composition degrades in a manner different from the manner (e.g., time of degradation and/or chemical or physical degradation process) than the medical device and/or composition would degrade absent the containment layer. For example, a medical device and/or composition disclosed herein may purposely include components or physical/chemical characteristics that are particularly vulnerable to degradation. For example, a medical device and/or composition may have sites where degradation will preferentially occur vis-h-vis other sites of the medical device and/or composition. In an aspect, a component or physical/chemical characteristic may be present to impact the degradation profile of the medical device and/or composition. In an aspect, a portion of a medical device and/or composition may be contacted by or exposed to a chemical composition, by radiation, heat, microwave energy, or other methods that cause the contacted/exposed portion of the medical device and/or composition to degrade faster or slower than other portions of the medical device and/or composition. In an aspect, a medical device and/or composition may contain a compositional vector, which means that the composition of the medical device and/or composition will vary along a dimension, e.g., along the length of the medical device and/or composition. The varying composition will have a corresponding varying susceptibility to degradation under the conditions to which the medical device and/or composition is exposed within the subject. For example, one end of a medical device and/or composition can be caused to degrade before the other end degrades. As an example, a medical device and/or composition may include a compositional inhomogeneity where the site(s) of the inhomogeneity are either more or less susceptible to degradation than are the neighboring homogeneous sites of the medical device and/or composition. For example, a medical device and/or composition may include particles (e.g., components) dispersed in a polymer, where the polymer is homogeneous and the particles provides an inhomogeneity that is more susceptible to degradation than is the polymer, or acts as an initiation site for degradation of the polymer. These are nonlimiting examples of controlled degradation according to the present disclosure, whereby medical devices that degrade within a subject are constructed in such a way that degradation is controlled so as to occur in a desired manner due to components, physical or chemical features that are incorporated into a medical device and/or composition.

In an aspect, the present disclosure comprises a degradable medical device and/or composition comprising a containment layer that at least partially encases a medical device and/or composition, the medical device and/or composition being at least partially degradable when the medical device and/or composition is implanted in a subject, the containment layer may be either nonbiodegradable or biodegradable, however when the containment layer is biodegradable, it degrades either more slowly or more quickly than the portion of the medical device and/or composition which it is surrounding.

In an aspect, a containment layer may serve as a container for pieces of the encased medical device and/or composition that form during the degradation of the medical device and/or composition. A containment layer may be a coating on a medical device and/or composition, wherein the coating is optionally hydrophilic. When the coating is biodegradable, it may have a slower or faster rate of degradation than the medical device and/or composition or portion of the medical device and/or composition which is encased by the coating. Alternatively, a coating may degrade at the same or a similar rate as that of the encased medical device and/or composition or the encased portion of the medical device and/or composition.

In an aspect, the present disclosure comprises a medical device comprising a containment layer that is at least partially encased within the medical device comprising a hollow portion, wherein the containment layer at least partially encases a hollow portion (e.g., a cavity formed by walls of at least a portion of the medical device) of the medical device, the medical device being at least partially biodegradable when the medical device is placed within or is implanted within in a subject, the containment layer being either nonbiodegradable or biodegradable. In an aspect, a containment layer provides a barrier between the degradation product(s) of the medical device and/or an associated composition that form during the degradation of the medical device and/or composition and the lumen of the medical device. In an aspect, a containment layer may encase both internal and external surfaces of a medical device by covering all or a portion of the internal and external surfaces of a medical device capable of delivering at least one active agent.

A containment layer may aid in controlling the degradation of the degradable medical device, wherein degradation may include the movement of the degradable device from its initial site of placement in the subject to another anatomical site or to the exterior of the subject's body, e.g., excretion of the degraded device. In an aspect, the containment layer may be a coating that is located on or within a lumen of the medical device and/or on or around a composition provided with the medical device (e.g., herein “associated composition”). In another option, the containment layer may be a mesh that is located on or within the medical device and/or composition. The containment layer may cover only a portion of the medical device and/or associated composition, e.g., the end of a tubular portion of a medical device may have a cap that serves as a containment layer for the lumen of the tubular portion or for containing an associated composition residing in the lumen.

In an aspect, the present disclosure provides degradable medical devices and/or compositions comprising active agents that are located substantially within a cavity in the body, e.g., located within a urinary bladder. As used herein, degradable includes medical devices and/or compositions that are fully degradable or that are partially degradable. As used herein, partially degradable means that a portion of the medical device and/or composition is not degradable or that the medical device and/or composition does not fully degrade during the time frame in which the device and/or composition is located within the body. As used herein, fully degradable, has the meaning that the entire medical device and/or composition is degradable within a desired timeframe. Body cavities of a subject, such as the urinary bladder, abdominal cavity, peritoneal cavity, gall bladder, joint cavities, areas between membranes, such as the pleural cavity, pericardial cavity, and potential spaces, such as the uterus or intermembrane spaces may be treated using methods and devices disclosed herein. For brevity, the urinary bladder is referred to herein, but those of skill in the art can recognize applications of methods, compositions and devices disclosed herein for treatment of body cavities found in subjects.

In an aspect, a degradable medical device and/or composition for treating a subject's body cavity, e.g., a urinary bladder, may be free-floating within the cavity. For example, a free floating medical device and/or composition may comprise a fenestrated solvent-cast film, that may be provided, cut, molded or manufactured into one or more physical shapes, such as having one or multiple layers of the same or different chemical composition, and/or shaped into a two- or three-dimensional form, including but not limited to, a rectangle, a square, a disc, a tube, an annular form, or capsules.

In an aspect, a degradable medical device and/or composition for treating a body cavity, e.g., a urinary bladder, may be attached to a portion of an interior surface of the body cavity. Attachment elements for attaching a disclosed medical device and/or composition may comprise adhesives, one or more barbs or hooks to penetrate the interior surface or wall of the body cavity, loops or structures through which sutures or other known attaching elements, such as staples, transit and secure the medical device and/or composition to an interior surface of the body cavity. For example, an attachable degradable medical device and/or composition may comprise a patch with a microneedle array.

In an aspect, a degradable medical device and/or composition for treating a body cavity, e.g., a urinary bladder, may be embedded at one or more locations within an interior surface or wall of a body cavity. Embedded, as used herein, means that at least a portion of a medical device and/or composition is placed within the wall of a body cavity, i.e., under at least a portion of a surface membrane or layer. For example, in a urinary bladder, all or a portion of a medical device and/or composition may be placed within or between layers of the mucosa, submucosa, muscularis, and serosa or adventitia. A degradable medical device and/or composition, such as a composition comprising one or more active agents, may be embedded. In an aspect, a composition may comprise a gel comprising one or more active agents. In an aspect, a degradable medical device and/or composition may comprise a microparticulate carrier comprising microparticles comprising one or more active agents, or a carrier comprising one or more active agents wherein microparticles may aid in creating “channels” or voids within the carrier which aid in release of one or more active agents.

The present disclosure comprises methods and degradable medical devices and/or compositions for making and using degradable medical devices and/or compositions for delivery of active agents disclosed herein, where any of the disclosed degradable medical devices and/or compositions may be modified to exhibit controlled degradation and/or controlled release of active agents as disclosed herein.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more aspects are set forth in the description below. The features illustrated or described in connection with one exemplary aspect may be combined with the features of other aspects. Thus, any of the various aspects described herein can be combined to provide further aspects of a disclosed medical device and/or composition, or method of making or using disclosed medical devices and/or compositions. Aspects can be modified, if necessary to employ concepts of the various patents, applications and publications as identified herein to provide yet further aspects. Other features, objects and advantages will be apparent from the description, and the claims.

### DETAILED DESCRIPTION

The present disclosure may be understood more readily by reference to the following detailed description and the Examples included herein. It is to be understood that the terminology used herein is for the purpose of describing specific aspects only and is not intended to be limiting. It is further to be understood that unless specifically defined herein, the terminology used herein is to be given its traditional meaning as known in the relevant art. The headings used within this document are only being utilized to expedite its review by the reader, and should not be construed as limiting the disclosure or claims in any manner.

As used throughout this document, including the claims, the singular form “a”, “an”, and “the” include plural references unless indicated otherwise. For example, “a” polymer may include one or more polymer. As another example, “a” layer refers to one or more layers.

“Degrade” as in a degradable medical device and/or composition means that the medical device and/or composition, when implanted into a subject at a location in the subject that is intended for the device, will break down or deteriorate in either a chemical or structural sense. For example, a device that breaks into pieces, e.g., it breaks in half or it disintegrates into many pieces, is a device that has degraded in a structural sense. When the device softens while implanted, that device degrades in a structural sense. When some or all of a device dissolves into the biological fluid with which the device is in contact, then that device chemically degrades. Chemical degradation also includes the occurrence of degradation reactions such as hydrolysis, oxidation, and enzymatic bond cleavage. A degradable medical device and/or composition is a device or composition that will resorb or disintegrate in the subject. A degradable medical device and/or composition refers to a medical device and/or composition that is intended by the manufacturer and/or the health care provider who provides the device to have a desirably limited lifetime in the subject. In other words, the manufacturer and/or health care provider has made and/or selected the device, in part, because it should naturally degrade in the subject and not become a permanent fixture within the subject.

“Degradation profile” refers to a description of how the degradable medical device and/or composition degrades. The degradation profile may provide a time course for the medical device and/or composition degradation as well as a geometric description of the degradation during the time course. For example, a degradable medical device and/or composition may have a degradation profile whereby the medical device and/or composition degrades in some amount, for example, along its length from top to bottom over the course of a specified number of days.

As used herein, “implanted” or “implantable” refers to attaching or providing a device to a subject, such as by placing the device on or within skin, tissue, a body structure, a body cavity, or an organ. The medical device and/or composition provides medical (as opposed to, e.g., purely cosmetic) purposes or benefits, in that it moderates the health of the subject through any one or more of diagnosing, modulating, preventing, treating or curing a medical condition such as a disease or pathology. In an aspect, a disclosed medical device and/or composition may be a parent (either an independent device or a device used in conjunction with an accessory device), or may be an accessory device which does one or more of: supports the performance of a parent medical device and/or composition by enabling or facilitating that parent device to perform according to its intended use; supplements the performance of a parent device by adding a new function or a new way of using the parent device, without changing the intended use of the parent device; or augments the performance of a parent device by enabling the device to perform its intended use more safely or effectively.

In an aspect, a medical device and/or composition is formed, at least in part, from one or more of a thermoplastic or thermoset or elastomeric polymer. In an aspect, a medical device and/or composition is sterile. In an aspect, a disclosed medical device and/or composition is intended to be wholly implanted into the subject, i.e., to entirely be contained by a body cavity and/or a related structure, as opposed to, e.g., a hearing aid that sits in the ear, a dental prosthetic which sits in the mouth of the subject, or a contact lens which sits on the eye of a subject. In one aspect, an implantable medical device and/or composition is intended to be implanted in a body passageway such as a tube or vessel, or to bridge between body structures through a tube or vessel so as to provide at least one active agent to one or both body structures, e.g., intravesical administration, and optionally to the tube or vessel. Implantable medical devices and/or compositions are also described in the following patent documents: U.S. Pat. Nos. 8,753,387; 8,101,104; 7,594,928; and US 2014/0288636, which are herein incorporated for their teachings of medical devices and/or compositions.

In an aspect, an implantable degradable medical device and/or composition comprises a device of the present disclosure comprising a degradable implantable medical device and/or composition and at least one active agent, and optionally, a containment layer may be provided to encompass at least a portion of the medical device and/or composition.

In an aspect, the present disclosure provides implantable medical devices and/or compositions comprising a bioabsorbable medical device and/or composition, at least one active agent, and a containment layer therefor. During the process of bioabsorption within a subject, all or a portion of the medical device and/or composition degrades. In order to manage this degradation process, e.g. to manage the timing of the degradation, the type of degradation, the extent of degradation, and/or the movement of the degraded medical device and/or composition including portions thereof within the subject, the medical device and/or composition may comprise one or more containment layers. A containment layer may, for example, enclose and contain the body of the medical device and/or composition so as to stop the degradation pieces of the device from dispersing within the subject and possibly damaging neighboring tissue and/or organs. In an aspect, the containment layer at least partially, and optionally fully, encases the medical device and/or composition. When the medical device and/or composition degrades into pieces, the containment layer will encase the disintegrating medical device and/or composition and will maintain sufficient structural integrity to hold, or at least assist in holding, the pieces together within its confined space. The containment layer influences and directs the elimination of the medical device and/or composition from the subject, including any pieces that form therefrom.

In an aspect, the present disclosure provides a degradable medical device and/or composition comprising at least one active agent and can provide continuous administration of the one or more active agents. As used herein, degradable means that a device or composition has at least a portion of the device or composition that is degradable in physiological conditions. The portion that is degradable may be less than the entire device and/or composition, or may be the entire device and/or composition. The active agent may act locally in a body cavity or an organ forming the body cavity, or may be absorbed into the blood stream and provide systemic effects to the subject.

In an aspect, the present disclosure provides a nondegradable medical device and/or composition comprising at least one active agent and can provide continuous administration of the one or more active agents. As used herein, nondegradable means that a device or composition has at least a portion of the device or composition that is not degradable in physiological conditions. The portion that is not degradable may be less than the entire device and/or composition, or may be the entire device and/or composition. The active agent may act locally in a body cavity or an organ forming the body cavity, or may be absorbed into the blood stream and provide systemic effects to the subject. If not specifically noted, a medical device and/or composition disclosed herein may be degradable or nondegradable, in whole or in part.

In an aspect, the present disclosure provides an active agent-delivering degradable medical device and/or composition, comprising at least one active agent so that the device provides continuous administration of the one or more active agents while placed within (implanted in) a body cavity. In an aspect, an active agent-delivering degradable medical device and/or composition comprises components for controlled delivery of pharmaceutical agents locally to a body cavity, such as in the bladder. For example, local controlled delivery may comprise whole-organ delivery via release from within the body cavity, or from within the organ wall which forms the cavity, and/or localized delivery to specific areas within the body cavity, e.g. for intratumoral delivery of a chemotherapeutic agent.

The present disclosure may include active agent-delivering degradable medical devices and/or compositions comprising one or more of a pharmaceutically active agent, a depot carrier, and in some aspects, a depot support structure, which may or may not comprise suspension or attachment components. Active agent-delivering degradable medical devices and/or compositions may comprise absorbable and/or non-absorbable components, and may comprise components to minimize the risk of occluding critical lumens associated with a treated body cavity, for example, the urethra, i.e. to prevent blocking urine flow, and components to maintain the location of an implanted active agent-delivering medical device and/or composition within the body cavity for the desired duration of release of one or more active agents. As used herein, a degradable medical device and/or a composition also comprises a drug delivery system, wherein a drug delivery system comprises a matrix that further comprises an active agent that is incorporated into the matrix and wherein the active agent is released from the matrix over a period of time. As used herein, a degradable medical device and/or a composition” may mean that all or a portion of the medical device is degradable, or that all or a portion of a composition is degradable, or that both all or a portion of the medical device and the composition are degradable.

Active agent-delivering degradable medical devices and/or compositions disclosed herein may include, but is not limited to, 1) free floating devices, wherein the device does not comprise attachment components, so that the device may move freely within the body cavity; tacked or affixed devices, wherein the device comprises components for securing the device onto an internal surface of a body cavity, or the device is shaped so that it can be secured on an internal surface of a body cavity, so that the device delivers one or more active agents into the body cavity; and/or 2) embedded devices, wherein at least a portion of the device is implanted within the wall of the cavity such that at least one active agent is delivered into the wall of the body cavity for local and/or systemic delivery of at least one active agent.

Additionally, the present disclosure comprises degradable medical device and/or a composition and methods of treatments wherein a degradable medical device and/or a composition only is provided, and does not comprise one or more active agents, is administered to a subject, and after administration of the degradable medical device and/or a composition, at least one active agent is then provided to the device. In an aspect, a method of administration of at least one active agent to or at a body cavity, comprises administering a disclosed degradable medical device and/or composition to a body cavity, e.g., implanted thereon or therein or provided thereto, wherein the degradable medical device and/or composition does not contain one or more active agents, e.g., the degradable medical device and/or a composition is administered separately from one or more active agents. In a separate subsequent, sequential or concurrent step, after administration of the device, one or more active agents are administered so as to be combined with the degradable medical device and/or a composition so that the one or more active agents are then delivered by the degradable medical device and/or composition. In a method of treatment, one or more active agents may be administered to a degradable medical device and/or a composition one time or multiple times. Multiple times may comprise a series of administrations of one or more active agents, which active agents may be the same or different active agents, or doses or formulations, or may comprise intermittent administrations as needed by the subject. Active agents that have challenging storage and stability characteristics may be delivered in such methods. Further, administrations of one or more active agents may comprise administration of the same or differing amounts of one active agent or the same or differing amounts of more than one active agent. A series of administrations of one or more active agents may comprise administering in the series an increasing amount of one or more active agents or a decreasing amount of one or more active agents, or both an increasing amount of one or more active agents and a decreasing amount of different active agent or agents. In an aspect, in a first administration, one or more active agent(s) is administered to a degradable medical device and/or a composition, and in a subsequent administration, a second, third or more active agent(s) is administered or may be co-administered with the first active agent(s). In an aspect, in a first administration, a particular amount of one or more active agent(s) is administered to a degradable medical device and/or a composition, and in one or more subsequent administration(s), a larger or smaller amount of the same active agent is administered. One or more active agents may be administered in this manner of altered amounts. Such administrations may be repeated as needed by the subject. One of skill in the art can determine amounts of active agents to be delivered to an implanted device disclosed herein so that an effective amount of one or more active agents is delivered to the subject in need.

Generally as used herein, an active agent is mixed, admixed, dissolved or suspended in a pharmaceutically acceptable composition. Such a composition may comprise compounds or molecules such as diluents, excipients, solution- or diffusion-enhancing compositions, surfactants, buffers, porogens, pH modulators, anti-oxidants, lipids, salts, vitamins, energy molecules (e.g., ATP, glucose) or other known formulation compounds or molecules for treatments or diagnoses of subjects.

A series of disclosed medical devices and/or compositions may be used, such as provided in a kit, so that each device comprises a composition comprising an amount of at least one active agent. A method of administering at least one active agent to a subject comprises serially administering to the subject two or more degradable medical devices and/or a compositions comprising at least one active agent, such as those from a kit, so that the first administered degradable medical device and/or a composition delivers at least a portion of its active agent, and after a desired time period, a second degradable medical device and/or a composition is administered, (e.g., implanted) and delivers a portion of its active agent, and so on for the rest of the devices and/or compositions in the series, such as those of the kit. The first, or a subsequent device and/or composition may or may not be removed from the subject. The first, or a subsequent degradable medical device and/or a composition may or may not comprise the same active agent, or may or may not comprise the same dose of one active agent or the same dose of different active agents. A degradable medical device and/or a composition of a series of devices and/or compositions may or may not comprise the same active agent, or more than one active agent or differing combinations of active agents. The amount of at least one active agent may differ from degradable medical device and/or a composition to degradable medical device and/or a composition in the series by increasing or decreasing amounts, or by the type, number or amount of active agents provided by a degradable medical device and/or a composition.

A method of administering at least one active agent to a subject comprises simultaneously (in parallel) administering to the subject two or more degradable medical device and/or a composition, such as those from a kit, so that the each administered degradable medical device and/or a composition delivers at least a portion of its active agent, and a second degradable medical device and/or a composition is concurrently administered (e.g., implanted or provided) and delivers a portion of its active agent, and so on for the rest of the degradable medical devices and/or compositions in the plurality of degradable medical devices and/or compositions administered, such as those of the kit. The first, or subsequent degradable medical devices and/or compositions may or may not be removed from the subject. The first, or a subsequent degradable medical device and/or a composition may or may not comprise the same active agent, or may or may not comprise the same dose of one active agent or the same dose of different active agents. A degradable medical device and/or a composition of a plurality of degradable medical devices and/or compositions administered concurrently may or may not comprise the same active agent, or more than one active agent or differing combinations of active agents. The amount of at least one active agent may differ from degradable medical device and/or a composition to degradable medical device and/or a composition in the concurrent plurality of degradable medical devices and/or compositions by increasing or decreasing amounts, or by the type, number or amount of active agents provided by a device and/or composition.

A method of administering at least one active agent to a subject comprises administering to the subject at least one degradable medical device and/or a composition, so that the administered degradable medical device and/or a composition delivers at least a portion of its active agent. One or more degradable medical device(s) and/or composition(s) may be provided in a kit. A degradable medical device and/or a composition may or may not be removed from the subject.

In an example for illustration and not for limitation of the disclosure, a body cavity of a subject is the urinary bladder. Currently, it is difficult to locally deliver an active agent to the bladder other than by flushing the bladder with a solution. The bladder is a body cavity defined by a dynamic flexible wall which allows the bladder to expand to almost two times its smallest volume. The interior cavity surface is a specific, dense transitional epithelium layer which, along with a mucous layer, forms a barrier to minimize urine reabsorption back into the body. Fluids within the bladder cycle through multiple times in a day and vary in pH widely.

The present disclosure comprises treatments for bladder pathologies and diseases. For example, and not for limitation, bladder cancer treatment is discussed. Those of skill in the art can adapt such discussion for other bladder pathologies, which are included herein. Bladder cancers pose a particular challenge for treatment and no significant therapeutic advances have been made recently. Common bladder cancers are found in the mucosal layers and are generally treated with antibiotic chemotherapy by using a Foley catheter to retain mitomycin-c within the bladder for short-term, e.g., one hour, treatments on a weekly basis. Recurrence is common, and occurs throughout the bladder, not just at the original lesion site. More invasive tumors, found within the muscle layers, have a low survival rate. Stage IV cancers involving the muscle tissue have a 15% survival rate at 5 years. About 60,000 cases are diagnosed in the US annually. Methods, devices and compositions disclosed herein are effective for treatment of bladder disease, particularly cancers of the bladder, regardless of location. A method of treatment of bladder cancer comprises administering at least one of a disclosed degradable medical device and/or a composition comprising an effective amount of at least one active agent to the bladder of a subject having bladder cancer, or a subject diagnosed with bladder cancer, or a subject previously treated for bladder cancer. A method of treatment of bladder infection or chronic bladder infection, comprises, administering a disclosed degradable medical device and/or a composition comprising an effective amount at least one active agent to the bladder of a subject having bladder infection or chronic bladder infection, or a subject diagnosed with bladder infection or chronic bladder infection, or a subject previously treated for bladder infection or chronic bladder infection.

In an aspect, a degradable medical device and/or a composition disclosed herein comprises at least an active agent-eluting core composition that is encapsulated by an encapsulating member comprising one or more of all or a portion of a medical device, a containment member, or a coating. In an aspect, a degradable medical device and/or a composition comprises at least an active agent composition that may or not be encapsulated.

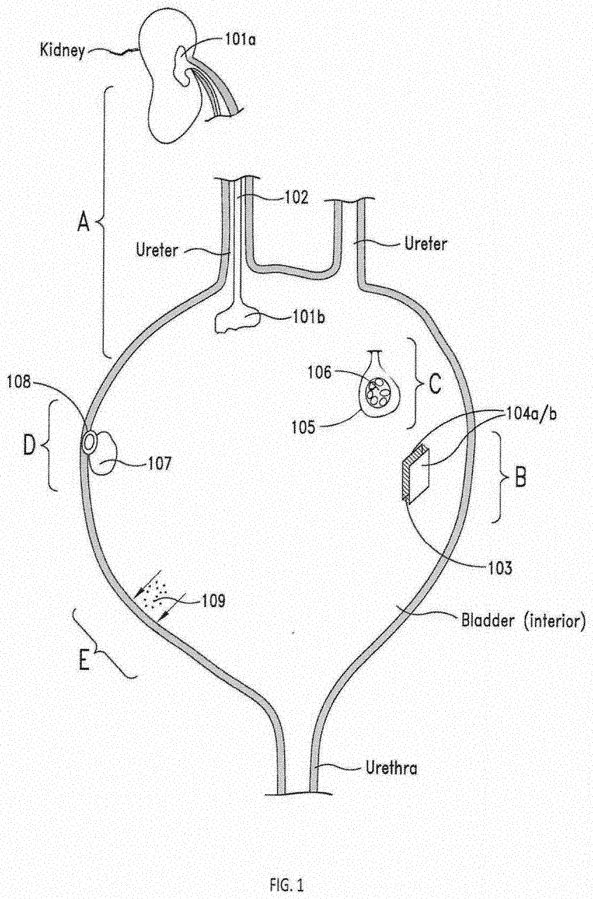

In an aspect, a disclosed degradable medical device and/or a composition comprising an active agent-eluting core that is encapsulated is a stationary degradable medical device and/or a composition and comprises at least one retaining element positioned in another organ or in a lumen or both. For example, an exemplary stationary degradable medical device and/or composition is shown in FIG. 1 as Device A, comprising a tubular section, comprising a wall that defines an interior hollow cavity, and at least one retaining element, wherein two retaining ends, 101a and 101b, are shown in relation to placement of Device A within a urinary bladder. One or both of retaining elements 101a and/or 101b, may be present in device A. As shown the urinary bladder comprises two ureters, which fluidly connect two kidneys (only one is shown, not to scale) to the urinary bladder. The urethra provides the exit for urine from the urinary bladder. Retaining ends 101a/b may be shaped so that retaining end 101a/b remains within the organ in which it is originally placed, e.g., a kidney or the urinary bladder, and functions to maintain the placement of tubular portion 102. Shapes include, but are not limited to, coils, rings, umbrella shapes, T shapes, and hooks. Tubular portion 102 resides within at least a portion of a ureter, and may traverse the entire ureter from kidney to bladder. One or more active agents may be delivered from one or more of retaining end 101a/b, from tubular portion 102, from a composition positioned within the interior hollow cavity of tubular portion 102, from a coating provided on one or more of retaining end 101a/b, and/or from tubular portion 102. Such degradable devices are disclosed herein. For example, an active agent-eluting core composition may comprise a composition positioned within the interior hollow cavity of tubular portion 102 which comprises at least one active agent, and the interior hollow cavity of tubular portion 102 encapsulates the active-agent eluting core composition. In an aspect, an active-agent eluting composition may comprise a coating on Device A wherein the coating comprises at least one active agent.

In an aspect, a disclosed degradable medical device and/or a composition comprises a delivery device that is free-floating, e.g., not physically attached to any body structure and is able to move freely, within a body cavity. Device A is not a free-floating delivery device, in that Device A is not able to move freely, but is constrained by its shape or tethered so that it maintains its position, at least within a ureter, until resorption of at least a biodegradable portion of Device A so that Device A is no longer tethered in place. A free-floating device remains within a body cavity, e.g., a urinary bladder, but is not physically attached to the body cavity or any associated organs, or is not tethered by a portion of the device located in an adjacent or related organ or lumen.

In an aspect, a free-floating active agent delivery degradable medical device and/or a composition comprises a film and at least one active agent. One or more active agent(s) can be incorporated into a film during the manufacture of the film, or it can be incorporated into a film after the film has been manufactured. A film can be manufactured by solvent casting, extrusion, additive manufacturing or injection molding. At least one active agent can be blended with one or more of the materials used to make films and then a film containing at least one active agent can be manufactured. For the solvent casting process, at least one active agent can be soluble in the solvent used to dissolve film materials. In an aspect, at least one active agent can be partially soluble in the solvent used to dissolve film materials such that both soluble and particulate active agent are present in the final film. In an aspect, at least one active agent can be virtually insoluble in the solvent used to dissolve film materials such that the active agent is present as a particulate in the final film. At least one active agent can be incorporated into the body of the film (film construct) after the film has been manufactured. In an aspect, at least one active agent can be dissolved in a solvent and then the film construct can be immersed into the solution comprising the at least one active agent. The solvent used to dissolve the at least one active agent does not dissolve the film. In an aspect, the solvent can swell the film. After a period of time, the film construct can be removed from the solution and can be dried. In an aspect, the film construct can be rinsed with a solvent to remove any surface-associated active agent. In an aspect, an at least one active agent solution can be sprayed onto a film construct. An at least one active agent-containing film can be laminated to a second film. In an aspect, the second film can comprise at least one active agent. In an aspect, the at least one active agent can be the same agent as in the first film. In an aspect, the at least one active agent can be a different agent than that in the first film. In an aspect, the second film could comprise two or more active agents. In an aspect, the second film can comprise polymer compositions that are different than that of the first film. In an aspect, the second active agent-containing film can release at least one active agent at a release rate that is different to that at which the first film releases an active agent. In an aspect, the second film can contain no active agent. In an aspect, the diffusion of the at least one active agent through the second film can be slower than that of the at least one active agent through the first film such that the release of the active agent from the laminate is greater in one direction. In an aspect, the second film can be a solid film. In an aspect, the second film can comprise a non-continuous film. The second film can comprise fenestrations, pores, holes, gaps or a combination thereof. The delivery system can comprise a trilaminate format in which the system comprises three distinct layers. In an aspect, the middle layer can comprise one or more active agents and the outer two layers can comprise no active agent. The two outer layers can comprise polymer compositions that are different from each other such that the release rate of the active agent is more rapid through one side of the laminate than the other side. The three distinct layers of the trilaminate structure can each comprise one or more active agents. In an aspect, the active agent can be the same. In an aspect, the at least one active agent can be a different active agent in each layer. In an aspect, two layers can comprise the same at least one active agent which is different from the at least one active agent in the third layer. The trilaminate layer can have one or more active agents in two of the three layers. In an aspect, the active agents are the same. In an aspect, the active agents are different. Based on a similar approach to the bilaminate and trilaminate systems, systems with four, five or six layers can be used.

In an aspect, both eluting and non-eluting layers used in the preparation of films may be formed by polymers disclosed herein. In an aspect, a film construct, e.g. Device B, whether single- or multi-layer, may be made of polymeric compositions that are initially stiff and resistant to bending, and as at least a portion of the polymers degrade, the construct become less resistant to bending and more pliable with time. In an aspect, a film construct, e.g. Device B, whether single- or multi-layer, may be made of polymeric compositions that are initially stiff and resistant to bending, and as at least a portion of the polymers become hydrated, the construct become less resistant to bending and more pliable. In an aspect, a film construct, e.g. Device B, whether single- or multi-layer, may further comprise a retrieval element. For example, a retrieval element may comprise a string or flexible member that can be grasped so that the film construct is removed from the body cavity through an exit canal, such as removing Device B (retrieval element not shown) from the urinary bladder through the urethra. Such removal could occur at any point during the at least one active agent-eluting treatment, such as when early removal prior to completion of a desired amount of at least one active agent, or after an effective amount of at least one active agent has been delivered. A single-, or multi-layered, construct may be shaped in any known manner, including but not limited to, a sheet, disc and/or formed into a three dimensional tube. In an aspect, the film can have the shape that includes but is not limited to a square, a rectangle, a rhombus, a circle, an annulus, a torus or a triangle.

In an aspect, degradable medical device and/or a composition can comprise a polymeric matrix that is fully or partially encased in a flexible polymeric substrate. In an aspect, a degradable medical device and/or a composition can comprise a polymeric matrix that is fully or partially attached to a polymeric substrate. The polymeric substrate can include but is not limited to a film, a foam, a mesh or a combination thereof. The polymeric matrix can comprise one or more active agents. The polymeric matrix can further comprise an excipient. The polymeric matrix can be in the form of a rod, a bar, a disc or a combination thereof. In an aspect, a bar can comprise a rectangular shape, a square shape, a rhomboid shape, a trapezoid shape or a combination thereof. The polymeric matrix can have a length of about 10 mm to about 100 mm. The polymeric matrix can have a width of about 0.5 mm to about 10 mm. In an aspect, the polymeric matrix can have a width of about 0.5 mm to about 5 mm. In an aspect, the polymeric matrix can have a width of about 0.5 mm to about 2 mm. In an aspect, a polymer matrix can be sliced into sections. The sections can have a thickness of about 0.5 mm to about 10 mm. In an aspect, the sections can have a thickness of about 0.5 mm to about 5 mm. In another aspect, the sections can have a thickness of about 1 mm to about 3 mm.

A polymeric matrix or sections thereof can be attached to a flexible polymeric substrate. A polymeric matrix or sections thereof can be attached to the substrate using and adhesive composition. In an aspect, an adhesive composition can comprise a polymer as described herein and a solvent. The adhesive composition can be applied to the polymer matrix, the polymeric substrate or a combination thereof. Once the adhesive has been applied and the polymer matrix is adhered to the polymeric substrate, the solvent is allowed to evaporate and the polymer matrix is left adhered to the polymeric substrate. In another aspect, the polymer matrix can be attached to the polymeric substrate by a solvent welding process. A solvent for either the polymer matrix or polymeric substrate is applied to the surface of the matrix or substrate to be attached. The matrix and substrate are brought together and a force is applied. The solvent is allowed to evaporate. The matrix and substrate can be adhered together using a hot melt process or a hot melt adhesive. In an aspect, the matrix and substrate can be adhered together using a self-curing adhesive. In an aspect the self-curing adhesive can comprise a cyanoacrylate adhesive composition.

The polymer matrix or a section thereof can be placed on a flexible polymeric substrate. This process can be repeated until a series of polymer matrices or sections cover the polymeric substrate. A second polymeric substrate can then be applied over the top such that the polymer matrix or section thereof is sandwiched between the polymeric substrates. In an aspect, the top and bottom polymeric substrate can comprise the same composition. In an aspect, the top and bottom polymeric substrate can comprise a different composition. In an aspect, the top and bottom substrate layers can be adhered together using an adhesive, a solvent welding process, a hot melt process, a hot melt adhesive, a cyanoacrylate adhesive or a combination thereof. In another aspect the substrates can be selected such they adhere to each other without the use of an adhesive, solvent or heating process.

In an aspect, adegradable medical device and/or a composition in the form of a film or laminate could be rolled into a tubular shape such that the degradable medical device and/or a composition could be loaded into a catheter or the working channel of a cystoscope. The catheter or cystoscope can be used to enter the body cavity after which the film or laminate system is ejected from the catheter or cystoscope. In an aspect, a free-floating active agent delivery degradable medical device and/or a composition comprises a film construct, comprising a solid or gel-based core layer comprising at least one active agent, and the active agent-eluting film may or may not be bonded to at least one layer, or to two layers positioned so that one layer is positioned on each surface of the film, and which may or may not be active-agent eluting layers, wherein the film comprises one or more multiple layers of solid or semi-solid materials for controlled release of at least one active agent. A single-, or multi-layered, film construct may be shaped in any known manner, including but not limited to, a sheet, disc and/or formed into a three dimensional tube. See FIG. 1, Device B, wherein active agent-eluting film 103 is shown sandwiched between two non-eluting layers 104a/b. Non-eluting layers may have opening therethrough for fluid flow across the construct. Non-eluting layers 104a/b may be formed by melt processing or solvent processing techniques and could be formed in one step (e.g., by multi-layer extrusion) or applied progressively (e.g. by lamination).

In an aspect, both eluting and non-eluting layers may be formed by polymers disclosed herein. In an aspect, a film construct, e.g. Device B, whether single- or multi-layer, may be made of polymeric compositions that are initially stiff and resistant to bending, and as at least a portion of the polymers degrade, the construct become less resistant to bending and more pliable with time.

In an aspect, a film construct, e.g. Device B, whether single- or multi-layer, may further comprise a retrieval element. For example, a retrieval element may comprise a string or flexible member that can be grasped so that the film construct is removed from the body cavity through an exit canal, such as removing Device B (retrieval element not shown) from the urinary bladder through the urethra. Such removal could occur at any point during the drug-eluting treatment, such as when early removal prior to completion of a desired amount of at least one active agent, or after an effective amount of at least one active agent has been delivered.

In an aspect, a free-floating active agent delivery degradable medical device and/or a composition comprises an indwelling catheter, which has a delivery end positioned within a body cavity, and an attachment end that is attached to a container of a composition comprising an at least one active that is acted on by an infusion pump. A catheter delivery end may comprise end structures, such as a cup or balloon, for maintaining the catheter delivery end within the body cavity. For example, a container may be a syringe with a plunger that is moved by an infusion pump, or may be an infusion container for which an infusion pump controls the release of the contained composition.

In an aspect, a free-floating active agent delivery degradable medical device and/or a composition comprises an indwelling stent, which has a delivery end positioned within a body cavity, and an attachment end that is attached to an osmotic pump for delivery of at least one active agent. For example, an osmotic pump device comprises a drug core (reservoir), an osmotic agent, and a semipermeable membrane (rate controller). In addition, a flow moderator may be inserted into the body of the osmotic pump after filling. This is a type of implantable or insertable system in which the active agent is in a solution or suspension contained in a cylindrical reservoir formed from a synthetic, collapsible, impermeable elastomer wall (e.g., polyester) that is open to the external environment via a single orifice. In-dwelling stents known to those of skill in the art are contemplated by the present disclosure.

In an aspect, a free-floating active agent delivery medical device and/or composition comprises a three-dimensionally printed (additive manufactured) capsule comprising a core comprising at least one active agent, and a coating, which may or may not comprise at least one active agent. The coating and/or the core may have a plurality of openings which allow for adding a composition comprising at least one active agent, and/or allow for elution from the core and/or coating of at least one active agent. The shape of the capsule, the porosity of the core and/or the coating, the thickness of the core and/or the coating, and the polymeric materials from which the core and/or the coating are made may modulate release of at least one active agent from the capsule. In an aspect, when positioned within a body cavity, a capsule may be shaped so that openings into or out of the cavity are not blocked. For example, in a urinary bladder, a capsule may be shaped so that the ureters and the urethra are not blocked by the capsule.

In an aspect, a free-floating active agent delivery degradable medical device and/or composition comprises a high surface area nonwoven polymeric matrix comprising at least one active agent. Such a polymeric nonwoven matrix may be formed by electrospinning, melt-blending, and/or other known methods, polymeric compositions comprising at least one active agent into fibers that are assembled to form a nonwoven polymeric matrix. In an aspect, a free-floating active agent delivery degradable medical device and/or a composition comprises a high surface area nonwoven polymeric matrix onto which at least one active agent is applied by electrowriting a composition comprising an active agent. Nonwoven polymeric matrices may be shaped in a desired form, for example, formed into a tube or other three-dimensional construct. A nonwoven polymeric matrix comprising an active agent may be coated with a polymeric composition that aids in control of at least one active agent. Such a coating may be continuous or may be interrupted, e.g., a coating applied in stripes on the matrix.

In an aspect, a free-floating active agent delivery degradable medical device and/or a composition comprises an encapsulated delivery bladder. See FIG. 1, Device C, wherein the active agent delivery bladder device comprises closed container 105, which encapsulates and contains composition 106 comprising at least one active agent. Such an active agent delivery bladder device may be provided to a body cavity by a trocar or catheter. In an aspect, an active agent delivery bladder device may further comprise a coating, comprising PEG or a high modulus polymeric composition, which is lubricious and allows the active agent delivery bladder device to be delivered easily through a catheter or trocar. The delivery bladder device may be made from polymeric materials, such as thermoplastic polyurethane (TPU), silicones or degradable low crystalline, flexible polyaxial block copolymers comprising ester linkages, e.g., SVG12 (a poly-axial lactide/ε-caprolactone polymer). In an aspect, the active agent delivery bladder device comprises microchannels within its wall to enable active agent transfer across the wall, for example, by hydrophilic interactions, or for modulation of surface energy and/or wettability of the bladder wall interior or exterior surfaces. In an aspect, the active agent delivery bladder device comprises one or more openings therethrough the delivery bladder device wall to allow fluid transport into and/or out of the bladder For example, an active agent delivery bladder device with openings could be made by solvent casting a balloon or bladder from a polymeric composition comprising low crystalline, flexible polyaxial block copolymers comprising ester linkages with a minor amount of polyethylene glycol (PEG). A composition comprising at least one active agent is then instilled within the active agent delivery bladder device. Once in position in a body cavity, the PEG dissolves, leaving openings or microchannels within the bladder/balloon wall, which function in transferring or eluting one or more active agents in the composition contained by the balloon.

In an aspect, an active agent delivery bladder device may comprise a gas reservoir contained within the bladder. Such a gas reservoir aids in the delivery bladder floating within the fluids of a body cavity, and may aid in orienting the delivery bladder within such fluids. In an aspect, an active agent delivery bladder device may comprise a composition comprising at least one active agent, a composition comprising at least one active agent in a pharmaceutically effective solution or suspension, a composition comprising at least one active agent in a release matrix, a composition comprising at least one active agent in a pharmaceutically effective solution or suspension that aids in stability of at least one active agent and/or release of at least one active agent in a controlled manner to provide a treatment time, or combinations thereof.

In an aspect, a free-floating active agent delivery medical device and/or composition comprises a an active agent delivery bladder device which comprises a flexible container (e.g., the bladder) containing a composition comprising at least one active agent wherein the flexible container is shaped so that the flexible container is planar, and then is rolled to form a tubular construct having layers of container containing the composition adjacent to one another. The composition comprising at least one active agent can be any composition disclosed herein. In an aspect, a bladder device may be shaped as a pouch that is closed by a drawstring or is pinch-sealed closed.

In an aspect, a free-floating active agent delivery medical device and/or composition comprises an annular ring or a coil which is hollow. Such a hollow annular ring or coil may be filled with a composition comprising at least one active agent. In an aspect, an annular ring or coil may be made from polymeric material disclosed herein. In an aspect, an annular ring or coil may have a coating on a portion of its exterior surface, which may or may not comprise at least one active agent. In an aspect, an annular ring or coil may have opening therethrough its walls for controlled delivery of at least one contained active agent. In a method, an annular ring or coil is placed within a body cavity, and at least one active agent is delivered.

In an aspect, an active agent delivery medical device and/or composition comprises an active agent container, such as devices disclosed herein, that is immobilized by a fastener attaching the active agent container to a surface or muscular layer of a body cavity. See FIG. 1, Device D, wherein active agent container 107, and fastener 108 are shown. For example, a fastener for a degradable medical device and/or composition may comprise one or more sutures, staples, barbs, quills or tacks to immobilize an active agent delivery device to a surface or layer of a body cavity. A degradable medical device and/or composition may have an appendage or an area through which such fastener(s) are used to attach the degradable medical device and/or composition to the body cavity. In an aspect, an active agent degradable delivery medical device and/or composition comprises an active agent container that is a patch, and the patch comprises a microneedle array. Adhesives may be used as a fastener to attach a disclosed degradable medical device and/or composition to a body cavity. An example adhesive comprises a polyacrylamide nanogel. In an aspect, an immobilized degradable medical device and/or composition comprises a central rod having one or a plurality of barbs, such that the degradable medical device and/or composition attaches to a wall of a body cavity by at least one or a portion of its barbs. At least a portion of the barbed device may be coated with a film comprising at least one active agent. In an aspect, the rod may be hollow and is then at least partially filled with a composition comprising at least one active agent. Attachment may be to a surface of a body cavity, and/or to one or more layers forming the wall of the cavity.

In an aspect, free-floating devices may be attached to a wall of a body cavity, and may further comprise an appendage or an area through which fastener(s) are used to attach the degradable medical device and/or composition to the body cavity. For example, active agent-eluting films (i.e., degradable medical device and/or composition) could be shaped as tubes, discs, or other known shapes, and may or may not be coated. Such a film degradable medical device and/or composition may be attached to a body cavity wall by known and disclosed fasteners. In an aspect, a film degradable medical device and/or composition may only be active agent-eluting from one surface so as to provide directed delivery of at least one active agent. For example, a non-eluting surface may be coated to prevent elution of at least one active agent.

In an aspect, an active agent delivery degradable medical device and/or composition is embedded in a surface or muscular layer of a body cavity, for example see Device E of FIG. 1. An active agent delivery medical device and/or composition disclosed herein may be embedded within a wall of a body cavity. A degradable medical device and/or composition such as a gel or in situ polymerizing composition (109 of FIG. 1) comprising at least one active agent may be embedded in a body cavity structure, such as a wall, which includes a surface, layer, fold or other structural element of a body cavity, so that at least one active agent is released in at least one of the body cavity, the circulatory, lymphatic or nervous accessories to the body cavity, and/or systemic systems of the body. For example, a degradable medical device and/or composition comprising a gel carrier such as for example, a viscous poly(ester-ether-ester) interlinked with a diisocyanate or poloxymer, and at least one active agent may be injected into a wall of a body cavity. A degradable medical device and/or composition such as a microparticulate composition comprising at least one active agent may be embedded in a body cavity structure, such as a wall. For example, see FIG. 1, Device E. A degradable medical device and/or composition comprising a plurality of microparticles (109 in FIG. 1) comprising at least one active agent may be embedded in a body cavity wall.

The plurality of microparticles may all comprise one active agent, or may comprise more than one active agent by having each microparticle comprise more than one active agent, or having multiple types of microparticles, each of which comprises a different active agent. High modulus microparticles may comprise channels for penetration of one or more active agents, and may comprise glass, ceramics, and/or absorbable glass. The degradable medical device and/or composition may be rubbed on the body cavity wall or applied by a roller. Minimally invasive procedures can be used to embed disclosed compositions.

In general, degradable medical devices and/or compositions, coatings and containment layers may be made from biostable or non-biostable materials, where biostable materials are referred to herein as degradable materials, and may be known in the art as any of biodegradable, absorbable, bioabsorbable, bioresorbable, biodegradable, resorbable, naturally dissolving, erodible or bioerodable, soluble or biosoluble. Degradable polymers can be completely eroded or absorbed when exposed to bodily fluids, including but not limited to, blood, serum, urine, saliva, and mucous membrane secretions, and can be resorbed, absorbed and/or eliminated by the body. Some degradable materials absorb due to chemical degradation that occurs to the material upon exposure to a bodily fluid such as may be found in the environment of a subject. Chemical degradation refers to degradation of a material due to chemical reaction of the material with bodily fluids or substances within bodily fluids. The chemical degradation can be the result of hydrolysis, oxidation, enzymolysis, and/or metabolic processes, etc. The chemical degradation can result in, for example, a decrease in molecular weight, deterioration of mechanical properties, and decrease in mass due to erosion. Mechanical properties may correspond to strength and modulus of the material. Deterioration of the mechanical properties of the material decreases the ability of a medical device and/or composition made therefrom to function optimally in the subject. For example, if the device is an intravesical device, the device provides diminishing mechanical support or physical presence in the vesical as it degrades. Additionally, some degradable materials are water or saline soluble. A water or saline soluble material refers to a material that is capable of dissolving in water or saline in addition to, or even in the absence of chemical degradation of the material. As referred to herein, saline includes physiologically acceptable saline or for example, interstitial fluid. Degradable materials may disintegrate so as to lose physical or mechanical integrity, for example, to break into smaller pieces. Such smaller pieces can be removed through physiological excretion, for example, urination.

In an aspect, a degradable medical device and/or composition or containment layer is formed, in whole or in part, from a degradable organic polymer. An organic polymer may be, for example, thermoplastic or thermoset or elastomeric polymer. An organic polymer may be a copolymer, where the copolymer is made from two or more different monomers so as to provide properties that are not readily available from a homopolymer. An organic polymer may be in admixture with one or more different polymers, such as one or more different organic polymers. Thus, various degradable organic monomers as identified herein may be used in concert to prepare a homopolymer or a copolymer, and the various organic polymers as identified herein may be used in combination to prepare an admixture. In an aspect, degradable medical devices and/or compositions of the present disclosure are degradable, and accordingly will contain some degradable components. In an aspect, a degradable medical device and/or composition medical device and/or composition is made entirely from degradable materials, and thus the medical device and/or composition is completely degradable. In another aspect, a degradable medical device and/or composition is mostly made from degradable materials, and thus at least 50 wt % of the medical device and/or composition is degradable. In another aspect, a degradable medical device and/or composition is made from both degradable and biostable materials, and thus less than 100% of the medical device and/or composition will degrade. In various aspects, 100% or up to 95%, or up to 90%, or up to 85%, or up to 80%, or up to 75%, or up to 70%, or up to 65%, or up to 60%, or up to 55%, or up to 50%, or up to 45%, or up to 40%, or up to 35%, or up to 30%, or up to 25% of the medical device and/or composition is made from degradable material(s), these percentage values being wt % based on the weight of the degradable medical device and/or composition, and not inclusive of other components such as active agents, coatings, or a containment layer.

Examples of degradable polymers which may be used to prepare a containment layer or medical device and/or composition of the present disclosure include poly(alpha-hydroxy acid) polymers and copolymers, such as polymers and copolymers of glycolide including polyglycolide (PGA), poly(glycolide-co-lactide)(PGLA), and poly (glycolide-co-trimethylene carbonate (PGA/TMC; polymers and copolymers of polylactide (PLA) including poly-L-lactide (PLLA), poly-D-lactide (PDLA), poly-DL-lactide (PDLLA), poly(lactide-co-tetramethylene glycolide), poly(lactide-co-trimethylene carbonate), poly(lactide-co-delta-valerolactone), poly(lactide-co-epsilon-caprolactone), poly(glycine-co-DL-lactide) and poly(lactide-co-ethylene oxide); polydioxanone polymers such as asymmetrically 3,6-substituted poly-1,4-dioxane-2,5-diones; poly(beta-hydroxybutyrate) (PHBA) and copolymers of the same such as poly(beta-hydroxybutyrate-co-beta-hydroxyvalerate); polygluconate; poly(beta-hydroxypropionate) (PHPA); poly(beta-dioxanone)(PDS); poly(delta-valerolactone); poly(ε-caprolactone); methylmethacrylate-N-vinylpyrrolidone copolymers; polyester amides; polyesters of oxalic acid; polydihydropyranes; poly(alkyl-2-cyanoacrylate); polyvinyl alcohol (PVA); polypeptides; poly(beta-maleic acid)(PMLA); poly(beta-alkanoic acid); poly(ethylene oxide) (PEO); polyanhydrides, polyphosphoester, and chitin polymers.

In an aspect, an organic polymer is a polyester. For example, the polymer may be a polyester selected from poly(α-hydroxy acid) homopolymers, poly(alpha-hydroxy acid) copolymers and blends thereof. In addition, or alternatively, the polyester may be selected from polyglycolide, poly-L-lactide, poly-D-lactide, poly-DL-lactide, and blends thereof. The polyester may be selected from polymers and copolymers of polylactide (PLA), including poly-L-lactide (PLLA), poly-D-lactide (PDLA), poly-DL-lactide (PDLLA). Such degradable polyesters are known in the art and are included herein.

In one aspect, the organic polymer is semicrystalline, or is capable of being formed into fibers, or is both semicrystalline and fiber-forming. In one aspect, a containment layer is prepared using an organic polymer that is at least one of semicrystalline and fiber-forming. In one aspect, a degradable intravesical medical device and/or composition is prepared with an organic polymer that is both semicrystalline and fiber forming. To additionally make the organic polymer fast-degrading, glycolide may be used as the, or one of the, monomer(s) used to form the organic polymer. Paradioxane (PDO) is another suitable monomer for forming fast-degrading organic polymers, where the corresponding homopolymer is known as poly(PDO). Poly(PDO) typically degrades more slowly that glycolide-based polymer, so in order to prepare a very fast degrading organic polymer, the monomer feed is preferably rich in glycolide.

In an aspect, an organic polymer has a polyaxial structure, while in another aspect, an organic polymer is linear. The polyaxial structure may be a part of the organic polymer, for example, it may be present in a block of a block copolymer. Another option is for the organic polymer to be a segmented polyaxial that is semicrystalline and fiber-forming, and glycolide-based to ensure fast degradation. Yet another option is to use linear copolymers for either or both of: diblock, triblock, pentablock, wherein the central block is amorphous and the other blocks are semicrystalline, except for the pentablock, which may comprise PEG as the central block with amorphous segments connected to the outer crystalline segments (forming a symmetrical pentablock polymer that is a polyether-ester; all other polymers being referred to are aliphatic polyesters). The linear block copolymers may also be comprised of semicrystalline blocks in all cases, with no amorphous blocks, resulting in polymers that can be oriented after fiber formation to create alternating patterns of different crystalline structure and percentage in the fiber, such that there is slight differences in degradation profile of the alternating blocks forming the fiber (as a fiber is oriented, horizontal strips of crystalline regions form and align the blocks comprising the polymer chain). Alternatively, unblocked linear copolymers can be substituted. In one aspect, these organic polymers are used to form fibers, and the fibers are used to form the containment layer. In another aspect, these organic polymers are not formed into fibers, however the organic polymer is used to form a containment layer, e.g., by simply spraying a solution of the polymer onto the medical device and/or composition, or by dip coating, etc.

Catalysts that can be used to manufacture polyester polymers include but are not limited to a tin-based catalyst, aluminμm-based catalysts, zinc based catalyst and a bismuth based catalyst. Tin-based catalysts that can be used include but are not limited to tin (II) 2-ethylhexanoate. Aluminμm based catalysts that can be used include but are not limited to alμminum isopropoxide, and triethyl aluminum, Zinc based catalysts that can be used include but are not limited to zinc lactate and bismuth based catalysts that can be used include but are not limited to bismuth subsalicylate

In another aspect, the polymers can be random copolymers or block copolymers. Random copolymers can be manufactured by adding 2 or more different monomers to the reaction mixture and allowing the mixture to polymerize. Block copolymers can be manufactured by first adding one or more monomers and allowing the monomers to polymerize and then adding a second monomer that is different from at least one of the first monomers, to the initial polymer and then allowing that to polymerize further. The resultant polymer will thus have a block of similar units linked to a block of similar units that are different from the first units.

In one aspect, the polymer can comprise 50% (w/w) or greater lactide residues. In another aspect the polymer can comprise 60% (w/w) or greater lactide residues. In another aspect, the polymer can comprise 70% (w/w) or greater lactide residues. In another aspect, the polymer can comprise 80% (w/w) or greater lactide residues.

In another aspect, the lactide polymers described can further comprise trimethylene carbonate residues. In another aspect, the lactide polymer can comprise a block of trimethylene carbonate residues and a block of lactide residues. In one aspect, the polymer can be manufactured with an added lactide to trimethylene carbonate ratio of 88:12 (molar ratio).

In one aspect, the initiator used for the polymerization is a hydroxyl-based initiator. In one aspect, the initiator is a diol. In another aspect, the initiator is 1,3 propanediol. In one aspect, the 1,3 propanediol is used to initiate polymerization of trimethylene carbonate. In one aspect, the initiator is a triol. In another aspect, the initiator is trimethylolpropane. In one aspect, the trimethylolpropane is used to initiate polymerization of trimethylene carbonate. Once the polymerization has essentially completed, lactide is added to the reaction mixture to produce a triaxial or linear polymer with a trimethylene carbonate based core that is terminated with a block of polylactide.

In another aspect, the polymer comprises polydioxanone. In another aspect, the polymer comprises polylactic acid. In one aspect the polylactic acid can be synthesized from L-lactide, D-lactide, D,L-lactide or a combination thereof.

In one aspect, the polymer comprises a copolymer of residues of lactide, trimethylene carbonate and ε-caprolactone. In one aspect, the copolymer is a block copolymer. In one aspect, the block copolymer has one block of trimethylene carbonate residues and a second block comprising residues of lactide and ε-caprolactone residues. In one aspect, the copolymer can be manufactured with an added lactide monomer of at least 70% of the total weight of all added monomers. In a preferred aspect, the added lactide monomer is between 70% and 90% of the total weight of all added monomers. In one aspect, the copolymer can be manufactured with an added TMC monomer of at least 10% of the total weight of all added monomers. In a preferred aspect, the added TMC monomer is between 10% and 20% of the total weight of all added monomers. In one aspect, the copolymer can be manufactured with an added ε-caprolactone monomer of at least 3% of the total weight of all added monomers. In a preferred aspect, the added ε-caprolactone monomer is between 3% and 15% of the total weight of all added monomers. In one aspect, the initiator used for the polymerization is a hydroxyl-based initiator. In one aspect, the initiator is a diol. In another aspect, the initiator is 1,3 propanediol. In one aspect, the 1,3 propanediol is used to initiate polymerization of trimethylene carbonate. In one aspect, the initiator is a triol. In another aspect, the initiator is trimethylolpropane. In one aspect, the trimethylolpropane is used to initiate polymerization of trimethylene carbonate. Once the polymerization has essentially completed, lactide and e-caprolactone are added to the reaction mixture to produce a triaxial or linear polymer with a poly(trimethylene carbonate) based core that is terminated with a block of lactide-co-caprolactone copolymer.

In one aspect, the polymer comprises a copolymer of residues of glycolide, trimethylene carbonate and ε-caprolactone. In one aspect, the copolymer is a block copolymer. In one aspect, the block copolymer has one block of trimethylene carbonate residues and a second block comprising residues of glycolide and ε-caprolactone. In one aspect, the copolymer can be manufactured with an added glycolide monomer of at least 45% of the total weight of all added monomers. In a preferred aspect, the added glycolide monomer is between 45% and 65% of the total weight of all added monomers. In one aspect, the copolymer can be manufactured with an added TMC monomer of at least 20% of the total weight of all added monomers. In a preferred aspect, the added TMC monomer is between 20% and 30% of the total weight of all added monomers. In one aspect, the copolymer can be manufactured with an added ε-caprolactone monomer of at least 15% of the total weight of all added monomers. In a preferred aspect, the added ε-caprolactone monomer is between 15% and 30% of the total weight of all added monomers. In one aspect, the initiator used for the polymerization is a hydroxyl-based initiator. In one aspect, the initiator is a triol. In another aspect, the initiator is trimethylolpropane. In one aspect, the trimethylolpropane is used to initiate a polymerization of monomers minimally comprising trimethylene carbonate. Once the polymerization has essentially completed, monomers minimally comprising glycolide are added to the reaction mixture to produce a triaxial polymer with a trimethylene carbonate based core that is terminated with a homopolymer or copolymer block of a glycolide-based end graft.

In one aspect, the polymer comprises a copolymer of residues of lactide, trimethylene carbonate and ε-caprolactone. Optionally, the polymer may include glycolide. In one aspect, the copolymer is a block copolymer. In one aspect, the block copolymer has one block of poly(trimethylene carbonate) and a second block comprising residues of lactide. In one aspect, the copolymer can be manufactured with an added lactide monomer of at least 35% of the total weight of all added monomers. In a preferred aspect, the added lactide monomer is between 30% and 45% of the total weight of all added monomers. In one aspect, the copolymer can be manufactured with an added TMC monomer of at least 10% of the total weight of all added monomers. In a preferred aspect, the added TMC monomer is between 10% and 40% of the total weight of all added monomers. In one aspect, the copolymer can be manufactured with an added ε-caprolactone monomer of at least 30% of the total weight of all added monomers. In a preferred aspect, the added ε-caprolactone monomer is between 30% and 40% of the total weight of all added monomers. In one aspect, the initiator used for the polymerization is a hydroxyl-based initiator. In one aspect, the initiator is a triol. In another aspect, the initiator is trimethylolpropane or triethanolamine. In one aspect, the trimethanolamine is used to initiate a polymerization of monomers minimally comprising trimethylene carbonate. Once the polymerization has essentially completed, monomers minimally comprising lactide are added to the reaction mixture to produce a triaxial polymer with a poly(trimethylene carbonate) based core that is terminated with a homopolymer or copolymer block of a lactide-based end graft.

Polyesters can include polyhydroxyalkanoates. Examples of polyhydroxyalkanoates include but are not limited to poly(3-hydroxybutyrate) (PHB), poly(3-hydroxybutyrate-co-3-hydroxyvalerate) (PHBV), Poly(3-hydroxybutyrate-co-4-hydroxybutyrate) (P(3HB-co-4HB)), Poly[3-hydroxybutyrate-co-3-hydroxyhexanoate] (P(3HB-co-3HH)), and Poly[(R)-4-hydroxybutyrate] poly(4-hydroxybutyrate) (P(4HB).

In one aspect, the polymer comprises a multiblock copolymer containing blocks of poly(lactide-co-trimethylene carbonate) and blocks of poly(lactide-co-glycolide). In an aspect, copolymer of lactide/glycolide has a lactide/glycolide mole ratio of 60-90/40-10. In an aspect, blocks of poly(lactide-co-glycolide) have a lactide/glycolide mole ratio of 60-90/40-10. In an aspect, the polymer comprises a segmented, aliphatic polyurethane comprising polyoxyalkylene glycol chains covalently linked to polyester or poly(ester-carbonate) chain segments, interlinked with aliphatic urethane segments. The polyoxyalkylene glycol chains comprise at least one type of oxyalkylene sequences selected from the group represented by oxyethylene, oxypropylene, oxytrimethylene, and oxytetramethylene repeat units. In certain embodiments, the polyoxyalkylene glycol chain has an average molecular weight of 200-1200 dalton. In other embodiments, the polyoxyalkylene glycol chain is PEG 200, PEG 300, PEG 400, PEG 500, PEG 600, PEG 700, PEG 800, PEG 900, PEG 1000, and derivatives thereof. The polyester or poly(ester-carbonate) chain segments are derived from at least one cyclic monomer selected from the group represented by .epsilon.-caprolactone, trimethylene carbonate, p-dioxanone, 1,5-dioxepan-2-one, l-lactide, dl-lactide, glycolide, morpholinedione, or morpholine-2,5-dione, and combinations thereof. The aliphatic urethane segments are derived from at least one diisocyanate selected from the group consisting of hexamethylene diisocyanate, lysine-derived diisocyanate, and cyclohexane bis(methylene isocyanate).

In an aspect, the segmented, aliphatic polyurethane has an ether/ester mass ratios of 20-49/80-51, preferably 25-40/75-55 and, most preferably 30-40/70-60. In another aspect, the segmented, aliphatic polyurethane has a prepolymer/diisocyante mass ratio in the range of 1:0.5 to 1:1.4. In an aspect, the segmented, aliphatic polyurethane has a prepolymer/diisocyante mass ratio of 1:0.66, 1:0.8 or 1:1.2.

In an aspect, the polymer comprises an absorbable, low crystallinity, segmented block copolymer, wherein the copolymer is a polyaxial copolyester made from trimethylene carbonate and at least one cyclic monomer selected from the group consisting of p-dioxanone, 1,5-dioxapan-2-one, glycolide, l-lactide, .epsilon.-caprolactone, and a morpholinedione, or morpholine-2,5-dione. In an aspect, the first block is prepared by the reaction of glycolide, trimethylene carbonate and ε-caprolactone using triethanolamine as the initiator and tin (II) 2-ethylhexanoate as the catalyst. In an aspect the second block comprises residues of lactide and glycolide. In an aspect, the block copolymer is prepared from the first block and then reacting the lactide and glycolide in the presence of the prepared first block. In an aspect, the block copolymer comprises residues of glycolide, trimethylene carbonate, ε-caprolactone and lactide.

The polymer may further comprise a solvent. In an aspect, the solvent is water soluble. In an aspect, the solvent is selected from the group consisting of N-methyl-2-pyrrolidone (NMP), polyethylene glycol (PEG), dimethyl sulfoxide (DMSO). In an aspect, the polymer: solvent ratio (w/w) is about 1:0.05 to about 1:3. In another aspect, the polymer to solvent ratio (w/w) is about 1:0.05 to about 1:1.

A containment layer or a medical device and/or composition may be made from a base polymer that is amorphous, compliant and elastomeric. It can also be crystallizable, but too much crystallinity may reduce the compliant nature of a polymer. If a higher crystalline material is chosen for use, then it may be advisable to incorporate a plasticizer such as PEG into the layer in order to reduce the final crystallinity of the layer when applied to the medical device and/or composition. As mentioned above, the polymer can be polyaxial or linear, blocked or segmented or random. For a flexible and compliant containment layer, the organic polymer(s) may be minimally crystallinity or may be amorphous.

The organic polymer may be prepared from a prepolymer and end-graft(s) if it is a block copolymer, or it may not be prepared from a prepolymer. In one aspect, one or more monomers selected from caprolactone, trimethylene carbonate, and/or l-lactide are used to form the organic polymer for the containment layer in order to extend the degradation time frame beyond that of the medical device and/or composition.

Suitable degradable organic polymers other than polyesters include polyether-esters, polyether-ester-urethanes (bioabsorbable urethanes), polyether-urethanes and polyether-urethane-ureas, the latter examples being very slowly and typically incompletely degradable.

In various aspects, a degradable medical device and/or composition and/or a containment layer is made from any of the following polymers. A polymer comprising greater than about 65% glycolide in end-graft, that is a semicrystalline, polyaxial block copolyester, prepared in a two-step reaction from an amorphous prepolymer and crystalline end graft. A polymer that is greater than about 80% glycolide, is a semicrystalline, polyaxial block copolyester, prepared in a two-step reaction from an amorphous prepolymer and crystalline end graft. A semicrystalline, polyaxial segmented copolyester prepared in a single step reaction (no prepolymer is used). A semicrystalline, linear block copolyester, prepared in a two-step reaction from an amorphous prepolymer and crystalline end graft. A triblock copolymer with crystalline end grafts. A diblock copolymer. A semicrystalline, linear segmented copolyester prepared in a single step reaction (i.e., no prepolymer is used). A polymer having an inherent viscosity of greater than 1.0, having a crystallizable end graft. A polyaxial block copolymer. A polymer prepared from an amorphous prepolymer and amorphous end graft. A linear block copolymer (triblock, diblock, pentablock, etc.). A linear, segmented copolymer. A linear random copolymer which is amorphous and thus is both compliant and flexible. The foregoing are exemplary only of the organic polymers that may be used to prepare a suitable degradable medical device and/or composition, component thereof, or containment layer.

Another suitable polymer is a mixture comprising (a) a bioerodible polyester network formed by reaction between reactive species that comprise a polyol and a polycarboxylate, wherein at least one of the polyol and polycarboxylate has a functionality of three or more, and (b) a bioerodible thermoplastic polymer. Optionally, one or more of the following may further characterize these compositions: the polyol is selected from a non-polymeric diol, a polymeric diol, a non-polymeric triol, a polymeric triol; the polycarboxylate is selected from a non-polymeric dicarboxylate, a polymeric dicarboxylate, a non-polymeric tricarboxylate, and a polymeric tricarboxylate; the reactive species comprise a triol, a tricarboxylate, or both; the reactive species comprise (a) non-polymeric tricarboxylate and (b) a polyester polyol; the reactive species comprise (a) citric acid and (b) a polycaprolactone diol, a polycaprolactone triol or both; the bioerodible thermoplastic polymer has a melting point above body temperature; the bioerodible thermoplastic polymer has a glass transition temperature below room temperature; and the bioerodible thermoplastic polymer is a bioerodible thermoplastic polyester. See, e.g., U.S. Patent Publication No. 20160166739.

A polymer used to make a degradable medical device and/or composition or component, such as a coating, containment layer, may be a polymer capable of use in additive manufacturing methods, e.g., 3D printing. Such degradable polymers for additive manufacture are known in the art and are useful for medical devices and/or compositions, and/or coatings or containment layers described herein.

In an aspect, a medical device and/or composition of the present disclosure may include a containment layer in addition to a medical device and/or composition. The location of the containment layer relative to the medical device and/or composition, and the properties of the containment layer in terms of physical and chemical properties, both assist in managing the degradation and/or elimination of the degradable medical device and/or composition from the subject. In particular, the containment layer serves, in part, to manage the degradation and/or elimination of the medical device and/or composition from the subject. The properties of the containment layer should also be selected with a view to managing the degradation and/or elimination of the containment layer itself from the subject.

In an aspect, degradable medical devices and/or compositions of the present disclosure are degradable to at least some extent. In other words, the degradable medical device and/or composition will degrade when placed into or on the subject. That degradation may be a physical or chemical degradation. Physical degradation refers to a change in the physical or mechanical properties of the medical device and/or composition. For example, the device may break down into pieces, and thus lose its integrity. As another example, the device may soften and become compliant. As yet another example, the device may absorb fluid and swell. In each of these cases, the device undergoes a change in physical or mechanical properties. Chemical degradation refers to a change in chemical composition. For example, an organic polymer from which the device is made may undergo hydrolytic bond cleavage or enzymatically-induced bond cleavage, and thereby lose molecular weight. As another example, water-soluble components of the medical device and/or composition may dissolve in water and leave the vicinity of the medical device and/or composition. In each of these examples, the chemical degradation produces a change in the chemical description of the medical device and/or composition. It may be the case that degradation of the medical device and/or composition simultaneously achieves physical as well as chemical degradation. In any event, the containment layer of the present disclosure may serve, in part or entirely, to influence this degradation. Thus, the properties of containment layer can be used to manage the degradation and/or elimination of the medical device and/or composition from the subject.

In an aspect, a containment layer may provide a physical barrier between the tissue of a subject and the medical device and/or composition. Such a barrier is useful, for example, when the device degrades by breaking into pieces and it is desired to manage the dispersement or dissemination of those pieces. For example, in an aspect, a containment layer may be relatively long-lasting compared to the medical device and/or composition, so that as the medical devices and/or compositions is breaking into pieces, the containment layer is maintaining sufficient structural integrity so that those pieces are retained within the containment layer. Such a containment layer is useful when a portion of all of a medical device and/or composition is within the kidney and it is undesirable that pieces from the medical device and/or composition should contact the inside of the kidney and calcify. A containment layer may effectively restrict the movement of pieces of the medical device and/or composition.

In an aspect, the containment layer provides a physical or chemical barrier between the degradation-inducing fluids of the subject and the medical device and/or composition. This layer can be used to influence the spatial and temporal degradation of the medical device and/or composition. For example, in one aspect the containment layer is a discontinuous layer such that the layer covers some but not all of the medical device and/or composition. In this situation, the containment layer effectively acts as a barrier between the medical device and/or composition and the degradation-causing fluid of the subject, which restricts contact between the medical device and/or composition and the fluid. The containment layer thereby allows the exposed portion(s) of the medical device and/or composition to degrade more quickly than will the nonexposed portion(s) of the medical device and/or composition. In this way, the containment layer is used to manage in which sites the device will initially degrade.

In an aspect, a graduated containment layer is used to manage the spatial and temporal degradation and/or elimination of the medical device and/or composition. For example, coating layer may serve as a containment layer, and a medical device and/or composition may have a single coating layer over a first portion of the device, a double coating layer over a second portion of the device, and a triple coating layer over a third portion of the device. Assuming the composition of the coating layer is the same at each location, the first portion of the medical device and/or composition will begin to degrade before the second and third portions of the device. Depending on the relative thicknesses at each location, the first portion may significantly degrade and be eliminated from the subject, while the second and third portions of the device are still significantly intact. The degradation and elimination of the first portion of the medical device and/or composition will allow increased access of biological fluid to the second portion of the medical device and/or composition, with the result that the second portion will undergo degradation even though the second portion may still be covered by the coating (i.e., containment layer). The second portion of the device will undergo degradation and elimination, followed by degradation and elimination of the third portion of the device. In this example, the containment layer (coating) manages the rate at which various portions of a medical device and/or composition degrade and are eliminated from the subject. The containment layer may also function to manage the dispersement or dissemination of those pieces, i.e., to restrict the movement of those pieces within the subject.

Degradable medical devices and/or compositions, and components thereof, may include one or more active agents. The amount of the active agent incorporated into a degradable medical device and/or composition will depend on the nature of the degradable medical device and/or composition, the active agent, the condition of the subject, and so forth. The amount may be readily determined by those of ordinary skill in the art. Degradable medical devices and/or compositions of the present disclosure generally include at least one pharmaceutical or medicinal compound or molecule, referred to herein as an active agent. An active agent may also be referred to as an active pharmaceutical ingredient (API) or a drug. As noted previously, an active agent refers to one, as well as more than one biologically active agent. An active agent may be described in terms of its biological function or its chemical class. An exemplary active agent includes, but is not limited to, a/an antiandrogen, antibacterial, antioestrogen, androgen or anabolic agent, antibiotic, antimigraine drug, antihistamine, antianxiety drug, antidiuretic, antihistamine, antirheumatoid agent, antigen, analgesic, antidepressant, antiinflammatory, anesthetic, aminoglycoside, antibody, antibody fragment, antiviral, adrenergic stimulant, anticonvulsant, antiangina agent, antiarrhyrthmic, antimalarial, anti-mitotic agent, anthelmintic, anoretic agent, antitussive, antipruritic, antipyretic, anti-Alzheimer's agent, anti-Parkinson's agent, antiemetic and antinauseant, antihypertensive, anticoagulant, antifungal, antimicrobial, allergen, antidiarrheal, antihyperuricaemia agent, adrenergic stimulant, antiparasitic agent, antiproliferative agent, antipsychotic drug, antithyroid agent, beta-adrenergic blocking agent, bronchodilator, bronchospasm relaxant, blood clotting factor, blood coagulation factor, cytotoxic agent, cytostatic agent, chemotherapeutic, clot inhibitor, clot dissolving agent, cell, CNS stimulant, corticosteroid, calcium channel blocker, cofactor, ceramide, cardiotonic glycoside, cytokine (e.g., lymphokine, monokine, chemokine), colony stimulating factor (e.g., GCSF, GM-CSF, MCSF), dermatological agent, decongestant, diuretic, expectorant, endectocide agent, growth factor, growth factor receptor, growth factor receptor inhibitor, hemostatic agent, hypoglycemic agent, hormone or hormone analog, hypercalcemia, hypnotic, interleukin (IL-2, IL-3, IL-4, IL-6); interferon (e.g., β-IFN, α-IFN and γ-IFN), immunosuppressant, muscle relaxant, microorganism, non-steroidal anti-inflammatory agent, nucleic acid, nutritional agent, neuromuscular blocking agent, neuroleptic, neurotoxin, nutraceutical, oligonucleotide, oestrogen, obstetric drug, ovulation inducer, opioid, opioid agonist or antagonist progestogen, pituitary hormone, pituitary inhibitor protein, peptide, polysaccharide, protease inhibitor, prostaglandin, quinolone, reductase inhibitor, sulfa drug, sclerosant, sedative, sodium channel blockers, steroid, steroidal anti-inflammatory agent, smoking cessation agent, toxin, thrombolytic agent, thyroid hormone, tumor necrosis factor; vesicle, vitamin, mineral, virus, vasodilator, or a vaccine. Exemplary active agents include the following options, and PCT Patent Application Serial No. PCT/US2020/022241, filed Mar. 12, 2020, is herein incorporated in its entirety for its teaching of compositions comprising active agents.

In various aspects of the present disclosure, a medical device and/or composition may release one or more active agents where representative examples of active agents include, but are not limited to, one or more suitable members of the following: alpha-adrenergic blockers, analgesic agents, anti-cancer agents, antineoplastic agents, anti-inflammatory agents, anti-microbial agents, antiproliferative agents, anti-spasmodic agents, beta-adrenergic agonists, bronchodilators (e.g., for muscle relaxant properties), calcium channel blockers, corticosteroids, anesthetic agents, narcotic analgesic agents, nitric oxide donors, nitric oxide releasing compounds, non-narcotic analgesic agents, prostaglandins, and among others, as well as combinations thereof.

Additional representative examples of active agents include, but are not limited to, one or more of the following: Angiogenesis Inhibitors, 5-Lipoxygenase Inhibitors and Antagonists, cellular receptor inhibitors, Chemokine Receptor Antagonists CCR (1, 3, and 5), Cell Cycle Inhibitors, Cyclin Dependent Protein Kinase Inhibitors, EGF (Epidermal Growth Factor) Receptor Kinase Inhibitors, Elastase Inhibitors, Factor Xa Inhibitors, Farnesyltransferase Inhibitors, Fibrinogen Antagonists, agonists, molecules that inhibit or stimulate receptors or antigens, such as antibodies or antibody fragments, Guanylate Cyclase Stimulants, Heat Shock Protein 90 Antagonists, HMGCoA Reductase Inhibitors, Hydroorotate Dehydrogenase Inhibitors, IKK2 Inhibitors, IL-1, ICE and IRAK Antagonists, IL-4 Agonists, Immunomodulatory Agents, Inosine monophosphate dehydrogenase inhibitors, Leukotriene Inhibitors, MCP-1 Antagonists, MMP Inhibitors, NF kappa B Inhibitors, NO Agonists, P38 MAP Kinase Inhibitors, Phosphodiesterase Inhibitors, TGF beta Inhibitors, TNF alpha Antagonists and TACE Inhibitors, Tyrosine Kinase Inhibitors, Vitronectin Inhibitors, Fibroblast Growth Factor Inhibitors, Protein Kinase Inhibitors, PDGF Receptor Kinase Inhibitors, Endothelial Growth Factor Receptor Kinase Inhibitors, Retinoic Acid Receptor Antagonists, Platelet Derived Growth Factor Receptor Kinase Inhibitors, Fibronogin Antagonists, Antimycotic Agents, Bisphosphonates, Phospholipase A1 Inhibitors, Histamine H1/H2/H3 Receptor Antagonists, Macrolide Antibiotics, GPIIb IIIa Receptor Antagonists, Endothelin Receptor Antagonists, Peroxisome Proliferator-Activated Receptor Agonists, Estrogen Receptor Agents, Somatostatin Analogues, Neurokinin 1 Antagonists, Neurokinin 3 Antagonist, Neurokinin Antagonist, VLA-4 Antagonist, Osteoclast Inhibitor, DNA topoisomerase ATP Hydrolysing Inhibitor, Angiotensin I Converting Enzyme Inhibitor, Angiotensin II Antagonist, Enkephalinase Inhibitor, Peroxisome Proliferator-Activated Receptor Gamma Agonist Insulin Sensitizer, Protein Kinase C Inhibitor, CXCR3 Inhibitors, Itk Inhibitors, Cytosolic phospholipase A2-alpha Inhibitors, PPAR Agonist, Immunosuppressants, Erb Inhibitor, Apoptosis Agonist, Lipocortin Agonist, VCAM-1 antagonist, Collagen Antagonist, Alpha 2 Integrin Antagonist, TNF Alpha Inhibitor, Nitric Oxide Inhibitor, and Cathepsin Inhibitor.

Examples of alpha-adrenergic blocker active agents include, but are not limited to: alfuzosin, amosulalol, arotinilol, dapiprazole, doxazosin, ergoloid, fenspiride, idazoxan, indoramin, labetalol, manotepil, mesylates, naftopidil, nicergoline, prazosin, tamsulosin, terazosin, tolazoline, trimazosin, and yohimbine.

Examples of anesthetic agents include, but are not limited to: benzocaine, cocaine, lidocaine, mepivacaine, and novacaine.

Examples of beta-adrenergic agonists include, but are not limited to: albuterol, bambuterol, bitolterol, carbuterol, clenbuterol, clorprenaline, denopamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, salmerterol, soterenol, terbutaline, tretoquinol, tulobuterol and xamoterol.

Examples of anti-cancer, anti-proliferative and antineoplastic agents include, but are not limited to: agents affecting microtubule dynamics (e.g., colchicine, Epo D, epothilone, paclitaxel, vinblastine, vincristine, etc.), alkyl sulfonates, angiogenesis inhibitors (e.g., angiostatin, endostatin, squalamine, etc.), antimetabolites such as purine analogs (e.g., 6-mercaptopurine or cladribine, which is a chlorinated purine nucleoside analog, etc.), pyrimidine analogs (e.g., 5-fluorouracil, cytarabine, etc.) and antibiotics (e.g., daunorubicin, doxorubicin, etc.), caspase activators, cerivastatin, cisplatin, Epirubicin, ethylenimines, flavopiridol, limus family active agents (e.g., everolimus, sirolimus, tacrolimus, zotarolimus, etc.), methotrexate, nitrogen mustards, nitrosoureas, proteasome inhibitors, and suramin.

An active agent may comprise growth factor (e.g. VEGF, FGF) antagonists. An active agent may comprise growth factor receptor inhibitor. An active agent may comprise an FGF receptor (FGFR) inhibitor. Such active agents may be useful for treating one or more types of cancer. For example, urothelial cancer, most frequently in the bladder, is the sixth most common type of cancer in the U.S. It is estimated that in 2018, 81,190 new cases of bladder cancer will be diagnosed in the U.S. and an estimated 17,240 bladder cancer deaths will occur. The relative five-year survival rate for patients with Stage IV metastatic bladder cancer is currently five percent. Patients with metastatic urothelial cancer, who have FGFR genetic alterations, have poor prognoses and a high unmet need based on low response rates and may be resistant to treatment with immune-checkpoint inhibitors. Erdafitinib, C₂₅H₃₀N₆O₂, which may also be known as 1,2-Ethanediamine, N1-(3,5-dimethoxyphenyl)-N2-(1-methylethyl)-N1-(3-(1-methyl-1H-pyrazol-4-yl)-6-quinoxalinyl)-is an investigational, once-daily oral pan-fibroblast growth factor receptor (FGFR) inhibitor being studied in Phase 2 and Phase 3 clinical trials for the treatment of patients with locally advanced or metastatic urothelial cancer. FGFRs are a family of receptor tyrosine kinases, which can be activated by genetic alterations in a variety of tumor types, and these alterations may lead to increased tumor cell growth and survival. FGFRs are a subset of tyrosine kinases which are unregulated in some tumors and influence tumor cell differentiation, proliferation, angiogenesis, and cell survival. Erdafitinib is being evaluated for safety and efficacy in phase II clinical trials for cholangiocarcinoma, gastric cancer, non-small cell lung cancer, and esophageal cancer.

Methods disclosed herein comprise administering an effective amount of an active agent via a degradable medical device and/or composition disclosed herein for treatment of chronic or acute diseases, or pathological conditions, for diagnostic purposes, for maintenance purposes or for other conditions of a subject. Those of skill in the art can understand the disclosed devices and compositions, in conjunction with active agents, can be used for treatment, prevention, diagnosis, maintenance, and euthanistic methods for subjects. Examples provided herein are for illustration and are not intended to be limiting.

For example, a method of treatment disclosed herein comprises administering an effective amount of an active agent via a degradable medical device and/or composition disclosed herein for treatment of urothelial cancer, bladder cancer, cholangiocarcinoma, gastric cancer, non-small cell lung cancer, and esophageal cancer. For example, a degradable medical device and/or composition may comprise erdafitinib, C₂₅H₃₀N₆O₂, as an active agent. In an aspect, a degradable medical device and/or composition disclosed herein may comprise erdafitinib and a polymer, disclosed herein. Degradable medical devices and/or compositions may comprise derivatives of erdafitinib, salts of erdfitinib, pharmaceutical diluents or excipients, and other known additives in formulations comprising erdafitinib. Other FGFR inhibitors that can be used include infigratinib (BGJ398), PRN1371 (Principica Biopharma) and AZD4547 (AstraZeneca). Other kinase inhibitors that can be used include imatinib, ponatinib, pazopanib and trametinib.

Examples of antimicrobial agents include, but are not limited to: benzalkonium chlorides, chlorhexidine, nitrofurazone, silver particles, silver salts, metallic silver and antibiotics, such as gentamicin, minocycline and rifampin, triclosan.

Examples of bronchodilators include, but are not limited to: (a) ephedrine derivatives such as albuterol, bambuterol, bitolterol, carbuterol, clenbuterol, clorprenaline, dioxethedrine, ephedrine, epinephrine, eprozinol, etafedrine, ethylnorepinephrine, fenoterol, formoterol, hexoprenaline, isoetharine, isoproterenol, mabuterol, metaproterenol, n-methylephedrine, pirbuterol, procaterol, protokylol, reproterol, rimiterol, salmeterol, soterenol, terbutaline and tulobuterol, (b) quaternary ammonium compounds such as bevonium methyl sulfate, flutropium bromide, ipratropium bromide, oxitropium bromide and tiotropium bromide, (c) xanthine derivatives such as acefylline, acefylline piperazine, ambuphylline, aminophylline, bamifylline, choline theophyllinate, doxofylline, dyphylline, etamiphyllin, etofylline, guaithylline, proxyphylline, theobromine, 1-theobromineacetic acid and theophylline, and (d) other bronchodilators such as fenspiride, medibazine, methoxyphenanime and tretoquinol, among others, as well as combinations and pharmaceutically acceptable salts, esters and other derivatives of the forgoing.

Examples of calcium channel blockers include, but are not limited to: arylalkylamines (including phenylalkylamines) such as bepridil, clentiazen, fendiline, gallopamil, mibefradil, prenylamine, semotiadil, terodiline and verapamil, benzothiazepines such as diltiazem; calcium channel blockers such as bencyclane, etafenone, fantofarone, monatepil and perhexiline, among other calcium channel blockers; dihydropyridine derivatives (including 1,4-dihydropyridine derivatives) such as amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine and nitrendipine, piperazine derivatives such as cinnarizine, dotarizine, flunarizine, lidoflazine and lomerizine.

Examples of corticosteroids include, but are not limited to: betamethasone, cortisone, deflazacort, dexamethasone, hydrocortisone, methylprednisolone, prednisolone, prednisone and triamcinolone, among others, as well as combinations and pharmaceutically acceptable salts, esters and other derivatives of the same.

Examples of nitric oxide donors/releasing molecules include, but are not limited to: inorganic nitrates/nitrites such as amyl nitrite, isosorbide dinitrate and nitroglycerin, inorganic nitroso compounds such as sodium nitroprusside, sydnonimines such as linsidomine and molsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of, natural polymers/oligomers, oligosaccharides, peptides, polysaccharides, proteins, and synthetic polymers/oligomers), as well as C-nitroso-compounds, L-arginine, N-nitroso-compounds, and O-nitroso-compounds.

Examples of prostaglandins and analogs thereof for use in the present disclosure include, but are not limited to: prostaglandins such as PGE1 and PGI2 and prostacyclin analogs such as beraprost, carbacyclin, ciprostene, epoprostenol, and iloprost.

Examples of narcotic analgesic agents include, but are not limited to: codeine, fentanyl, hydromorphonein, levorphanol, meperidine, methadone, morphine, oxycodone, oxymorphone, propoxyphene, and pentazocine, among others, as well as combinations and pharmaceutically acceptable salts, esters and other derivatives of the same.

Examples of non-narcotic analgesic agents include, but are not limited to: analgesic agents such as acetaminophen, and non-steroidal anti-inflammatory active agents such as aspirin, celecoxib, diflunisal, diclofenac, etodolac, fenoprofen, flurbiprofen, ibuprofen, ketoprofen, ketorolac, meclofenamate, meloxicam, nabumetone, naproxen, naproxen indomethacin, oxaprozin, piroxicam, salsalate, sulindac, tolmetin, and valdecoxib.

An active agent may comprise antidiarrheals such as diphenoxylate, loperamide and hyoscyamine. An active agent may comprise antihypertensives such as hydralazine, minoxidil, captopril, enalapril, clonidine, prazosin, debrisoquine, diazoxide, guanethidine, methyldopa, reserpine, trimethaphan. An active agent may comprise calcium channel blockers such as diltiazem, felodipine, amlodipine, nitrendipine, nifedipine and verapamil. An active agent may comprise antiarrhyrthmics such as amiodarone, flecainide, disopyramide, procainamide, mexiletene and quinidine. An active agent may comprise antiangina agents such as glyceryl trinitrate, erythrityl tetranitrate, pentaerythritol tetranitrate, mannitol hexanitrate, perhexilene, isosorbide dinitrate and nicorandil. An active agent may comprise beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

An active agent may comprise cardiotonic glycosides such as digoxin and other cardiac glycosides and theophylline derivatives. An active agent may comprise adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine. An active agent may comprise vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol. An active agent may comprise antiproliferative agents such as paclitaxel, estradiol, actinomycin D, sirolimus, tacrolimus, everolimus, 5-fluorouracil, Gemcitabine and dexamethasone.

An active agent may comprise antimigraine preparations such as ergotanmine, dihydroergotamine, methysergide, pizotifen and sumatriptan. An active agent may comprise anticoagulants and thrombolytic agents such as warfarin, dicoumarol, low molecular weight heparins such as enoxaparin, streptokinase and its active derivatives. An active agent may comprise hemostatic agents such as aprotinin, tranexamic acid and protamine.

An active agent may comprise analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine, dihydrocodeine; acetylsalicylic acid (aspirin), paracetamol, synthetic alpha2-adrenoreceptor agonist, dexmedetomidine hydrochloride, flunixin meglumine, meperidine, phenylbutazone and phenazone. An active agent may include an agonist or antagonist of a known opioid compound.

An active agent may comprise immunosuppressants, antiproliferatives and cytostatic agents such as rapamycin (sirolimus) and its analogs (everolimus and tacrolimus). An active agent may comprise neurotoxins such as capsaicin and botulinum toxin (botox). An active agent may comprise hypnotics and sedatives such as the barbiturates amylobarbitone, butobarbitone and pentobarbitone and other hypnotics and sedatives such as chloral hydrate, chlormethiazole, hydroxyzine and meprobamate. An active agent may not comprise disulfram. An active agent may comprise disulfram. An active agent may comprise antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam. An active agent may comprise copmounds effective in treating addiction, including but not limited to, acamprosate, topiramate, naltrexone, or nalmefene. An active agent may comprise BSA (bovine serum albumin).

An active agent may comprise neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium. An active agent may comprise antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline. An active agent may comprise central nervous system (CNS) stimulants such as caffeine and 3-(2-aminobutyl) indole.

An active agent may comprise antipruritics such as synthetic Janus Kinase (JAK) inhibitors, NK-1 receptor antagonists, antibodies that neutralize interleukin-31 (IL-31). These can include oclacitinib maleate, Serlopitant and Lokivetmab. An active agent may comprise anti-Alzheimer's agents such as tacrine. An active agent may comprise anti-Parkinson's agents such as amantadine, benserazide, carbidopa, levodopa, benztropine, biperiden, benzhexol, procyclidine and dopamine-2 agonists such as S (−)-2-(N-propyl-N-2-thienylethylamino)-5-hydroxytetralin (N-0923). An active agent may comprise anticonvulsants such as phenytoin, valproic acid, primidone, phenobarbitone, methylphenobarbitone and carbamazepine, ethosuximide, methsuximide, phensuximide, sulthiame and clonazepam.

An active agent may comprise antiemetics and antinauseants such as the phenothiazines prochloperazine, thiethylperazine, a neurokinin (NK1) receptor antagonist, maropitant citrate and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride. An active agent may comprise non-steroidal anti-inflammatory agents including their racemic mixtures or individual enantiomers where applicable, preferably which can be formulated in combination with dermal and/or mucosal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, 6-chloro-α-methyl-9H-carbazole-2-acetic acid (carprofen), flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

An active agent may comprise antirheumatoid agents such as penicillamine, aurothioglucose, sodium aurothiomalate, methotrexate and auranofin. An active agent may comprise muscle relaxants such as baclofen, diazepam, cyclobenzaprine hydrochloride, dantrolene, methocarbamol, orphenadrine and quinine. An active agent may comprise agents used to treat gout and hyperuricaemia such as allopurinol, colchicine, probenecid and sulphinpyrazone. An active agent may comprise oestrogens such as estradiol, estriol, estrone, ethinylestradiol, mestranol, stilbestrol, dienestrol, epiestriol, estropipate and zeranol.

An active agent may comprise progesterone and other progestagens such as allylestrenol, dydrgesterone, lynestrenol, norgestrel, norethyndrel, norethisterone, norethisterone acetate, gestodene, levonorgestrel, medroxyprogesterone and megestrol. An active agent may comprise antiandrogens such as cyproterone acetate and danazol. An active agent may comprise antioestrogens such as tamoxifen and epitiostanol and the aromatase inhibitors, exemestane and 4-hydroxy-androstenedione and its derivatives. An active agent may comprise androgens and anabolic agents such as testosterone, methyltestosterone, clostebol acetate, drostanolone, furazabol, nandrolone oxandrolone, stanozolol, trenbolone acetate, dihydro-testosterone, 17-(α-methyl-19-noriestosterone and fluoxymesterone.

An active agent may comprise 5-α Reductase inhibitors such as finasteride, turosteride, LY-191704 and MK-306. An active agent may comprise corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone and triamcinolone acetonide.

An active agent may comprise glycosylated proteins, proteoglycans, and glycosaminoglycans such as chondroitin sulfate; chitin, acetyl-glucosamine and hyaluronic acid. An active agent may comprise complex carbohydrates such as glucans.

An active agent may comprise steroidal anti-inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

An active agent may comprise pituitary hormones and their active derivatives or analogs such as corticotrophin, thyrotropin, follicle stimulating hormone (FSH), a gonadotropin-releasing hormone (GnRH) analog, deslorelin acetate, cetrorelix acetate, gonadorelin acetate, clomiphene, human chorionic gonadotropin (HCG), luteinizing hormone (LH) and gonadotrophin releasing hormone (GnRH).

An active agent may comprise hypoglycemic agents such as insulin, chlorpropamide, glibenclamide, gliclazide, glipizide, tolazamide, tolbutamide and metformin. An active agent may comprise thyroid hormones such as calcitonin, thyroxine and liothyronine and antithyroid agents such as carbimazole and propylthiouracil. An active agent may comprise hormone agents such as octreotide. An active agent may comprise pituitary inhibitors such as bromocriptine. An active agent may comprise ovulation inducers such as clomiphene.

An active agent may comprise diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene. An active agent may comprise antidiuretics such as desmopressin, lypressin and vasopressin including their active derivatives or analogs. An active agent may comprise obstetric drugs including agents acting on the uterus such as ergometrine, oxytocin and gemeprost. An active agent may comprise prostaglandins such as alprostadil (PGE1), prostacyclin (PGI2), dinoprost (prostaglandin F2-alpha) and misoprostol.

An active agent may comprise antimicrobials including the cephalosporins such as cephalexin, cefoxytin and cephalothin. An active agent may comprise penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin. An active agent may comprise tetracyclines such as minocycline, chlortetracycline, tetracycline, demeclocycline, doxycycline, methacycline and oxytetracycline and other tetracycline-type antibiotics. An active agent may comprise amnioglycoides such as amikacin, amikin sulfate, gentamicin, kanamycin, neomycin, netilmicin and tobramycin. An active agent may comprise rifampin, or antimicrobial peptide (AMP), specifically the synthetic peptide hLF(1-11).

An active agent may comprise antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. An active agent may comprise quinolones such as nalidixic acid, cinoxacin, ciprofloxacin, enoxacin and norfloxacin; Sulphonamides such as phthalysulphthiazole, sulfadoxine, sulphadiazine, sulphamethizole and sulphamethoxazole. An active agent may comprise sulphones such as dapsone.

An active agent may comprise antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; Lincomycin Hydrochloride, tricyclic tetrahydroquinoline antibacterial agents, 8-pyrazinyl-S-spiropyrimidinetrione-oxazinoquinoline derivatives, 3-spiropyrimidinetrione-quinoline derivatives, thiadiazol-spiropyrimidinetrione-quinoline derivatives, (2R,4S,4aS)-10-fluoro-2,4-dimethyl-8-(4-methyloxazol-2-yl)-2,4,4a,6-tetrahydro-1H,1′H-spiro[[1,4]oxazino[4,3-a]quinoline-5,5′-pyrimidine]-2′,4′,6′(-3H)-trione, (2R,4S,4aS)-9,10-difluoro-2,4-dimethyl-8-(3-methylisoxazol-5-yl)-2,4,4a,6-tetrahydro-1H,1′H-spiro[[1,4]oxazino[4,3-a]quinoline-5,5′-pyrimidine]-2′,-4′,6′(3H)-trione, (2R,4S,4aS)-10-fluoro-2,4-dimethyl-8-(oxazol-2-yl)-2,4,4a,6-tetrahydro-1H-,1′H-spiro[[1,4]oxazino[4,3-a]quinoline-5,5′-pyrimidine]-2′,4′,6′(3′H)-tri-one, (2R,4S,4aS)-9,10-difluoro-2,4-dimethyl-8-(2-methyloxazol-5-yl)-2,4,4a,6-t-etrahydro-1H,1′H-spiro[[1,4]oxazino[4,3-a]quinoline-5,5′-pyrimidine]-2′,4′-,6′(3′H)-trione, (2R,4S,4aS)-9,10-difluoro-2,4-dimethyl-8-(oxazol-4-yl)-2,4,4a,6-tetrahydr-o-1H,1′H-spiro[[1,4]oxazino[4,3-a]quinoline-5,5′-pyrimidine]-2′,4′,6′(3′H)-trione, (2R,4S,4aS)-9-fluoro-2,4-dimethyl-8-(4-methyloxazol-2-yl)-2,4,4a,6-tetrah-ydro-1H,1′H-spiro[[1,4]oxazino[4,3-a]quinoline-5,5′-pyrimidine]-2′,4′,6′(3-′H)-trione, (2R,4S,4aS)-9,10-difluoro-8-(4-(4-fluorophenyl)oxazol-5-yl)-2,4-dimethyl-2,4,4a,6-tetrahydro-1H,1′H-spiro[[1,4]oxazino[4,3-a]quinoline-5,5′-pyrimidine]-2′,4′,6′(3′H)-trione, (2S,4R,4aR)-2,4-dimethyl-8-(oxazol-5-yl)-2,4,4a,6-tetrahydro-1H,1′H-spiro-[[1,4]oxazino[4,3-a]quinoline-5,5′-pyrimidine]-2′,4′,6′(3′H)-trione, (2S,4R,4aR)-8-(4-ethyloxazol-2-yl)-9,10-difluoro-2,4-dimethyl-2,4,4a,6-te-trahydro-1H,1′H-spiro[[1,4]oxazino[4,3-a]quinoline-5,5′-pyrimidine]-2′,4′,-6′(3H)-trione, (2R,4S,4aS)-9,10-difluoro-2,4-dimethyl-8-(oxazol-2-yl)-2,4,4a,6-tetrahydr-o-1H,1′H-spiro[[1,4]oxazino[4,3-a]quinoline-5,5′-pyrimidine]-2′,4′,6′(3′H)-trione and benzoyl peroxide.

An active agent may comprise antituberculosis drugs such as ethambutol, isoniazid, pyrazinamide, rifampicin and clofazimine. An active agent may comprise antimalarials such as primaquine, pyrimethamine, chloroquine, hydroxychloroquine, quinine, mefloquine and halofantrine. An active agent may comprise compounds including Azithromycin, Aztreonam, Cefaclor, Cefadroxil, Cefazolin, Cefdinir, Cefepime Hydrochloride, (cefoperazone sodium, Ceftaroline fosamil, avibactam, Ceftazidime sodium, Ceftibuten, ceftiofur, Tazobactam, cefovecin sodium [(6R,7R)-7-[[(2Z)-(2-amino-4-thiazolyl)(methoxyimino)acetyl]amino]-8-oxo-3-[(2S)-tetrahydro-2-furanyl]-5-thia-1-azabicyclo[4.2.0]oct-2-ene-2-carboxylic acid, monosodium salt] Cefuroxime Axetil, Cefuroxime, Cephalexin, Chloramphenicol Sodium, Ciprofloxacin HCl, Clarithromycin, Clindamycin hydrochloride, Clindamycin Palmitate hydrochloride, Clindamycin phosphate, Dalbavancin Hydrochloride, Daptomycin, Demeclocycline hydrochloride, Dicloxacillin, Doripenem, Doxycycline, Doxycycline calcium, Doxycycline hyclate, Doxycycline monohydrate, Ertapenem sodium, Erythromycin, Erythromycin Ethylsuccinate, Erythromycin lactobionate, Erythromycin stearate, Erythromycin, Fosfomycin tromethamine, Gemifloxacin mesylate, Gentamicin Sulfate, Imipenem, Kanamycin, Levofloxacin, Lincomycin hydrochloride, Linezolid, Meropenem, Methenamine Hippurate, Metronidazole, Metronidazole, Micafungin sodium, Minocycline Hydrochloride, Minocycline, Moxifloxacin hydrochloride, Nafcillin, Nalidixic acid, Neomycin Sulfate, Nitrofurantoin, Norfloxacin, Ofloxacin, Oritavancin diphosphate, Oxacillin, Penicillin G, Penicillin G benzathine, Penicillin G Sodium, Penicillin V Potassium, Piperacillin Sodium, Polymyxin B Sulfate, Quinupristin, dalfopristin, Spectinomycin hydrochloride, Streptomycin, Sulfamethoxazole, Tedizolid Phosphate, Telavancin, Telithromycin, Tetracycline Hydrochloride, Ticarcillin disodium, Tigecycline, Tobramycin Sulfate, Tobramycin, Trimethoprim hydrochloride, tulathromycin and Vancomycin hydrochloride.

An active agent may comprise antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. An active agent may comprise anthelmintics such as mebendazole, thiabendazole, niclosamide, praziquantel, pyrantel embonate and diethylcarbamazine. An active agent may comprise cytotoxic agents such as plicamycin, cyclophosphamide, dacarbazine, fluorouracil and its prodrugs (described, for example, in International Journal of Pharmaceutics, 111, 223-233 (1994)), methotrexate, procarbazine, Gemcitabine, 6-mercaptopurine and mucophenolic acid.

An active agent may comprise anorectic and weight reducing agents including dexfenflurarnine, fenfluramine, diethylpropion, mazindol and phentermine. An active agent may comprise agents used in treating hypercalcaemia such as calcitriol, dihydrotachysterol and their active derivatives or analogs. An active agent may comprise antitussives such as ethylmorphine, dextromethorphan and pholcodine.

An active agent may comprise antiparasitic and endectocide agents such as moxidectin, Ivermectin, Niclosamide, Praziquantel, Pyrantel, Pyrvinium, Albendazole, Flubendazole, Mebendazole and Thiabendazole. An active agent may comprise expectorants such as carbolcysteine, bromihexine, emetine, quanifesin, ipecacuanha and saponins. An active agent may comprise decongestants such as phenylephrine, phenylpropanolamine and pseudoephedrine.

An active agent may comprise bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives.

An active agent may comprise antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

An active agent may comprise local anaesthetics such as benzocaine, bupivacaine, amethocaine, lignocaine, lidocaine, cocaine, cinchocaine, dibucaine, mepivacaine, prilocaine, etidocaine, veratridine (specific c-fiber blocker) and procaine. An active agent may comprise stratum corneum lipids such as ceramides, cholesterol and free fatty acids, for improved skin barrier repair (Man, et al. J. Invest. Dermatol., 106(5), 1096, (1996)). An active agent may comprise neuromuscular blocking agents such as suxamethonium, alcuronium, pancuronium, atracurium, gallamine, tubocurarine and vecuronium.

An active agent may comprise sclerosing agents or sclerosants which may be a surfactant, or it may be selected from the group consisting of ethanol, dimethyl sulfoxide, sucrose, sodium chloride, dextrose, glycerin, minocycline, tetracycline, doxycycline, polidocanol, sodium tetradecyl sulfate, sodium morrhuate and sotradecol. An active agent may comprise an angiogenesis inhibitor. An active agent may comprise a 5-lipoxygenase inhibitor or antagonist. An active agent may comprise a chemokine receptor antagonist.

An active agent may comprise a cell cycle inhibitor such as a taxane; an anti-microtubule agent; paclitaxel; an analogue or derivative of paclitaxel; a vinca alkaloid; camptothecin or an analogue or derivative thereof; a podophyllotoxin, wherein the podophyllotoxin may be an etoposide or an analogue or derivative thereof; an anthracycline, wherein the anthracycline may be doxorubicin or an analogue or derivative thereof or the anthracycline may be mitoxantrone or an analogue or derivative thereof; a platinum compound; a nitrosourea; a nitroimidazole; a folic acid antagonist; a cytidine analogue; a pyrimidine analogue; a fluoropyrimidine analogue; a purine analogue; a nitrogen mustard or an analogue or derivative thereof; a hydroxyurea; a mytomicin or an analogue or derivative thereof, for example mitomycin A, mitomycin B, and mitomycin C; an alkyl sulfonate; a benzamide or an analogue or derivative thereof; a nicotinamide or an analogue or derivative thereof; a halogenated sugar or an analogue or derivative thereof; a DNA alkylating agent; an anti-microtubule agent; a topoisomerase inhibitor; a DNA cleaving agent; an antimetabolite; a nucleotide interconversion inhibitor; a hydroorotate dehydrogenase inhibitor; a DNA intercalation agent; an RNA synthesis inhibitor; a pyrimidine synthesis inhibitor; a cyclin dependent protein kinase inhibitor; an epidermal growth factor kinase inhibitor; an elastase inhibitor; a factor Xa inhibitor; a farnesyltransferase inhibitor; a fibrinogen antagonist; a guanylate cyclase stimulant; a heat shock protein 90 antagonist; which may be a geldanamycin or an analogue or derivative thereof; a guanylate cyclase stimulant; a HMGCoA reductase inhibitor, which may be simvastatin or an analogue or derivative thereof; an IKK2 inhibitor; an IL-1 antagonist; an ICE antagonist; an IRAK antagonist; an IL-4 agonist; an immunomodulatory agent; sirolimus or an analogue or derivative thereof; everolimus or an analogue or derivative thereof; tacrolimus or an analogue or derivative thereof; biolmus or an analogue or derivative thereof; tresperimus or an analogue or derivative thereof; auranofin or an analogue or derivative thereof; 27-0-demethylrapamycin or an analogue or derivative thereof; gusperimus or an analogue or derivative thereof; pimecrolimus or an analogue or derivative thereof; ABT-578 or an analogue or derivative thereof; an inosine monophosphate dehydrogenase (IMPDH) inhibitor, which may be mycophenolic acid or an analogue or derivative thereof or 1-α-25 dihydroxy vitamin D₃ or an analogue or derivative thereof; a leukotriene inhibitor; an MCP-1 antagonist; an MMP inhibitor; an NF kappa B inhibitor, which may be Bay 11-7082; an NO antagonist; a p38 MAP kinase inhibitor, which may be SB 202190; a phosphodiesterase inhibitor; a TGF-β inhibitor; a thromboxane A2 antagonist; a TNF-α antagonist; a TACE inhibitor; a tyrosine kinase inhibitor; vitronectin inhibitor; a fibroblast growth factor inhibitor; a protein kinase inhibitor; a PDGF receptor kinase inhibitor; an endothelial growth factor receptor kinase inhibitor; a retinoic acid receptor antagonist; a platelet derived growth factor receptor kinase inhibitor; a fibrinogen antagonist; an antimycotic agent; sulconizole; a bisphosphonate; a phospholipase A1 inhibitor; a histamine H1/H2/H3 receptor antagonist; a macrolide antibiotic; a GPIIb/Illa receptor antagonist; an endothelin receptor antagonist; a peroxisome proliferator-activated receptor agonist; an estrogen receptor agent; a somastostatin analogue; a neurokinin 1 antagonist; a neurokinin 3 antagonist; a VLA-4 antagonist; an osteoclast inhibitor; a DNA topoisomerase ATP hydrolyzing inhibitor; an angiotensin I converting enzyme inhibitor; an angiotensin II antagonist; an enkephalinase inhibitor; a peroxisome proliferator-activated receptor gamma agonist insulin sensitizer; a protein kinase C inhibitor; a ROCK (rho-associated kinase) inhibitor; a CXCR3 inhibitor; Itk inhibitor; a cytosolic phospholipase A₂-α inhibitor; a PPAR agonist; an immunosuppressant; an Erb inhibitor; an apoptosis agonist; a lipocortin agonist; a VCAM-1 antagonist; a collagen antagonist; an α-2 integrin antagonist; a TNF-α inhibitor; a nitric oxide inhibitor; and a cathepsin inhibitor.

An active agent may comprise anti-fibrin and fibrinolytic agents including plasmin, streptokinase, single chain urokinase, urokinase, t-PA (tissue type plasminogen activator) and aminocaproic acid. An active agent may comprise anti-platelet agents including aspirin and prostacyclins (and analogues). An active agent may comprise glycoprotein Ilb/Illa agents including monoclonal antibodies and peptides (e.g. ReoPro, Cilastagel, eptifibatide, tirofiban, ticlopidine, Vapiprost, dipyridamole, forskolin, angiopeptin, argatroban).

An active agent may comprise thromboxane inhibitors; anti-thrombin and anti-coagulant agents, including dextan, heparin, LMW heparin (Enoxaparin, Dalteparin), hirudin, recombinant hirudin, anti-thrombin, synthetic antithrombins, thrombin inhibitors, Warfarin (and other coumarins).

An active agent may comprise anti-mitotic, antiproliferative and cytostatic agents, including vincristine, vinblastine, paclitaxel, methotrexate, cisplatin, fluorouracil, Gemcitabine, rapamycin, azathioprine, cyclophosphamide, mycophenolic acid, corticosteroids, colchicine, nitroprusside; antiangiogenic and angiostatic agents, including paclitaxel, angiostatin and endostatin. An active agent may comprise ACE inhibitors (e.g. Cilazapril, Lisinopril, Captopril).

An active agent may comprise antioxidants, minerals, and vitamins (e.g. Probucol, Tocopherol, Vitamins A, C, B1, B2, B6, B 12, B 12-alpha, and E, vitamin E acetate and vitamin E sorbate, calcium, magnesium, iron, copper, selenium); calcium channel blockers (e.g. nifedipine); fish oil (omega 3-fatty acid); phosphodiesterase inhibitors (e.g. dipyridamole); nitric acid donors (e.g. Molsidomine); somatostatin analogues (e.g., angiopeptin); immunosuppresives and anti-inflammatory agents (e.g. prednisolone, glucocorticoid and dexamethasone); radionuclides such as α, β and γ emitting isotopes (e.g. Re-188, Re-186, I-125, Y-90); COX-2 inhibitors such as Celecoxib and Vioxx; kinase inhibitors such as epidermal growth factor kinase inhibitor, tyrosine kinase inhibitors, MAP kinase inhibitors protein transferase inhibitors, Resten-NG; smoking cessation agents such as nicotine, bupropion and ibogaine; insecticides and other pesticides which are suitable for local application; vitamins A, C, B1, B2, B6, B 12, B 12-alpha, and E, vitamin E acetate and vitamin E sorbate.

An active agent may comprise allergens for desensitization such as house, dust or mite allergens, grasses, trees, pollens, food molecules, sensitizing chemicals, and other known allergens; nutritional agents and nutraceuticals, such as vitamins, essential amino acids and fats; macromolecular pharmacologically active agents such as proteins, enzymes, peptides, polysaccharides (such as cellulose, amylose, dextran, chitin), nucleic acids, cells, tissues, and the like; bone mending biochemicals such as calcium carbonate, calcium phosphate, hydroxyapetite or bone morphogenic protein (BMP); angiogenic growth factors such as Vascular Endothelial Growth Factor (VEGF) and epidermal growth factor (EFG); cytokines interleukins; fibroblasts; cytotaxic chemicals; keratolytics such as the alpha-hydroxy acids, glycolic acid and salicylic acid; DNA, RNA or other oligonucleotides or polynucleotides.

An active agent may comprise vaccines, including vaccines known and used for humans and animals. For example, human-related vaccines, including, but are not limited to, measles, mumps, varicella, polio, pertussis, typhoid, staphylococcus, and those vaccines for oncogenic treatments (e.g., poliovirus for glioblastoma) or genetic transformative vaccines, (e.g., AAV or adenovirus. For example, vaccines for animals include, but are not limited to, Hendra virus (HeV) G glycoprotein and/or Nipah virus G glycoprotein, Lutenising Hormone Releasing Hormone (LHRH) peptide, LHRH-diphtheria toxoid conjugate, porcine circovirus type 2 (PCV2) antigen, a porcine reproductive and respiratory syndrome virus antigen, Mycoplasma hyopneumoniae protein antigen, proteins or protein fragments, for example ORFI Torque teno virus protein, or other TTV proteins or fragments, antigens against Aeromonas salmonicida, antigens against Vibrio anguillarum, and antigens against V. salmonicida.

An active agent may comprise growth factors such as Vascular Endothelial Growth Factor (VEGF) and epidermal growth factor (EFG), Fibroblast Growth Factors (FGF-1 through FGF-23), Interleukins (IL-1 through IL-13), Insulin-like Growth Factor-1, platelet derived growth factor (PDGF), nerve growth factors, neutrophins [Brain-derived neurotrophic factor (BDNF), Nerve growth factor (NGF), Neurotrophin-3 (NT-3), Neurotrophin-4 (NT-4)], Transforming growth factors (TGF-α, TGF-β), Tumor necrosis factor (TNF); and growth factor agonists or antagonists as well as antibodies against these growth factors. An active agent may be an inhibitor of a receptor for the above growth factors.

In an aspect, an active agent is a protein, where that term includes peptides and polypeptides, sugar-modified protein such as glycoprotein, as well as functional descriptions of protein classes such as antigen, enzyme, immunoglobulin and antibody. A composition may include a special delivery vehicle for the active agent, such as a virus or modified virus, where the active agent, such as a protein or polynucleotide, is contained within or expressed by the special delivery vehicle.

Where the protein has a net charge, for example a net positive charge, an absorbable polymer may have a complementary charge, for example an absorbable polymer may have a net negative charge and can bind An active agent with a net positive charge. In this way, the active agent will be attracted by ionic charge interaction to the absorbable polymer, and is thus slowly released from the in situ deposited composition. Alternatively, when faster release of an active agent is desired, an absorbable polymer may have the same net charge as the active agent. For instance, if the active agent has a net negative charge, then the absorbable polymer will also have a net negative charge, and the active agent will be quickly released from the in situ gelled composition.

In an aspect, an active agent is a PD-L1 inhibitor. A PD-L1 inhibitor can include but is not limited to Atezolizumab, Avelumab, Durvalumab, LY3300054 (Eli Lilly and Company), and monoclonal antibodies or monoclonal antibody conjugates that act as a PD-L1 inhibitor

In an aspect, an active agent is a PD-1 inhibitor A PD-1 inhibitor can include but is not limited to pembrolizumab, Nivolumab, Cemiplimab and monoclonal antibodies or monoclonal antibody conjugates that act as a PD-1 inhibitors.

In an aspect, an active agent is a CTLA-4 inhibitor. A CTLA-4 inhibitor can include but is not limited to Ipilimumab, AGEN1884 and monoclonal antibodies or monoclonal antibody conjugates that act as a CTLA-4 inhibitor.

In an aspect, an active agent is a compound that is used to treat non-muscle invasive bladder cancer. The compounds that can be used include but are not limited to non-live immunologically active Bacillus Calmette-Guerin (BCG) subcomponents that include BCG cell wall and various BCG proteins and antigens, an IL-2 fusion protein such as ALT-801 (Altor Bioscience), Oportuzumab monatox (Sesen Bio), sunitinib (Pfizer), enzalutamide, ethacrynic acid, imiquimod and tamoxifen, ALT-803 (Altor Bioscience), and Lenalidomide.

In an aspect, an active agent is an antibody drug conjugate. The antibody drug conjugate can include but is not limited to trastuzumab emtansine, Sacituzumab govitecan, Enfortumab vedotin, ASG-15ME, Gemtuzumab ozogamicin, Brentuximab vedotin, Trastuzumab emtansine, and Inotuzumab ozogamicin.

In an aspect, an active agent is a small molecule protein kinase inhibitor. The small molecule protein kinase inhibitor can include but is not limited to abenaciclib, acalabrutini, afatinib, alectinib, axitinib, baricitinib, binimetinib, bosutinib, brigatinib, cabozantinib, ceritinib, cobimetinib, crisotinib, dabrafenib, dacomitinib, dasatinib, encorafenib, erdafitinib, erlotinib, everolimus, fostamatinib, gefitinib, gilteritinib, ibrutinib, imatinib, lapatinib, larotrectinib, lenvatinib, lorlatinib, midostaurin, neratinib, netarsudil, nilotinib, nintedanib, osimertinib, palbociclib, Pazopanib, Ponatinib, Regorafenib, Ribociclib, Ruxolitinib, Sirolimus, Sorafenib, Sunitinib, Temsirolimus, Tofacitinib, Trametinib, Vandetanib, and Vemurafenib.

In an aspect, a disclosed degradable medical device and/or composition may be manufactured to contain or comprise and release one or more of these or other active agents. In addition to the active agents listed herein, pharmaceutically acceptable salts, esters, and other derivatives of the active agents can also be utilized. The active agents provided herein can be loaded, for example, into a polymeric component of the medical device and/or composition. An active agent may be incorporated into a portion of a degradable medical device and/or composition, for example, the coil, a knitted construct that adjoins the coil, or a coating which impregnates the knitted construct. An active agent may be incorporated into a coating which is coated onto the degradable medical device and/or composition.

Active agents may be may be contained or comprised by a degradable medical device and/or composition in known ways, including the following, among others wherein the active agent contacts an entire component or the entire device, or a portion of a component or a device and is: (a) loaded in the interior (in a composition that fills the interior) of a component, e.g., filling the interior hollow tubing of a monofilament coil, or in the interstices of a multifilament knitted construct, or in a coating or sleeve or sheath, (b) bound to a surface of the medical device and/or composition, such as a surface of a monofilament coil, or a surface of the multifilament knitted construct, or a surface of a containment layer or a sleeve or sheath, where the active agent is bound to the surface by any of covalent interactions and/or non-covalent interactions (e.g., interactions such as van der Waals forces, hydrophobic interactions and/or electrostatic interactions, for instance, charge-charge interactions, charge-dipole interactions, and dipole-dipole interactions, including hydrogen bonding), (c) applied as a coating that covers all or a portion of the device or a component thereof, (d) loaded in surface features (e.g., depressions) of a device or a component thereof, and (e) combinations of the forgoing.

The amount of active agent(s) associated with the active agent-releasing degradable medical device and/or composition is generally a prophylactically effective amount, where that amount may range, for example, from than 1 wt % or less to 2 wt % to 5 wt % to 10 wt % to 25 wt % to 50 wt % or more, depending the particular active agent and the desired effect or treatment regimen.

In an aspect, the present disclosure describes a degradable medical device and/or composition comprising an active agent, generally for placement in or on a body of a mammal, such a device comprising: a polymeric matrix forming the device and defining a lumen through at least a portion of the device, the matrix comprising polymer macromolecules and defining spaces between the polymer macromolecules; an active agent contained within at least some of the spaces of the matrix and/or the lumen; optionally, the active agent is comprised by a composition contained within at least some of the spaces of the matrix to affect diffusion of the active agent out of the polymeric matrix when the medical device and/or composition is placed in the body of the subject. As used herein, the term active agent includes the salt, ester or fragments (e.g. of a protein) of an active agent, and optionally, the active agent is provided in a composition which may comprise salts, pharmaceutical diluents, excipients, or other known stabilizing components, or other compounds that aid in the retention of the active agent in the device before implantation and/or administration of the active agent to the subject. Optionally, one or more of the following may further characterize a degradable medical device and/or composition of the present disclosure: each of the polymeric material and the active agent has a molecular weight where the molecular weight of the active agent is less than the molecular weight of the polymeric material; the quantity of active agent associated with the device is between 0.1 and 50 weight percent of the weight of the device; the medical device and/or composition is an intravesical drug-eluting device; the polymeric component comprises ethylene vinyl acetate (EVA); the polymeric component is hydrophobic; at least some of the spaces that contain the active agent also contain polymeric material; the active agent comprises oxybutynin chloride or ketorolac; the material in association with the active agent comprises polyethylene glycol (PEG); the active agent is in association with a biodegradable material; the active agent is in association with a material from which the active agent must dissociate before diffusing out of the polymeric matrix; the polymeric matrix is coated onto the device.

A disclosed degradable medical device and/or composition can be used as a vehicle to deliver one or more active agents to the body of a patient. A degradable medical device and/or composition can be used to deliver the active agent(s) by placing the device entirely or partially in the body of a subject. By using certain material(s) and active agent(s) in a polymeric matrix, the diffusion of the active agent(s) out of the matrix can be controlled in ways previously unachievable. One or more active agents may thereby be administered to the subject's body over a sustained time (ranging from days to months, for example) and at a relatively constant, and active, level.

An active agent-delivering degradable medical device and/or composition according to the present disclosure may be formed entirely or partially of a polymeric matrix, which comprises with the active agent(s) and material(s) that affect the diffusion of the active agent(s) out of the matrix when the device is placed in the subject. A device according to the present disclosure may optionally be coated entirely or partially with such a loaded polymeric matrix. For example, a hydrophobic polymeric matrix can coat all or some portion of a device.

In an aspect, the present disclosure provides an urinary intravesical active agent-eluting degradable medical device and/or composition comprising an elongated tubular body, a deployable retention structure, and an active agent-releasing member selected from (i) a sleeve of active agent-releasing material that is disposed over at least a portion of the deployable retention structure, (ii) a sheet of active agent-releasing material that is attached to the deployable retention structure and (iii) a sheet of active agent-releasing material connected to a sleeve of material that is disposed over at least a portion of the deployable retention structure. Optionally, one or more of the following features may further describe this active agent-releasing intravesical device: a sleeve of active agent-releasing material is disposed over at least a portion of the deployable retention structure, where optionally the sleeve is a biodegradable sleeve and/or the sleeve is a heat shrinkable sleeve and/or the sleeve ranges from 1 to 4 mm in inner diameter, from 2 to 500 mm in length and from 50 to 200 micrometers in thickness; the intravesical device comprises a sheet of active agent-releasing material that is attached to the deployable retention structure, where optionally, the sheet is a biodegradable sheet and/or the sheet is an elastic sheet and/or the sheet ranges from 2 to 20 mm in width, from 2 to 500 mm in length and from 50 to 200 micrometers in thickness; the intravesical device includes a retention structure in the form of a coil or a loop and wherein the sheet of active agent-releasing material spans a majority of the coil or loop area upon deployment of the retention structure; the intravesical device includes a sheet of active agent-releasing material connected to a sleeve of material that is disposed over at least a portion of the deployable retention structure; the intravesical device includes a retention structure which is a kidney retention structure configured to be delivered through the ureter and deployed in the kidney, where optionally the retention structure is adapted to be reduced to a profile that is sufficiently small during deployment to allow the retention structure to be delivered to the kidney while an active agent is delivered in the bladder by an active agent-releasing member and a tubular section traverses at least one ureter; the intravesical device has a retention structure that comprises a plurality of elongated elements to which the sheet of active agent-releasing material is attached and between which the sheet of active agent-releasing material is situated upon deployment of the retention structure; the intravesical device body and deployable retention structure comprise a biostable polymer.

The amount of the active agent in a polymer matrix may be between about 0 to 50 weight percent of the device depending on the nature of the active agent, the quantity of the polymer, the release profile of the polymer, the release profile of the active agent, the desired active agent diffusion effect, and the desired period for active agent delivery, among other factors. In one aspect, the amount of the active agent is between about 1 to 10 weight percent of the device; is between about 1 to 20 weight percent of the device, is between about 10 to 20 weight percent of the device, oris between about 2 to 50 weight percent of the device.

Compounds or molecules (“materials”) may be added to the polymer composition specifically to influence the release of the active agent from the polymer. Such materials include, without limitation, styrene-block-isobutylene-block-styrene (SIBS), collagen, alginates, carboxymethyl cellulose (CMC), hydroxypropyl cellulose (HPC), dextrin, plasticizers, lipophilic material and other fatty acid salts, pore formers, sugar, glucose, starch, hyaluronic acid (HA), chelating agents, including ethylenediaminetetraacetic acid (EDTA), polyethylene glycol (PEG), polyethylene oxide (PEO), and copolymers thereof. Multiple materials of varying release profiles may be incorporated within the polymeric composition with the active agent(s) to achieve the desired active agent release profile.

In one aspect, the present disclosure provides a degradable medical device and/or composition that is at least partially covered by a containment layer, where the containment layer is either nonbioabsorbable, or is at least partially bioabsorbable but does not degrade as quickly as does the medical device and/or composition. In one aspect, in vivo, the medical device and/or composition will degrade into pieces (fragments of the original device) while the containment layer retains sufficient structural integrity to provide a barrier which cannot be crossed by the pieces from the medical device and/or composition. In this way, the pieces are constrained to staying in a localized area where they cannot cause any harm to the subject. In fact, even as the pieces degrade, the resulting smaller pieces and ultimately the molecular components of the medical device and/or composition, will all stay within the outer containment layer, and together may be conducted to a place that is safe for elimination.

Optionally, the containment layer and the degraded medical device and/or composition are simultaneously eliminated. Both the degradable medical device and/or composition and the containment layer may become soft and compliant, and may travel along a tube into which they were implanted, e.g., the ureter or urethra, until they are simultaneously eliminated from the subject. The containment layer may be degradable, however it need not be degradable. So long as the containment layer becomes soft and flexible, and maintains its integrity, it may be eliminated from the subject at the same time that the medical device and/or composition is eliminated.

The containment layer may assist in managing the segmentation of the device, i.e., the disintegration of the device into pieces. The containment layer may, for example, be located over regions of the device and protect those regions from contact with the subject's fluids. The unprotected regions will degrade more quickly, and may lead to breaks or segments in the medical device and/or composition. The containment layer may be porous, and so allow a controlled amount of the subject's surrounding fluid to contact the medical device and/or composition. By adjusting the porosity of the containment layer, the segmentation of the device may be managed. As another option, by using a gradient coating, the parts of the medical device and/or composition covered by the relatively thinner (less coating) regions may break into segments first. As another option, the medical device and/or composition may be pre-degraded by, e.g., exposing selected portions of the device to moisture for some period of time. These pre-selected regions may prematurely degrade, relative to other parts of the medical device and/or composition. In this case, the containment layer may be located over the pre-degraded regions, to control when those regions are exposed to the subject's fluids.

While the containment layer may be present on the outside of the degradable medical device and/or composition, an alternative aspect positions the containment layer on the inside of a medical device and/or composition, for example, on the inside of a medical device and/or composition which has a hollow space, i.e., a lumen. When a containment layer is positioned on the inside of a device, and the device is biodegradable, then the pieces of device that form during degradation may held, for example between inner and outer containment layers, or a containment layer and the tissue of the subject, for a sufficient time so that the pieces become manageably small, that is, they are not of a size that is harmful to the subject, or alternatively the pieces degrade into their component polymeric or monomeric components and can migrate through the containment layer.

In an aspect, a degradable medical device and/or composition is characterized as having a gradient. A gradient refers to variation in some property, e.g., composition, of the medical device and/or composition as a function of a direction. This gradient provides for variation in degradation along the gradient. For example, the average molecular weight of the polymer that forms a medical device and/or composition may vary along a direction of the medical device and/or composition, such that the polymer at the distal end of the medical device and/or composition or a portion thereof has a higher average molecular weight than does the polymer at a proximal end of the degradable medical device and/or composition or a portion thereof. In this way, the proximal end of the degradable medical device and/or composition or portion thereof may degrade more rapidly than does the distal end where the polymer has a higher initial average molecular weight. The provision of a gradient in a degradable medical devices and/or compositions of the present disclosure provides a mechanism for managed degradation of the device. In one aspect, the gradient does not impact or effect the functionality of the medical device and/or composition, but only impacts the degradation profile of the device. Such nonhomogeneity in a degradable medical device and/or composition may be referred to herein as the gradient of the degradable medical device and/or composition, and a degradable medical device and/or composition having such a gradient may be referred to as a graduated degradable medical device and/or composition.

Optionally, a containment layer of the present disclosure may be characterized in terms of having a gradient, such that the containment layer that covers one portion of a degradable medical device and/or composition is different from the containment layer that covers another portion of the degradable medical device and/or composition. Such nonhomogeneity in the containment layer will be referred to herein as the gradient in the containment layer, and the containment layer having such a gradient may be referred to as a graduated containment layer.

For example, in one aspect gradients provide different degradation rates. Gradients may be constructed such that the containment layer covering one portion of a medical device and/or composition degrades at a different rate, either faster or slower, than does the containment layer covering a different portion of the medical device and/or composition.

As another example, in another aspect gradients provide different degrees of degradation. Thus, gradients may be constructed such that the containment layer covering one portion of a degradable medical device and/or composition will degrade to a different extent than does the containment layer covering a different portion of the degradable medical device and/or composition. The extent of degradation may be measured in different ways. For instance, the thickness of the containment layer may be measured before implantation and then after it has been implanted and degraded to the fullest extent that it will degrade. The change in thickness may be described as a percentage reduction in thickness, where the gradient provides for a different percentage reduction in thickness over one portion of the degradable medical device and/or composition, either greater or less, compared to the reduction in thickness that occurs over a different portion of the degradable medical device and/or composition.

As yet another example, in one aspect the gradient provides different sized holes in the containment layer at different locations, or optionally holes in one location but no holes in another location of the containment layer. In other words, the containment layer may have variation in porosity. Thus, the gradients may be constructed such that the containment layer over one portion of a degradable medical device and/or composition is in the form of a mesh, net, weave or other construct that includes holes, while the containment layer over a different portion of the degradable medical device and/or composition is solid, i.e., does not have any holes. Alternatively, the gradients may be constructed such that containment layer over one portion of the medical device and/or composition is in the form of a mesh etc. with relatively large holes, while the containment layer over a different portion of the medical device and/or composition is also l the form of a mesh etc. but with relatively smaller holes.

Gradients may be formed in various ways. For example, different compositions, having different degradation rates, may be used to form the containment layer over different portions of the medical device and/or composition. Thus, a composition having a relatively high degradation rate may be placed over a first portion of a degradable medical device and/or composition while a composition having a relatively slower degradation rate maybe placed over a second portion of the degradable medical device and/or composition. In this way, the containment layer will degrade more quickly in some places than in other places.

As another example, a single composition may be used to form a graduated containment layer. For instance, a single composition may be coated to a first thickness over a first portion of a degradable medical device and/or composition while the same composition is used to create a coating having a second thickness over a second portion of the degradable medical device and/or composition. In general, a thicker coating will be retained for a longer time on a degradable medical device and/or composition than will a thinner coating, or in other words, a thicker coating will degrade more slowly than a thinner coating, all other factors being equal. A thicker coating may be formed, for example, by repeatedly coating a region of the containment layer where greater coating thickness is desired.

The thickness of the containment layer may vary throughout a degradable medical device and/or composition. However, at its thickest point, in various aspects, the containment layer has a thickness of greater than 10 microns, or greater than 20 microns, or greater than 30 microns, or greater than 40 microns, or greater than 50 microns, or greater than 60 microns, or greater than 70 microns, or greater than 80 microns, or greater than 90 microns, or greater than 100 microns, or greater than 110 microns, or greater than 120 microns, or greater than 130 microns, or greater than 140 microns, or greater than 150 microns, or greater than 160 microns, or greater than 170 microns, or greater than 180 microns, or greater than 190 microns, or greater than 200 microns. The maximum thickness may be 500 microns, or 400 microns, or 300 microns, or 200 microns, or 150 microns, or 100 microns. As mentioned previously, the containment layer may be a coating, where the thickness of the coating at its thickest part is any of the aforementioned thicknesses.

The amount of the containment layer may vary throughout a degradable medical device and/or composition. In one aspect, in addition to or instead of specifying a thickness for a containment layer, a containment layer may be characterized in terms of how much organic polymer is present over a given volume of medical device and/or composition. For example, the amount may be specified in terms of mg organic polymer per square centimeter (cm²) of medical device and/or composition. In various aspects, the medical device and/or composition is covered with containment layer in the amount of at least 10 mg/cm²; or at least 15 mg/cm²; or at least 20 mg/cm²; or at least 25 mg/cm²; or at least 30 mg/cm²; or at least 35 mg/cm²; or at least 40 mg/cm²; or at least 45 mg/cm²; or at least 50 mg/cm².

As yet another example, the filaments that form a weave may be woven tighter or looser in order to affect the number and size of the holes in the weave. A containment layer may be constructed from two or more different weaves, providing larger holes over a first portion of a medical device and/or composition and smaller holes over a second portion of the medical device and/or composition. In this way, the containment layer will allow the underlying medical device and/or composition to degrade more quickly in the first portion of the medical device and/or composition (where the mesh hole sizes are larger and so the mesh affords more access of the surrounding body fluids to the medical device) and more slowly in the second portion of the medical device and/or composition (where the mesh hole sizes are smaller).

Thus, in one aspect the present disclosure provides a degradable medical device and/or composition comprising a medical device and/or composition and a graduated containment layer that covers at least a portion of the medical device and/or composition. Optionally, the graduated containment layer may comprise multiple thicknesses, e.g., 2, 3, 4, 5, or more than 5 different thicknesses at different locations. The graduated containment layer having multiple thicknesses at different locations may be formed by having various numbers of coating layers of a polymer composition at different locations, and thus may be said to comprise multiple layers (of coating composition). Also optionally, the graduated containment layer may comprise multiple compositions, e.g., 2, 3, 4, 5, or more than 5 different compositions at different locations. Optionally, the graduated containment layer may comprise variation in two or more properties, e.g., multiple thicknesses and multiple compositions.

While thickness, composition and porosity are examples of variation that may be present in a containment layer, these are exemplary only. Other variations can also be used to create a graduated containment layer according to the present disclosure, for example, variation in texture, variation in hydrophilicity, variation in thermal stability, variation in tensile strength, and variation in fiber density when the containment layer contains fibers, to name a few.

In one aspect, a containment layer is made from one or more organic polymers. A containment layer may be completely non-biodegradable. However, in another aspect, a containment layer is biodegradable, but it degrades at a slower rate than the medical device and/or composition. In this way, if a degradable medical device and/or composition is degrading into pieces, the containment layer retains its structural integrity and holds the pieces together within a confined space, for a time sufficient for the pieces to degrade into even smaller pieces that are not harmful to the subject, and/or into the polymeric and/or monomeric components of the medical device and/or composition.

In one aspect, a containment layer is a coating on a degradable medical device and/or composition. The coating may be present on all of the exposed surfaces of the degradable medical device and/or composition, or only one some of those surfaces, e.g., the sides. The coating may be completely non-biodegradable. However, in another aspect, the coating is biodegradable, but it degrades at a slower rate than the degradable medical device and/or composition. In this way, if the degradable medical device and/or composition is degrading into pieces, the coating retains its structural integrity and holds the pieces together within a confined space, for a time sufficient for the pieces to degrade into even smaller pieces that are not harmful to the subject, and/or into the polymeric and/or monomeric components of the medical device and/or composition.

Particularly when a coating is biodegradable, and a degradable medical device and/or composition is disintegrating into pieces, the coating can maintain sufficient strength during the period of time when the medical device and/or composition disintegrates, such that the coating will be able to contain the pieces within the coating. To provide this function, the coating must be of adequate thickness. In order to provide a coating of adequate thickness, the medical device and/or composition may be dipped into a polymer solution, i.e., a solution of dissolved polymer. The device may be dipped multiple times into the solution, in order to build up a thickness of polymer that will maintain sufficient strength and integrity to function as a containment layer during the disintegration of the medical device and/or composition. Alternatively, a degradable medical device and/or composition may be drawn through a polymer solution. The rate at which the device is drawn through the solution will impact the thickness of the coating: a slower draw rate will provide for a thicker coating.

When a polymer solution is used to form the coating on a degradable medical device and/or composition, the concentration of polymer in the solution is also a factor that must be considered. A higher concentration of polymer will tend to deposit more polymer on the surface of the medical device and/or composition, when that device is dipped, drawn, or otherwise coated with the polymer so as to form a containment layer.

A containment layer is placed on those portions of a degradable medical device and/or composition where it is desired to protect the subject from damage or injury or trauma due to pieces of the device being formed during biodegradation.

Optionally, a degradable medical device and/or composition may not break into smaller pieces, but may instead soften to such a degree that it may pass through the conduit in which it was implanted.

In one aspect, the present disclosure provides intravesical degradable medical devices and/or compositions which have a diversity of properties at different locations of the intravesical device, but the intravesical device and components thereof are not assembled from multiple segments. Rather, the intravesical degradable medical device and/or composition is assembled from a single uniform construct, and that construct is then modified to provide a diversity of properties at different locations of the construct. The diversity may in one or more properties including biodegradability, radiopacity, stiffness or flexibility, and loading with active agents. The diversity is created by methods as disclosed herein, e.g., by cutting a slit in a component of the intravesical device, by selectively degrading the intravesical device or a component thereof before it is implanted into the subject, and other methods disclosed herein.

In one aspect, a degradable medical device and/or composition is a intravesical device, and the intravesical device is a construct of a fiber-reinforced elastomeric film designed with at least one position-retaining end, wherein the fiber-reinforcement is (a) a combination of a monofilament coil and weft-knitted tube multifilament yarn; (b) a combination of monofilament coil and a braided multifilament yarn; (c) a tube comprising a braided or weft-knitted monofilament yarn; or (d) a weft-knitted or braided monofilament yarn in the form of a tube.

In still another aspect, an intravesical degradable medical device and/or composition is a construct of a fiber-reinforced elastomeric film designed with at least one position-retaining end wherein the fiber reinforcement is a combination of a monofilament and knitted or braided multifilament yarn, wherein the fiber reinforced elastomeric film is in the form of a tube with a central, main component having a smaller diameter than that of the patient ureter wherein each of the position-retaining ends defines two freely laterally deformable components formed of initially partially overlapping bi-tubular ends of the main, central component and a laterally fused tube which are radially and axially cut to produce two over-extended flaps attached to an intact semi-cylindrical extension of the main, central tube.

In yet still another aspect, an intravesical degradable medical device and/or composition is a construct of a fiber-reinforced elastomeric film designed with at least one position-retaining end, wherein the fiber-reinforcement is a monofilament yarn or a combination with knitted or braided multifilament yarn, wherein the fiber-reinforced elastomeric film is in the form of a tube with a smaller diameter than that of the patient ureter and having at least one position-retaining end, wherein the position-retaining end is an angled portion of the main tube having a length comparable to the patient ureter and comprising a flexible hinge that maintains an angle of more than 30 degrees with respect to the main tube in an absence of deforming stress.

In another aspect, an intravesical degradable medical device and/or composition comprises a retention portion configured to help retain the intravesical device in place within a body of a patient; and an elongate portion extending from the retention portion, the elongate portion having a sidewall defining a lumen, the sidewall having a first section and second section, the first section of the sidewall having a first thickness, the second section of the sidewall having a second thickness different than the first thickness. Optionally, the intravesical device may be further characterized by one or more of the following: the retention portion is configured to be disposed within a kidney of the patient; the retention portion is a first retention potion and the intravesical device further comprises a second retention portion configured to help retain the intravesical device in place within the body of the patient; the first section of the sidewall forms an annular ring; the first section of the sidewall forms a spiral; the first section of the sidewall forms a dimple; the sidewall has a third portion, the second portion of the sidewall being disposed between the first portion of the sidewall and the third portion of the sidewall; the sidewall has a third portion, the third portion has a thickness different than the second thickness, the second portion of the sidewall being disposed between the first portion of the sidewall and the third portion of the sidewall; the sidewall has a third portion, the second portion of the sidewall being disposed between the first portion of the sidewall and the third portion of the sidewall, the third portion having a third thickness, the second thickness being greater than the first thickness, the second thickness being greater than the third thickness; the first portion of the sidewall has a first section and a second section, the first section of the first portion forming a spiral rotating in a first direction, the second section of the first portion forming a spiral rotating in a second direction different than the first direction. This intravesical device, including optional aspects thereof, may be modified by techniques disclosed herein to display managed degradation when the intravesical device is located within a subject. For instance, a slit may be made in the slit to provide a site that promotes degradation.

In another aspect an intravesical degradable medical device and/or composition comprises a retention portion configured to help retain the intravesical device in place within a body of a subject; and an elongate portion extending from the retention portion, the elongate portion having a first member and a second member, the first member being devoid of a lumen, the second member being devoid of a lumen, the first member and the second member being intertwined. In another aspect, the intravesical device comprises a retention portion configured to help retain the intravesical device in place within a body of a patient; and an elongate portion extending from the retention portion and having an expanded configuration and a nominal configuration, the elongate portion having a sidewall defining a lumen extending from a first end portion of the elongate portion to a second end portion of the elongate portion, the sidewall defining a chamber, the chamber being configured to receive a fluid to place the elongate portion in its expanded configuration. Again, either of these intravesical devices may be modified by techniques disclosed herein to demonstrate managed degradation with situated within a subject.

In another aspect, an intravesical degradable medical device and/or composition is a construct of a fiber-reinforced elastomeric film designed with at least one position-retaining end wherein the fiber reinforcement is a combination of a monofilament and knitted or braided multifilament yarn, wherein the fiber-reinforced film is tubular with a central main component having a smaller diameter than that of the patient ureter and comprising at least one position-retaining end wherein the position-retaining end is a highly flexible extension of the central main tube, acquiring a goose-neck shape after insertion in the patient ureter but can be made co-linear with the central main tube during insertion with an applicator.

In another aspect, an intravesical degradable medical device and/or composition comprises an elongate member having a first portion and a second portion, the second portion having a sidewall that defines a single lumen, the first portion being coupled to the second portion, the first portion configured to be disposed within a kidney of a patient, the sidewall of the second portion of the elongate member is configured to deliver fluid from a first location of the sidewall of the second portion to a second location of the sidewall of the second portion via at least one of capillary action and wicking, the second portion of the elongate member configured to be disposed within at least one of a bladder of a patient and a ureter of the patient, at least a portion of the first portion being disposed within the lumen. Optionally, one or more of the following features may further characterize this intravesical device: the second portion of the elongate member is constructed of a multi-stranded material; the second portion of the elongate member is constructed of a yarn; the second portion of the elongate member has a configuration selected from a group consisting of a braided tube configuration and a long woven strip configuration; the second portion of the elongate member is constructed of a melt spun polypropylene with a high loading of barium sulfate; the intravesical degradable medical device and/or composition further comprises a proximal retention structure configured to be disposed within the bladder of a patient, the proximal retention structure being coupled to the second portion of the elongate member; the intravesical device further comprises a distal retention structure configured to be disposed within the kidney of the patent, the distal retention structure being coupled to the first portion of the elongate member; the first portion is coupled to the second portion via an interference fit; the second portion of the elongate member has a substantially solid tubular shape; the second portion of the elongate member is substantially flexible; the first portion of the elongate member is substantially rigid; the second portion of the elongate member is more flexible than the first portion of the elongate member. This intravesical device, including optional aspects thereof, may be modified to demonstrate managed degradation according to the present disclosure.

In another aspect, a degradable medical device and/or composition is a ureteral intravesical device comprising: an elongate member having a first portion and a second portion, the second portion having a substantially solid cylindrical shape, the first portion being coupled to the second portion, the first portion configured to be disposed within a kidney of a patient, the first portion having a length such that the first portion terminates in at least one of the kidney and ureter of the patient, the second portion of the elongate member configured to deliver fluid from a first location of the second portion to a second location of the second portion via at least one of capillary action and wicking, the second portion of the elongate member configured to be disposed within at least one of a bladder of a patient and the ureter of the patient. This intravesical degradable medical device and/or composition may be modified to demonstrate managed degradation according to the present disclosure.

In another aspect, the intravesical degradable medical device and/or composition contains at least one filament, where this filament has a longitudinal axis and is formed from materials including a bioabsorbable polymeric material. Polymer molecules within the bioabsorbable polymeric material may have a helical orientation which is aligned with respect to the longitudinal axis of the filament. The intravesical device is at least partially bioabsorbed by a patient after insertion of the intravesical device into the patient. For example, the intravesical device may comprise: a braided or woven configuration; a flared end portion at one of a proximal end or a distal end of the intravesical device; and at least one filament having a longitudinal axis and comprising an oriented bioabsorbable polymeric material, wherein polymer molecules within the bioabsorbable polymeric material have a helical orientation which is aligned with respect to the longitudinal axis of the at least one filament. Optionally, one or more of the following may further describe the intravesical device: the proximal end and the distal end comprise the flared end portion; the at least one filament is helically wound along at least a portion of a length of the intravesical device; the intravesical device comprises a plurality of the filaments, where optionally the plurality of the filaments are helically wound along at least a portion of a length of the intravesical device, and where further optionally a first portion of the plurality of the filaments are helically wound in a first direction and a second portion of the plurality of the filaments are helically wound in an opposite direction to the first direction; the plurality of the filaments are braided and helically wound along at least a portion of the length of the intravesical device; the intravesical device comprises filaments of stainless steel or nitinol; the intravesical degradable medical device and/or composition comprises between 12 and 36 helical filaments; where optionally between 6 and 18 filaments are in the form of helices, and are axially displaced in relation to each and wherein the helices extend in a first direction, and wherein an equal number of filaments comprise helices that extend a second direction that is opposite the first direction, the filaments are uniformly arranged about a longitudinal axis of the intravesical device; the oriented bioabsorbable polymeric material comprises a single bioabsorbable polymer or a blend of bioabsorbable polymers; the oriented bioabsorbable polymeric material comprises a polymer selected from poly(α-hydroxy acid) homopolymers, poly(α-hydroxy acid) copolymers and blends thereof; the oriented bioabsorbable polymeric material comprises a polymer selected from polyglycolide, poly-L-lactide, poly-D-lactide, poly-DL-lactide, and blends thereof; the oriented bioabsorbable polymeric material has a crystallinity ranging from 0.1 to 20%; at least one filament comprises a core of the oriented bioabsorbable polymeric material; at least one filament comprises a coating of the oriented bioabsorbable polymeric material; the intravesical device comprises a plurality of oriented filaments that are arranged to form a pattern of geometric diamond-shaped cells; a plurality of filaments are wrapped about one another to form interlocking joints; at least one filament comprises a active agent; and the intravesical degradable medical device and/or composition is selected from a coronary vascular intravesical degradable medical device and/or composition, a peripheral vascular intravesical degradable medical device and/or composition, a urethral intravesical degradable medical device and/or composition, a ureteral intravesical degradable medical device and/or composition, a biliary intravesical degradable medical device and/or composition, a tracheal intravesical degradable medical device and/or composition, a gastrointestinal intravesical degradable medical device and/or composition and an esophageal intravesical degradable medical device and/or composition.

Another optional aspect provides an intravesical degradable medical device and/or composition which is a construct of a fiber-reinforced elastomeric film designed with at least one position-retaining end, wherein the fiber-reinforcement is a monofilament yarn or a combination with knitted or braided multifilament yarn, wherein the fiber-reinforced elastomeric film is in the form of a tube having at least one position-retaining end, wherein the retaining end is an inverted cone having a diameter at the wider cross-section exceeding that of the main tube and that can be reversibly compressed to conform with the main tube diameter, which is also smaller than that of the patient ureter, upon applying radial compressive force in an applicator. It is preferred that the inverted cone is partially slit, yielding a cone wall having at least two leaflets and preferably three to five leaflets to facilitate the radial compression upon insertion with an applicator.

Yet another optional aspect provides a construct of a fiber-reinforced elastomeric degradable medical device and/or composition which is a film having at least one position-retaining end wherein the fiber reinforcement is a combination of a monofilament and knitted or braided multifilament yarn, wherein the elastomeric film is tubular with a central main component having a smaller diameter than that of the patient ureter and with at least one position-retaining end wherein the position-retaining end is an asymmetrically inverted cone with a teardrop cross-section, slit axially, at the peak of the teardrop which has an average diameter at the wider cross-section exceeding that of the central main tube wherein the slit asymmetric cone can be reversibly compressed to conform with the central main tube diameter upon applying radial compressive force in an applicator.

In yet another optional aspect, an intravesical degradable medical device and/or composition is a construct of a fiber-reinforced elastomeric film, wherein the fiber-reinforcement is a monofilament yarn or a combination with knitted or braided multifilament yarn, wherein the reinforced elastomeric film is tubular with a central main component that is a unilaterally, longitudinally crimped, inflatable tube having a circular cross-section that is smaller than that of the patient ureter when outwardly expanded, and having at least one position-retaining end wherein the position-retaining end is a unilaterally crimped, inflatable, asymmetric, inverted cone having a teardrop cross-sectional geometry and a crimp at the peak of the teardrop that is collinear with the crimp of the central main tube, wherein the average diameter of the inverted cone, when outwardly expanded, exceeds that of the central main tube.

Optionally, a fiber-reinforced elastomeric film degradable medical device and/or composition is formed of a segmented copolymer made from a polyethylene glycol and at least one cyclic monomer selected from the group represented by l-lactide, epsilon-caprolactone, trimethylene carbonate, glycolide, a morpholine-dione, p-dioxanone, and 1,5-dioxapan-2-one. Optionally, the film is formed from a mixture of epsilon-caprolactone and glycolide. Optionally, the film is formed from a mixture of L-lactide and glycolide. An exemplary composition of an elastomeric swellable film composition is a crystalline copolymer of a high molecular weight (20-35 kDa) polyethylene glycol (PEG) and 95/5 (molar) mixture of epsilon-caprolactone/glycolide, wherein the weight percent of the PEG component in the copolymer is about 10 percent.

Another exemplary composition of an elastomeric film degradable medical device and/or composition is a crystalline segmented copolymer made in two steps. The first step entails the formation of an amorphous or low melting copolymer made from epsilon-caprolactone, trimethylene carbonate and glycolide by polymerization in the presence of triethanolamine and stannous octanoate as the initiator and catalyst, respectively. In the second step, the product of the first step is reacted with a mixture of l-lactide and epsilon-caprolactone to produce a crystalline triaxial final copolymer.

Optionally, a degradable medical device and/or composition film may be prepared from electrospun fibers. Also optionally, a fiber-reinforced degradable medical device and/or composition film may comprise or contain a monofilament yarn, optionally in combination with knitted or braided multifilament yarn, wherein the reinforcing monofilament yarn is formed of a segmented copolymer made from at least two cyclic monomers selected from the group represented by l-lactide, epsilon-caprolactone, trimethylene carbonate, glycolide, a morpholine-dione, p-dioxanone, and 1,5-dioxapan-2-one. Optionally, it is formed from l-lactide, epsilon-caprolactone, and trimethylene carbonate which is a relatively slowly degrading composition. Optionally, it is formed from glycolide, epsilon-caprolactone, and trimethylene carbonate which is a relatively quickly degrading composition.

The reinforcing monofilament yarn may also be a composite of an inorganic microparticulate dispersed phase of at least one material selected from the group of barium sulfate, zirconium oxide, and absorbable phosphate glass and an absorbable polymeric matrix of a crystalline segmented copolymer made from at least two cyclic monomers selected from the group consisting of l-lactide, epsilon-caprolactone, trimethylene carbonate, glycolide, p-dioxanone, 1,5-dioxepan-2-one, and a morpholine-2,5-dione. Furthermore, the reinforcing monofilament yarn can be a composite of an inorganic microparticulate dispersed phase of at least one material selected from the group consisting of barium sulfate, zirconium oxide, and absorbable phosphate glass and an absorbable polymeric matrix of a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione.

In still yet another aspect, the present disclosure provides a bioabsorbable and disintegratable, multicomponent, non-migrating endoureteral intravesical degradable medical device and/or composition which is a construct of a fiber-reinforced elastomeric film designed with at least one position-retaining end, wherein the fiber-reinforcement is a monofilament yarn or a combination with knitted multifilament or braided yarn, wherein the reinforcing knitted or braided multifilament fabric is formed of a crystalline segmented copolymer. An exemplary composition of such copolymer is a triaxial copolymer made in two steps. The first step entails the formation of an amorphous or low melting triaxial prepolymer using epsilon-caprolactone and/or trimethylene carbonate in the presence of trimethylolpropane and stannous octanoate as the initiator and catalyst, respectively. In the second step, the product of the first step is reacted with glycolide or a mixture of glycolide with epsilon-caprolactone and/or trimethylene carbonate. Another exemplary composition is a copolymer for use in producing knitted or braided multifilament yarn, which is a crystalline copolymer made from a polyethylene glycol and at least one cyclic monomer selected from the group represented by l-lactide, ε-caprolactone; trimethylene carbonate, glycolide, a morpholine-dione, p-dioxanone, and 1,5-dioxapan-2-one, but preferably from a polyethylene glycol, l-lactide, and trimethylene carbonate, and more preferably from a segmented copolymer of l-lactide and trimethylene carbonate. Optionally, the copolymer is made from glycolide and trimethylene carbonate, which provides a relative fast degradation profile for the yarn.

Thus, in one aspect the present disclosure provides an absorbable and disintegratable, multicomponent, non-migrating endoureteral intravesical degradable medical device and/or composition which is a construct of a fiber-reinforced elastomeric film designed with at least one position-retaining end, wherein the fiber-reinforcement is a combination of a monofilament coil and a braided multifilament yarn, and wherein the film is formed of a crystalline segmented copolymer made from a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione. The film may also be formed from a crystalline segmented copolymer made from l-lactide and at least one cyclic monomer selected from the group consisting of glycolide, ε-caprolactone, trimethylene carbonate, p-dioxanone and 1,5-dioxepan-2-one, and a morpholine-2,5-dione.

The present disclosure provides an absorbable and disintegratable multicomponent, non-migrating endoureteral intravesical degradable medical device and/or composition which is a construct of a fiber-reinforced elastomeric film designed with at least one position-retaining end, wherein the fiber-reinforcement is a combination of a monofilament coil and a braided multifilament yarn, and wherein the reinforcing monofilament yarn is formed of a crystalline segmented copolymer made from at least two cyclic monomers selected from the group consisting of l-lactide, ε-caprolactone, trimethylene carbonate, glycolide, a morpholine-2,5-dione, p-dioxanone and 1,5-dioxepan-2-one. Alternatively, the reinforcing monofilament yarn is a composite of an inorganic microparticulate dispersed phase of at least one material selected from the group of barium sulfate, zirconium oxide, and absorbable phosphate glass and an absorbable polymeric matrix of a crystalline segmented copolymer made from at least two cyclic monomers selected from the group consisting of l-lactide, ε-caprolactone, trimethylene carbonate, glycolide, p-dioxanone, 1,5-dioxepan-2-one, and a morpholine-2,5-dione. The reinforcing monofilament yarn can also be a composite of an inorganic microparticulate dispersed phase of at least one material selected from the group of barium sulfate, zirconium oxide, and absorbable phosphate glass and an absorbable polymeric matrix of a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione.

Thus the present disclosure provides an absorbable and disintegratable multicomponent, non-migrating endoureteral intravesical degradable medical device and/or composition which is a construct of a fiber-reinforced elastomeric film designed with at least one position-retaining end, wherein the fiber-reinforcement is a combination of a monofilament coil and a braided multifilament yarn, and wherein the reinforcing braided multifilament fabric is formed of a crystalline segmented copolymer made from a polyethylene glycol and at least one cyclic monomer selected from the group consisting of l-lactide, trimethylene carbonate, ε-caprolactone, glycolide, p-dioxanone, a morpholine-2,5-dione and 1,5 dioxepan-2-one. Alternatively, the reinforcing braided multifilament tube is formed from a crystalline segmented copolymer of l-lactide and at least one cyclic monomer selected from the group consisting of glycolide, ε-caprolactone, trimethylene carbonate, p-dioxanone, 1,5-dioxepan-2-one, and a morpholine-2,5-dione.

In another aspect, the present disclosure provides that an intravesical degradable medical device and/or composition is a construct of a fiber-reinforced elastomeric film designed with at least one position-retaining end, wherein the fiber-reinforcement is a tube of a braided or weft-knitted monofilament yarn, and wherein the fiber-reinforced film is tubular with a central main component having a smaller diameter than that of the patient ureter and having at least one position-retaining end, and wherein the position-retaining end is a highly flexible extension of the central main tube, acquiring a loop shape with an open end parallel to the axis of the central main tube after insertion in the patient ureter and the loop can be made co-linear with the central main tube during insertion with an applicator. The film component of the assembled intravesical device is formed of a crystalline segmented copolymer made from a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione. Alternatively, the film is formed of a crystalline segmented copolymer made from l-lactide and at least one cyclic monomer selected from the group consisting of glycolide, ε-caprolactone, trimethylene carbonate, p-dioxanone and 1,5-dioxepan-2-one, and a morpholine-2,5-dione.

In another aspect, the present disclosure provides that an intravesical degradable medical device and/or composition is a construct of a fiber-reinforced elastomeric film designed with at least one position-retaining end, wherein the fiber-reinforcement is a tube of a braided or weft-knitted monofilament yarn, and wherein the reinforcing braided or weft-knitted monofilament yarn is formed of a crystalline segmented copolymer made from at least two cyclic monomers selected from the group consisting of l-lactide, ε-caprolactone, trimethylene carbonate, glycolide, a morpholine-2,5-dione, p-dioxanone and 1,5-dioxepan-2-one. Alternatively, the reinforcing braided or weft-knitted monofilament yarn is formed of a crystalline segmented copolymer made from a polyethylene glycol and at least one cyclic monomer selected from the group consisting of l-lactide, trimethylene carbonate, ε-caprolactone, glycolide, p-dioxanone, a morpholine-2,5-dione and 1,5 dioxepan-2-one. The reinforcing weft-knitted or braided monofilament can also be a composite of an inorganic microparticulate dispersed phase of at least one material selected from the group of barium sulfate, zirconium oxide, and absorbable phosphate glass and an absorbable polymeric matrix of a crystalline segmented copolymer made from at least two cyclic monomers selected from the group consisting of l-lactide, ε-caprolactone, trimethylene carbonate, glycolide, p-dioxanone, 1,5-dioxepan-2-one, and a morpholine-2,5-dione. Furthermore, the reinforcing weft-knitted or braided monofilament can be a composite of an inorganic microparticulate dispersed phase of at least one material selected from the group of barium sulfate, zirconium oxide, and absorbable phosphate glass and an absorbable polymeric matrix of a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione.

In an aspect, an intravesical degradable medical device and/or composition is a construct of a fiber-reinforced elastomeric film designed with at least one position-retaining end, wherein the fiber reinforcement is a weft-knitted or braided monofilament scaffold and the reinforced construct therefrom is in the form of a tube comprising a central main component having a diameter smaller than that of the patient ureter and at least one position-retaining end, wherein the position-retaining end is an inverted cone having a series of diameters designed to provide progressively wider cross-sections than that of the central main tube and can be reversibly compressed to conform radially with the central main tube upon applying radial compressive force during insertion to the urogenital tract using a tubular applicator, and wherein the film is formed of a crystalline segmented copolymer made from a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione. Alternatively, the film is formed of a crystalline segmented copolymer made from l-lactide and at least one cyclic monomer selected from the group consisting of glycolide, ε-caprolactone, trimethylene carbonate, p-dioxanone and 1,5-dioxepan-2-one, and a morpholine-2,5-dione. The reinforcing weft-knitted or braided monofilament yarn may optionally be formed of a crystalline segmented copolymer made from at least two cyclic monomers selected from the group consisting of l-lactide, ε-caprolactone, trimethylene carbonate, glycolide, a morpholine-2,5-dione, p-dioxanone and 1,5-dioxepan-2-one. Alternatively, the reinforcing braided or weft-knitted monofilament yarn is formed of a crystalline segmented copolymer made from a polyethylene glycol and at least one cyclic monomer selected from the group consisting of l-lactide, trimethylene carbonate, ε-caprolactone, glycolide, p-dioxanone, a morpholine-2,5-dione and 1,5 dioxepan-2-one.

In an aspect, an intravesical degradable medical device and/or composition is a construct of a fiber-reinforced elastomeric film designed with at least one position-retaining end, wherein the fiber reinforcement is a weft-knitted or braided monofilament scaffold and the reinforced construct therefrom is in the form of a tube comprising a central main component having a diameter smaller than that of the patient ureter and at least one position-retaining end, wherein the position-retaining end is an inverted cone having a series of diameters designed to provide progressively wider cross-sections than that of the central main tube and can be reversibly compressed to conform radially with the central main tube upon applying radial compressive force during insertion to the urinogenital tract using a tubular applicator, and wherein the reinforcing weft-knitted or braided monofilament yarn is a composite of an inorganic microparticulate dispersed phase of at least one material selected from the group of barium sulfate, zirconium oxide, and absorbable phosphate glass and an absorbable polymeric matrix of a crystalline segmented copolymer made from at least two cyclic monomers selected from the group consisting of l-lactide, ε-caprolactone, trimethylene carbonate, glycolide, p-dioxanone, 1,5-dioxepan-2-one, and a morpholine-2,5-dione. Alternatively, the reinforcing braid or weft-knitted monofilament yarn is a composite of an inorganic microparticulate dispersed phase of at least one material selected from the group of barium sulfate, zirconium oxide, and absorbable phosphate glass, and wherein an absorbable polymeric matrix of a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione.

In another aspect, the present disclosure provides an absorbable and disintegratable, multicomponent, non-migrating (e.g., stationary) endoureteral intravesical degradable medical device and/or composition which is a construct of a fiber-reinforced elastomeric film designed with at least one position-retaining end, wherein the fiber reinforcement is a weft-knitted monofilament yarn and the reinforced construct is in the form of a tube with a central main component having a smaller diameter than that of the patient ureter and having at least one position-retaining end wherein the position-retaining end is a highly flexible extension of the central main tube, acquiring a loop shape with an open end parallel to the axis of the central main tube after insertion in the patient ureter and the loop can be made co-linear with the central main tube during insertion with an applicator, and wherein the film is formed of a crystalline segmented elastomeric high l-lactide copolymer and the monofilament is formed of a segmented l-lactide copolymer with at least one cyclic monomer selected from the group consisting of glycolide, ε-caprolactone and a morpholine-2,5-dione, and wherein the monofilament contains a microparticulate inorganic filler selected from the group of barium sulfate, zirconium oxide, and an absorbable phosphate glass.

In one aspect, a degradable medical device and/or composition is an intravesical device comprising a filament which has a longitudinal axis and which comprises an oriented bioabsorbable polymeric material, wherein polymer molecules within the bioabsorbable polymeric material have a helical orientation which is aligned with respect to the longitudinal axis of the filament, and wherein the intravesical device is at least partially bioabsorbed by a patient upon implantation or insertion of the intravesical device into the patient. In optional aspects, the following one or more features may further characterize the medical device: a) the filament is helically wound along at least a portion of the length of the intravesical device; b) the intravesical device comprises a plurality of said filaments, where optionally the plurality of filaments are helically wound along at least a portion of the length of the intravesical device, optionally a plurality of the filaments are helically wound in a first direction and a plurality of the filaments are helically wound in an opposite direction; c) the filament is a braided filament; a plurality of the braided filaments are braided and helically wound along at least a portion of the length of the intravesical device; d) the filament is a knitted filament; e) the plurality of filaments are knitted filaments; the orientated bioabsorbable polymeric material comprises either a single bioabsorbable polymer or a blend of bioabsorbable polymers; f) the oriented bioabsorbable polymeric material comprises a polymer selected from poly(alpha-hydroxy acid) homopolymers, poly(alpha-hydroxy acid) copolymers and blends thereof; g) the oriented bioabsorbable polymeric material comprises a polymer selected from polyglycolide, poly-L-lactide, poly-D-lactide, poly-DL-lactide, and blends thereof; h) the oriented bioabsorbable polymeric material has a crystallinity ranging from 0.1 to 20%; i) the filament comprises a core of oriented bioabsorbable polymeric material; j) the intravesical device is selected from a coronary vascular intravesical device, a peripheral vascular intravesical device, a urethral intravesical device, a ureteral intravesical device, a biliary intravesical device, a tracheal intravesical device, a gastrointestinal intravesical device and an esophageal intravesical device.

Optionally, an intravesical degradable medical device and/or composition is capable of maintaining patency, and providing at least an effective amount of an active agent, and remaining at the application site for at least two days, or 2-3 weeks, or has degraded after 7 weeks, or has degraded after 90 days, or has degraded by four months.

The present disclosure provides the following additional exemplary aspects:

In one aspect, the degradable medical device and/or composition is a biodegradable endoureteral intravesical degradable medical device and/or composition. The intravesical degradable medical device and/or composition comprises a tubular elastomeric film and a tubular fiber reinforcement, where the tubular elastomeric film is a single tube covering the tubular fiber reinforcement. The intravesical degradable medical device and/or composition has at least one position-retaining end and a central main tube having a smaller diameter than that of a patient ureter, wherein the at least one position-retaining end is an extension of the central main tube. The intravesical degradable medical device and/or composition is configured to be positioned in the patient ureter and extend from a patient kidney to a patient bladder and to be retained in position by the at least one position-retaining end. The film reinforces and impregnates the fiber-reinforcement, wherein the fiber-reinforcement comprises a monofilament coil disposed over a knitted or braided tube of a monofilament or multifilament yarn. The film and fiber reinforcement each comprise an absorbable crystalline segmented copolymer comprising at least one cyclic monomer. The film and fiber reinforcement alone are capable of maintaining ureteral patency.

The following options may further define the intravesical degradable medical device and/or composition: a) the at least one position-retaining end is a flexible extension of the central main tube, acquiring a goose-neck shape after insertion in the patient ureter but can be made colinear with the central main tube during insertion with an applicator; b) the tubular elastomeric film comprises a crystalline segmented copolymer of a polyethylene glycol and at least one cyclic monomer selected from the group consisting of l-lactide, epsilon-caprolactone, trimethylene carbonate, glycolide, p-dioxanone, 1,5-dioxepan-2-one, and a morpholine-2,5-dione; c) the tubular elastomeric film comprises a crystalline segmented copolymer of l-lactide and at least one cyclic monomer selected from the group consisting of glycolide, ε-caprolactone, trimethylene carbonate, p-dioxanone and 1,5-dioxepan-2-one; d) the monofilament coil comprises a crystalline segmented copolymer of at least two cyclic monomers selected from the group consisting of l-lactide, ε-caprolactone, trimethylene carbonate, glycolide, a morpholine-2,5-dione, p-dioxanone and 1,5-dioxepan-2-one; e) the monofilament coil comprises a composite comprising a polymeric matrix and an inorganic microparticulate dispersed phase contained within the matrix, the matrix comprising a crystalline segmented copolymer and the inorganic microparticulate dispersed phase comprising at least one material selected from the group consisting of barium sulfate, zirconium oxide, and absorbable phosphate glass; f) the monofilament coil comprises a composite comprising a polymeric matrix and an inorganic microparticulate dispersed phase contained within the matrix, the matrix comprising a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione and the inorganic microparticulate dispersed phase comprising at least one material selected from the group consisting of barium sulfate, zirconium oxide, and absorbable phosphate glass; g) the monofilament coil comprises a composite comprising a polymeric matrix and an inorganic microparticulate dispersed phase contained within the matrix, the matrix comprising a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione; h) the fiber-reinforcement comprises a monofilament coil and a braided tube of a multifilament yarn, where optionally, 1) the tubular elastomeric film comprises a crystalline segmented copolymer of polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione; 2) the tubular elastomeric film comprises a crystalline segmented copolymer of I-lactone and at least one cyclic monomer selected from the group consisting of glycolide, ε-caprolactone, trimethylene carbonate, p-dioxanone, and 1,5-dioxepan-2-one, and a morpholine-2,5-dione; 3) the monofilament coil comprises a crystalline segmented copolymer of at least two cyclic monomers selected from the group consisting of l-lactide, epsilon-caprolactone, trimethylene carbonate, glycolide, a morpholine-2,5-dione, p-dioxanone, 1,5-dioxepan-2-one; 4) the monofilament coil comprises a composite comprising a polymeric matrix and an inorganic microparticulate dispersed phase contained within the matrix, the matrix comprising a crystalline segmented copolymer of at least two cyclic monomers selected from the group consisting of l-lactide, ε-caprolactone, trimethylene carbonate, glycolide, p-dioxanone, 1,5-dioxepan-2-one, and a morpholine-2,5-dione and the inorganic microparticulate dispersed phase comprising at least one material selected from the group consisting of barium sulfate, zirconium oxide, and absorbable phosphate glass; 5) the monofilament coil comprises a composite comprising a polymeric matrix and an inorganic microparticulate dispersed phase contained within the matrix, the matrix comprising a crystalline segmented copolymer of a polyethylene glycol and at least two cyclic monomer selected from the group consisting of l-lactide, ε-caprolactone, trimethylene carbonate, glycolide, p-dioxanone, 1,5-dioxepan-2-one, and a morpholine-2,5-dione and the inorganic microparticulate dispersed phase comprising at least one material selected from the group consisting of barium sulfate, zirconium oxide, and absorbable phosphate glass; 6) the multifilament yarn comprises a crystalline segmented copolymer of a polyethylene glycol and at least one cyclic monomer selected from the group consisting of l-lactide, trimethylene carbonate, ε-caprolactone, glycolide, p-dioxanone, a morpholine-2,5-dione and 1,5-dioxepan-2-one; 7) the multifilament yarn comprises a crystalline segmented copolymer of l-lactide and at least one cyclic monomer selected from the group consisting of glycolide, ε-caprolactone, trimethylene carbonate, p-dioxanone, 1,5-dioxepan-2-one and a morpholine-2,5-dione; j) the monofilament coil is disposed over a tube of weft-knitted monofilament yarn, where optionally 1) the tubular elastomeric film comprises a crystalline segmented copolymer of polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione; 2) the tubular elastomeric film comprises a crystalline segmented copolymer of l-lactide and at least one cyclic monomer selected from the group consisting of glycolide, ε-caprolactone, trimethylene carbonate, p-dioxanone and 1,5-dioxepan-2-one, and a morpholine-2,5-dione; 3) the monofilament yarn comprises a crystalline segmented copolymer of at least two cyclic monomers selected from the group consisting of l-lactide, ε-caprolactone, trimethylene carbonate, glycolide, a morpholine-2,5-dione, p-dioxanone, and 1,5-dioxepan-2-one; 4) the monofilament yarn comprises a crystalline segmented copolymer of a polyethylene glycol and at least one cyclic monomer selected from the group consisting of l-lactide, trimethylene carbonate, ε-caprolactone, glycolide, p-dioxanone, a morpholine-2,5-dione, and 1,5-dioxepan-2-one; 5) the monofilament yarn comprises a composite comprising a polymeric matrix and an inorganic microparticulate dispersed phase contained within the matrix, the matrix comprising a crystalline segmented copolymer of at least two cyclic monomers selected from the group consisting of l-lactide, ε-caprolactone, trimethylene carbonate, glycolide, p-dioxanone, 1,5-dioxepan-2-one, and a morpholine-2,5-dione and the inorganic microparticulate dispersed phase comprising at least one material selected from the group consisting of barium sulfate, zirconium oxide, and absorbable phosphate glass; and 6) the monofilament yarn comprises a composite comprising a polymeric matrix and an inorganic microparticulate dispersed phase contained within the matrix, the matrix comprising a crystalline segmented copolymer of a polyethylene glycol and at least one cyclic monomer selected from the group consisting of l-lactide, ε-caprolactone, trimethylene carbonate, glycolide, p-dioxanone, 1,5-dioxepan-2-one, and a morpholine-2,5-dione and the inorganic microparticulate dispersed phase comprising at least one material selected from the group consisting of barium sulfate, zirconium oxide, and absorbable phosphate glass; k) the intravesical device is capable of maintaining patency and remaining at an application site for at least two days; l) the intravesical device is capable of maintaining patency, eluting at least one active agent, and remaining at an application site for two to four months; and m) the at least one position-retaining end contains at least 4 percent by weight of at least one powdered radiopacifier selected from the group consisting of barium sulfate, zirconium oxide, and bismuth subcarbonate.

An intravesical degradable medical device and/or composition can elute one or more active agents. In an aspect, a release profile or elution profile of an active agent from an intravesical device can be measured in vitro under sink conditions. In an aspect, an elution profile of at least one active agent can comprise a burst phase. In an aspect, about 10% (w/w) to about 50% (w/w) of at least one active agent is released in the burst phase. In an aspect, about 10% (w/w) to about 50% (w/w) of at least one active agent is released in the burst phase within two weeks. In an aspect, about 10% (w/w) to about 50% (w/w) of at least one active agent is released in the burst phase within one week. In an aspect, the elution profile of at least one active agent exhibits release of less than about 10% (w/w) of the active agent over a two week period. In an aspect, an elution profile of at least one active agent exhibits release of less than about 10% (w/w) of the active agent over a one week period.

In an aspect, an intravesical degradable medical device and/or composition releases at least one active agent for greater than one week. In an aspect, an intravesical degradable medical device and/or composition releases at least one active agent for greater than two weeks. In an aspect, an intravesical degradable medical device and/or composition releases at least one active agent for greater than three weeks. In an aspect, an intravesical degradable medical device and/or composition releases at least one active agent for greater than four weeks. In an aspect, an intravesical degradable medical device and/or composition releases at least one active agent for greater than six weeks. In an aspect, an intravesical degradable medical device and/or composition releases at least one active agent for greater than eight weeks. In an aspect, an intravesical degradable medical device and/or composition releases greater than about 70% (w/w) of at least one active agent over two weeks. In an aspect, an intravesical degradable medical device and/or composition releases greater than about 70% (w/w) of at least one active agent over four weeks. In an aspect, an intravesical degradable medical device and/or composition releases about greater than 70% (w/w) of at least one active agent over six weeks. In an aspect, an intravesical degradable medical device and/or composition releases greater than about 70% (w/w) of at least one active agent over eight weeks. In an aspect, an intravesical degradable medical device and/or composition releases greater than about 70% (w/w) of at least one active agent over twelve weeks. In an aspect, an intravesical degradable medical device and/or composition releases greater than about 70% (w/w) of at least one active agent over sixteen weeks. In an aspect, an intravesical degradable medical device and/or composition releases about 50% (w/w) to about 60% of at least one active agent over two weeks. In an aspect, an intravesical degradable medical device and/or composition releases about 50% (w/w) to about 60% (w/w) of at least one active agent over four weeks. In an aspect, an intravesical degradable medical device and/or composition releases about 50% (w/w) to about 60% (w/w) of at least one active agent over six weeks. In an aspect, an intravesical degradable medical device and/or composition releases about 50% (w/w) to about 60% (w/w) of at least one active agent over eight weeks. In an aspect, an intravesical degradable medical device and/or composition releases about 50% (w/w) to about 60% (w/w) of at least one active agent over twelve weeks. In an aspect, an intravesical degradable medical device and/or composition releases about 50% (w/w) to about 60% (w/w) of at least one active agent over sixteen weeks. In an aspect, an intravesical degradable medical device and/or composition releases about 40% (w/w) to about 50% (w/w) of at least one active agent over two weeks. In an aspect, an intravesical degradable medical device and/or composition releases about 40% (w/w) to about 50% (w/w) of at least one active agent over four weeks. In an aspect, an intravesical degradable medical device and/or composition releases about 40% (w/w) to about 50% (w/w) of at least one active agent over six weeks. In an aspect, an intravesical degradable medical device and/or composition releases about 40% (w/w) to about 50% (w/w) of at least one active agent over eight weeks. In an aspect, an intravesical degradable medical device and/or composition releases about 40% (w/w) to about 50% (w/w) of at least one active agent over twelve weeks. In an aspect, an intravesical degradable medical device and/or composition releases about 40% (w/w) to about 50% (w/w) of at least one active agent over sixteen weeks. In an aspect, an intravesical device releases about 20% (w/w) to about 40% (w/w) of at least one active agent over two weeks. In an aspect, an intravesical degradable medical device and/or composition releases about 20% (w/w) to about 40% (w/w) of at least one active agent over four weeks. In an aspect, an intravesical degradable medical device and/or composition releases about 20% (w/w) to about 40% (w/w) of at least one active agent over six weeks. In an aspect, an intravesical degradable medical device and/or composition releases about 20% (w/w) to about 40% (w/w) of at least one active agent over eight weeks. In an aspect, an intravesical degradable medical device and/or composition releases about 20% (w/w) to about 40% (w/w) of at least one active agent over twelve weeks. In an aspect, an intravesical degradable medical device and/or composition releases about 20% (w/w) to about 40% (w/w) of at least one active agent over sixteen weeks. In an aspect, an intravesical degradable medical device and/or composition releases about 5% (w/w) to about 20% of at least one active agent over two weeks. In an aspect, an intravesical degradable medical device and/or composition releases about 5% (w/w) to about 20% (w/w) of at least one active agent over four weeks. In an aspect, an intravesical degradable medical device and/or composition releases about 5% (w/w) to about 20% (w/w) of at least one active agent over six weeks. In an aspect, an intravesical degradable medical device and/or composition releases about 5% (w/w) to about 20% (w/w) of at least one active agent over eight weeks. In an aspect, an intravesical degradable medical device and/or composition releases about 500 ug to about 1000 ug of at least one active agent in a day. In an aspect, an intravesical degradable medical device and/or composition releases about 300 ug to about 500 ug of at least one active agent in a day. In an aspect, an intravesical degradable medical device and/or composition releases about 200 ug to about 300 ug of at least one active agent in a day. In an aspect, an intravesical degradable medical device and/or composition releases about 100 ug to about 200 ug of at least one active agent in a day. In an aspect, an intravesical degradable medical device and/or composition releases about 1 ug to about 100 ug of at least one active agent in a day.

The following are some additional the aspects of the present disclosure:
1) A degradable medical device and/or composition medical device and/or composition comprising a containment layer that at least partially encases a degradable medical device and/or composition, the degradable medical device and/or composition being at least partially biodegradable when the medical device and/or composition is implanted in a subject, the containment layer being either nonbiodegradable or biodegradable, where the containment layer serves as a container for the medical device and/or composition as that medical device and/or composition degrades in vivo.
2) The degradable medical device and/or composition of aspect 1 wherein the device provides structural support within a subject.
3) The degradable medical device and/or composition of aspects 1-2 wherein the device is a intravesical device.
4) The degradable medical device and/or composition of aspects 1-3 wherein the intravesical device is an endoureteral intravesical device.
5) The degradable medical device and/or composition of aspects 1-4 wherein the containment layer is a coating on the medical device and/or composition.
6) The degradable medical device and/or composition of aspect 5 wherein the coating is hydrophilic.
7) The degradable medical device and/or composition of aspects 5-6 wherein the coating is biodegradable, but the coating degrades more slowly than the medical device and/or composition.
8) The degradable medical device and/or composition of aspects 5-7 wherein the coating has a thickness of greater than 20 microns.
9) The degradable medical device and/or composition of aspects 5-8 wherein the coating has a thickness of greater than 40 microns.
10) The degradable medical device and/or composition of aspects 5-9 wherein the coating has a thickness of greater than 60 microns.
11) The degradable medical device and/or composition of aspects 5-10 wherein the coating has a thickness of greater than 80 microns.
12) The degradable medical device and/or composition of aspects 5-11 wherein the coating has a thickness of greater than 100 microns.
13) The degradable medical device and/or composition of aspects 5-12 wherein the coating has a thickness of greater than 120 microns.
14) A degradable medical device and/or composition comprising a containment layer that is at least partially encased by a medical device and/or composition, where the containment layer at least partially encases a hollow center of the medical device and/or composition, the degradable medical device and/or composition being at least partially biodegradable when the degradable medical device and/or composition is implanted in a subject, the containment layer being either nonbiodegradable or biodegradable, where the containment layer provides a barrier between the degradation product that forms during the biodegradation of the degradable medical device and/or composition and the hollow space of the medical device and/or composition.

It is to be understood that the terminology used herein is for the purpose of describing specific aspects only and is not intended to be limiting. It is further to be understood that unless specifically defined herein, the terminology used herein is to be given its traditional meaning as known in the relevant art.

Reference throughout this specification to “one aspect” or “an aspect” and variations thereof means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects. Any of the degradable medical device and/or composition aspects disclosed herein may include a active agent, e.g., a active agent, or a prophylactic agent, as part of the medical device and/or composition.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an aspect that includes all of the associated items or ideas and one or more other alternative aspects that include fewer than all of the associated items or ideas.

Unless the context requires otherwise, throughout the specification and claims that follow, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. The term “comprising” may also include the limitations associated with the use of “consisting of” or “consisting essentially of”.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed invention. A ‘consisting essentially of claim occupies a middle ground between closed claims that are written in a ‘consisting of format and fully open claims that are drafted in a ‘comprising’ format. Optional additives as defined herein, at a level that is appropriate for such additives, and minor impurities are not excluded from a composition by the term “consisting essentially of”.

When a composition, a process, a structure, or a portion of a composition, a process, or a structure, is described herein using an open-ended term such as “comprising,” unless otherwise stated the description also includes an embodiment that “consists essentially of” or “consists of” the elements of the composition, the process, the structure, or the portion of the composition, the process, or the structure.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, a mammalian subject is a human. The term “patient” includes human and veterinary subjects.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the aspects.

In the description, certain specific details are set forth to provide a thorough understanding of various disclosed aspects. However, one skilled in the relevant art will recognize that aspects may be practiced without one or more of these specific details, or with other methods, components, materials, etc.

The Examples and preparations provided below further illustrate and exemplify the medical devices and/or compositions of the present invention and methods of preparing such devices. It is to be understood that the scope of the present invention is not limited in any way by the scope of the following Examples and preparations. In fact, unless the context indicates otherwise, when a specific polymer is used in an Example, this polymer is exemplary only and may, according to the present invention, be replaced with an alternative polymer. Also, when degradation times and properties are exemplified, it is to be understood that these values are approximations, and that other values would be obtained using different starting materials. The starting materials and various reactants utilized or referenced in the examples may be obtained from commercial sources, or are readily prepared from commercially available organic compounds, using methods well-known to one skilled in the art. Thus, the following examples are illustrative of aspects of the invention, and are not to be construed as a limitation thereon.

### EXAMPLES

### Example 1 Preparation of Coil

A 1-liter stainless steel kettle with a 3-neck glass lid equipped with an overhead mechanical stirring unit, vacuum adapter, and nitrogen inlet was set up. The kettle was evacuated to a pressure of about 0.5 mm Hg and then purged with nitrogen. The kettle was charged with 9.15 g of paxTMC-1, which was pre-dried by heating it to 220° C. paxTMC-1 was prepared by combining trimethylene carbonate (TMC) and trimethylolpropane (TMP) at a TMC:TMP molar ratio of 15:1, in the presence of a tin catalyst such as stannous octanoate, with heating and stirring. Also added to the kettle was glycolide (313.8 g, 2.705 mol), ε-caprolactone (132.1 g, 1.159 mol) and a radiopacifier. In one aspect, the radiopacifier was barium sulfate microparticles (245 g, having a diameter between 1 and 4 microns. The apparatus was lowered into an oil bath, and its contents are placed under vacuum at 40° C. for 1 hour, and then the system was purged with nitrogen. The temperature of the oil bath was increased to 95° C. and the kettle contents are mixed thoroughly. After a homogenous fluid composition was attained, a 0.2 M toluene solution of stannous octanoate (2.576 mL, 5.152×10-4 moles stannous octanoate) was added. The temperature of the oil bath was increased to 180° C. whereupon the polymerization reaction takes place and stirring was continued for as long as possible. After stirring was not possible (due to high viscosity), the reaction product was maintained at 180° C. for 7 hours.

The kettle was allowed to cool to room temperature and then lowered into a cold bath to freeze the polymer. The frozen polymer was removed from the kettle and ground up. The ground material was sieved to provide a powder having a desired maximum particle size. The sieved powder was transferred to a 2-liter pear shaped glass flask and placed on a Büchi rotavapor. After obtaining a vacuum of 0.25 mm Hg, the flask was lowered into an oil bath and the temperature was increased to 40° C. After 2 hours at 40° C. the temperature of the oil bath was increased to 80° C., and after 1 hour at 80° C. the temperature was increased to 110° C. The temperature was maintained at 110° C. for 4 hours.

The identity, particle size and amount of the radiopacifier may be selected to provide a desired impact on the device and/or composition degradation profile. In general, a sufficient amount of radiopacifier was included in the composition to allow the composition to be visualized. Extra radiopacifier, i.e., an amount of radiopacifier that is above and beyond the amount needed for visualization, may be included in the composition in order to impact the degradation rate of the composition. While not intending to be bound by theory, it is believed that extra radiopacifier creates stress points within the composition that encourages degradation. If, for example, at least 20 weight percent of radiopacifier should be present in order to visualize the medical device and/or composition, then in various aspects the present disclosure provides compositions that contain at least 5%, or at least 10% (2 additional weight percent), or at least 15%, or at least 20% (4 additional weight percent), or at least 25%, or at least 30% (6 additional weight percent), or at least 35%, or at least 40% (8 additional weight percent), or at least 45%, or at least 50% (10 additional weight percent), or at least 55%, or at least 60%, or at least 65%, or at least 70%, or at least 75% extra radiopacifier.

In addition to selecting the amount of radiopacifier, one can select the particle size of the radiopacifier. Radiopacifier particles of various sizes and size distributions are commercially available from, e.g., Sigma-Aldrich, St. Louis Mo. The radiopacifier, such as barium sulfate, may have a nominal particle diameter of from about 1.0 to about 20 microns. In aspects, the radiopacifier used in the medical devices and/or compositions of the present disclosure had a nominal particle size of at least 1.0, or 2.0, or 3.0, or 4.0, or 5.0, or 6.0, or 7.0, or 8.0, or 9.0, or 10.0, or 11.0, or 12.0, or 13.0, or 14.0, or 15.0, or 16.0, or 18.0, or 19.0 or 20.0, each value in units of microns. Optionally, the maximum particle size may be 20.0, or 19.0, or 18.0, or 17.0, or 16.0, or 15.0, or 14.0, or 13.0, or 12.0, or 11.0, or 10.0, or 9.0, or 8.0, or 7.0, or 6.0, or 5.0, or 4.0, or 3.0, or 2.0, or 1.0, again with each value being in units of microns. In general, larger particles impart a faster degradation profile to the degradable medical device and/or composition, since larger particles impart larger stress concentrations within the resulting fiber, leading to earlier loss in tensile properties and ultimately earlier fragmentation. A higher weight percentage of radiopacifier particle also contributes to a faster degradation profile.

In general, a lower concentration of a higher density radiopacifer may be employed. Barium sulfate has a density of 4.5 g/cm3, and so more barium sulfate must generally be loaded into a degradable medical device and/or composition in order to achieve effective visualization compared to, e.g., tantalum oxide, tungsten metal and zirconium oxides which are examples of radiopacifier that are denser than barium sulfate. Bi₂O₂(CO₃), i.e., bismuth subcarbonate, BiOCl, i.e., bismuth oxychloride, and Bi₂O₃, i.e., bismuth trioxide, in particulate form, are other radiopacifiers that may be incorporated into medical devices and/or compositions of the present disclosure.

In one aspect, the radiopacifier is present during the polymerization process, while in another aspect the radiopacifier is added to the pre-formed polymer.

### Example 2

### Melt-Spinning and Properties of Radiopaque Monofilaments Using Polymer from Example 1 and its Processing into a Coiled Scaffold (CS)

A single screw extruder with four zones was used to extrude Example 1 polymer into monofilament. The Example 1 polymer 1 was extruded using a 0.6 mm die. A 325 line per inch filter pack was used. Zone 1 was maintained at 100° C., zone 2 was maintained at 175° C., zone 3 was maintained at 212° C., and zone 4/Spin Pack were maintained at 214° C. The metering pump was operated at 8 rpm while the take up roll was set at 40-60 rpm. The collected monofilament may have diameters between 0.58 mm and 0.61 mm. The fiber was drawn at 4.5× in the first stage at 55° C. and 0.5× in the second stage at 70° C., resulting in a diameter of 0.30 mm to 0.33 mm. The free shrinkage was about 8.85% to 10.43% at 50° C. The fiber was relaxed one half the free shrinkage plus 2% at 70° C. The resulting fiber may have a maximum load of about 13N and was dimensionally stable.

The processed radiopaque monofilament was then coiled in a helical manner around a 0.55″ diameter Teflon cord which maintains the inner diameter of the coil scaffold. The monofilament was wrapped around the Teflon cord at 33 to 35 coils per inch.

### Example 3

### Synthesis and Characterization of a Triaxial, Segmented Glycolide Copolymer for Use in Preparing Knitted Scaffolds

A reaction apparatus including a 1 L stainless steel kettle with a 3-neck glass lid equipped with an overhead mechanical stirring unit, vacuum adapter, and nitrogen inlet was set up. After obtaining a vacuum of 0.5 mmHg, the apparatus was purged with nitrogen. An initial charge of paxTMC-1 (16.0 g, as described in Example 1), ε-caprolactone (38.6 g, 0.3382 moles), and glycolide (745.4 g, 6.4262 moles) were added to the kettle. The apparatus was then lowered into an oil bath. The kettle and the contents were heated to 110° C. and mixed under positive nitrogen pressure. Once the polymeric initiator appears to be thoroughly dissolved into the monomer, a 0.2 M toluene solution of stannous octanoate (0.966 ml, 1.933×10⁻⁴ moles) was added. The temperature was increased to 180° C. Stirring was stopped when the resulting polymer mixture gets too viscous to stir. The reaction was maintained at 180° C. for 5 hours. The polymer was frozen, removed and ground. The ground material was sieved. Sieved polymer was transferred to a 2 L pear shaped glass flask and placed on a Büchi rotavapor. After obtaining a vacuum of 0.5 mmHg, the flask was lowered into an oil bath. The temperature was increased to 40° C. After 2 hours at 40° C., the temperature of the oil bath was increased to 80° C. After 1 hour at 80° C., the temperature was increased to 110° C. Temperature was maintained at 110° C. for 4 hours.

### Example 4

### Melt-Spinning and Properties of Multifilament Yarn from Example 3 Material and its Processing to a Knitted Scaffold (KS)

A single screw extruder with five zones was used to extrude a polymer into multifilament. The polymer from Example 3 was extruded using a 20 hole die with 0.018″ diameter holes. A 400 line per inch filter pack was used. Zone 1 was maintained at 190° C., zone 2 was maintained at 210° C., zone 3 was maintained at 222° C., zone 4/pump were maintained at 228° C., and zone 5/spin pack were maintained at 228° C. A 0.584 cc/rev Zenith metering pump was operated at 6.0 rpm while the denier control roll was set to a linear speed of 315 meters/minute. The fiber was then oriented over three high speed godets traveling at 320, 465, 480 M/minute and heated to 60° C., 80° C., and 26° C., respectively. The collected multifilament was then reoriented at speeds of 250 M/minute to 280 M/minute, and at a temperature of 100° C. The resulting fiber may have a tenacity of 3.26 and a denier of 80.4. The processed multifilament was then plied once to generate a 40 filament fiber and then weft knitted using a lamb circular knitter onto the coiled scaffold from Example 2 in a continuous manner. A ⅞″ knitting cylinder with 12 course gauge needles was used to form a knitted scaffold over the coiled scaffold.

### Example 5

### Synthesis and Characterization of a Triaxial, Segmented l-Lactide Copolymer for Use as a Reinforced Composite Matrix (CM)

A reaction apparatus including a 4 L stainless steel reactor equipped with an overhead mechanical stirring unit, vacuum adapter, and nitrogen inlet was assembled. After obtaining a vacuum of less than 0.5 mmHg, the apparatus was purged with nitrogen. Oil was heated and circulated through the jacketed reactor to control the temperature. An initial charge of glycolide (254.9 g, 2.1976 moles), trimethylene carbonate (348.7 g, 3.4185 moles), predried triethanolamine (3.0319 g, 2.0348×10⁻² moles), stannous octanoate (354.5 mg, 8.752×10⁻⁴ moles), and ε-caprolactone (974.3 g, 8.5463 moles) was added a 2 L flask and dried under high vacuum for 1.25 hours at 40° C. The flask contents were then added to the 4 L reactor. The system was then purged with nitrogen. The temperature of the oil was increased to 175° C. and the contents mixed thoroughly for 6.5 hours and then the temperature was reduced. Once mixed, the final charge, of glycolide (226.6 g, 1.9534 moles) and l-Lactide (1195.5 g, 8.3021 moles) were added. The temperature of the oil was then increased to 135° C. and maintained for 19 hours.

The resulting polymer was removed and dissolved at a concentration of 4 milliliters per 1 gram in dichloromethane (DCM) so that the polymer precipitated out in −60° C. isopropyl alcohol (IPA) and any monomer was dissolved and rinsed away. The polymer was then allowed to dry to a constant weight.

### Example 6

### Assembling a Composite Intravesical Device Construct

Preparation of polymer matrix solution—A polymer solution containing polymer from Example 5, polyethylene glycol (M_{w}=4600) and acetone was prepared by addition of 1600 milliliters of acetone to one 64-ounce jar, followed by addition of 16.0 grams of PEG 4600 and 144.0 grams of purified SVG-12. The solution was enclosed and brief heating was used to facilitate dissolution. The jar was placed on automatic rolling apparatus until complete dissolution was reached.

Continuous impregnating of knitted core—The dry, knitted core was impregnated with a polymer matrix of Example 5 polymer and PEG 4600 using a continuous matrix-impregnating process that involves the continuous movement of the knitted core material through a 0.75-liter bath of polymer solution. The knitted core was unspooled from the beginning of the impregnating apparatus and immediately fed into the bath of polymer solution, where two in-line submerged pulleys keep the scaffold material submerged for the length of the bath. As the impregnated material exits the bath, it was passed through an air-circulating drying tube heated to 40° C., then a stainless-steel element heated to 50° C., and then the impregnated material was spooled onto a final take-up spool.

Shape-forming of impregnated, knitted core—The impregnated material was wrapped onto racks equipped with two parallel stainless steel bars of 0.5 inch diameter, which can be adjusted for separation distance to control final intravesical device length. The newly-impregnated, knitted core was wrapped onto these racks in a continuous fashion. The racks are annealed at 130° C. for 30 minutes, and then the racks were allowed to cool to room temperature in a laminar flow hood. Multiple intravesical devices were removed from each rack by cutting the scaffolding material at appropriate positions along interior positions of the separation rods of the shape-forming racks. These intravesical devices, which still contained the Teflon core, were modified by addition of a UVJ marker to each intravesical device stem within one centimeter of what would ultimately become the proximal loop of each intravesical device. Then, the proximal loops of all intravesical devices were given an additional coating by hand-dipping each proximal loop into 150 milliliters of a 10% (w/v; 9.3% SVG-12, 0.7% PEG 4600) polymer solution of SVG-12 and PEG 4600 in acetone. Intravesical devices were hung by distal loops in a laminar flow hood to dry. Then, the Teflon cores were removed from each intravesical device by securing one end of the Teflon core to position-fixed vise-grip pliers as the opposite end of the Teflon core was stretched using a second set of vise-grip pliers. A clean cut was made in the stretched Teflon core at the secured end, and then the Teflon core of reduced diameter was pulled through the intravesical device and discarded. Finally, each intravesical device was trimmed to the appropriate specifications.

### Example 7

### Assembling a Composite Ureteral Intravesical Device Construct

Preparation of polymer solution. A polymer solution was prepared by combining 16.0 grams of polyethylene glycol (PEG 4600; M_{w}=4600), 1600 milliliters of acetone, and 144.0 grams of purified Example 5 polymer in a jar. The solution was enclosed and brief heating was used to facilitate dissolution. The jar was placed on automatic rolling apparatus until complete dissolution was reached.

The dry, knitted scaffold from Example 4 was impregnated with the polymer solution described above using a continuous impregnating process that involved the continuous movement of the knitted core material through a 0.75-liter bath of polymer solution. The scaffold was unspooled and fed into a bath of coating solution, where two in-line submerged pulleys keep the scaffold material submerged for the length of the bath. As the impregnated material exits the bath, it passes through an air-circulating drying tube heated to 40° C., then a stainless-steel element heated to 50° C., and then the impregnated material was spooling onto a final take-up spool. This process was repeated in order to provide a thicker coating on the scaffold, where the thicker coating has a smooth surface.

The impregnated knitted scaffold was wrapped onto racks equipped with two parallel stainless steel bars of 0.5 inch diameter, which can be adjusted for separation distance to control final intravesical device length. The newly-impregnated, knitted scaffold was wrapped onto these racks in a continuous fashion. The racks were annealed at 130° C. for 30 minutes, and then the racks were allowed to cool to room temperature in a laminar flow hood.

Multiple intravesical devices were removed from each rack by cutting the scaffolding material at appropriate positions along interior positions of the separation rods of the shape-forming racks. These intravesical devices, which still contain the Teflon core, were modified by addition of a UVJ marker to each intravesical device stem within one centimeter of what would ultimately become the proximal loop of each intravesical device.

The proximal loops of the intravesical devices were given an additional coating by using an MTS Synergie testing apparatus to mechanically dip the proximal end of the intravesical device into a coating solution in a controlled manner and using multiple cycles. The distal end of the intravesical device was attached to the vertical fixture on the MTS testing apparatus, which was programmed to dip the intravesical device into a 100 mL graduated cylinder containing 100 mL of the coating solution. The programmed procedure lowered the intravesical device into the cylinder to the 20 mL marking and immediately raised the intravesical device out of the cylinder. The MTS apparatus paused for a time sufficient to dry the coating until it reached a non-tacky state, about 30-300 seconds as the intravesical device was suspended above the coating solution, and then the dipping procedure was repeated, with the exception that the intravesical device was lowered to the 40 mL marking. The MTS program performed two final dipping cycles, wherein the intravesical device was lowered to the 60 mL and then to 80 mL mark. This resulted in an exterior coating layer that has a thickness gradient, wherein the thickest layer of coating was located on the proximal loop. This ensured that the proximal loop was reinforced with more coating material than the rest of the intravesical device so that the proximal loop does not degrade prematurely.

Intravesical devices were hung by distal loops in a laminar flow hood to dry. Then, TEFLON™ PTFE cores were removed from each intravesical device by securing one end of the TEFLON core to position-fixed vise-grip pliers as the opposite end of the TEFLON was stretched using a second set of vise-grip pliers. A clean cut was made in the stretched TEFLON core at the secured end, and then the Teflon core of reduced diameter was pulled through the intravesical device and discarded. Finally, each intravesical device was trimmed to the appropriate specifications.

The resulting intravesical devices had a fast-degrading internal structure (knitted scaffold) and a hydrophilic, exterior containment layer that degraded more slowly than the internal structure. The internal structure degraded and was completely eliminated within 4 days, or 1 week, or 2-4 weeks, or within 2-7 weeks, or as long as 90 days, or as long as six months, whereas the longer lasting containment layer was still present after the internal structure was completely degraded, such that the containment layer was present for at least 4 weeks before it was degraded and excreted. As a result, the containment layer functioned to retain the internal structure and its degradation products until complete degradation and excretion occurred. This function of the containment layer prevented degradation products of fast-degrading material from entering the kidney which can result in complications requiring removal of remnant material that can cause blockage. This was an example of a containment layer that was used in connection with a medical device and/or composition, e.g., any of the degradable medical devices and/or compositions disclosed herein, to provide managed degradation when the device is implanted into a subject.

### Example 8

### Containment Layer that May be Used in Connection with a Degradable Medical Device and/or Composition to Provide Managed Degradation

This example provides a bioabsorbable intravesical device comprising an inner containment layer, an intermediate monofilament coil in a helical configuration, a weft-knit mesh on the exterior of the monofilament coil, and an exterior, hydrophilic containment layer applied as an outer layer that also penetrates and fills voids and empty spaces between material of the other three components—particularly the intermediate coil and weft-knit mesh components. The exterior containment layer degraded more slowly than the monofilament coil and the weft-knit mesh components, resulting in a containment layer that functioned to retain degradation products of the coil and mesh that degrade within 2-4 weeks.

The first step in constructing the multi-component bioabsorbable ureteral intravesical device involved applying an inner coating layer to a monofilament cord of polytetrafluoroethylene (PTFE) having a diameter of approximately 0.055 inches. The inner coating layer functioned as the inner containment layer. The PTFE monofilament cord was coated according to the drawing process disclosed in Example 1, however PTFE monofilament cord was substituted for the knitted core, a polymer solution of 15% (weight per volume) polymer in acetone was used and the monofilament was coated two times using the procedure described. The monofilament was fed through the polymer solution at a rate not exceeding 6 meters per second (m/s) to ensure that a sufficient thickness of polymeric coating was applied to the surface of the PTFE that the coating can function as a containment layer. This is an example of a containment layer that may be used in connection with a degradable medical device and/or composition, e.g., any of the degradable medical devices and/or compositions disclosed herein, to provide managed degradation when the device is implanted into a subject.

### Example 9

### Caps that May be Used in Connection with a Degradable Medical Device and/or Composition to Manage Degradation

A cap may be added to a intravesical device, where the cap provides some or all of a containment layer. The cap was located at either end of the intravesical device, and bridges across the open lumen of the intravesical device, so as to enclose the capped end of the intravesical device.

A cap may be placed on the intravesical device as exemplified herein. The cap was formed after the removal of the TEFLON monofilament cord from the intravesical device. After the cord was removed, an end of the intravesical device was dipped into a solution of degradable polymer. This solution was sufficiently viscous such that, after the intravesical device has been removed from the solution, some amount of solution bridged across the open lumen at the end of the intravesical device, and upon solvent removal through evaporation, left a polymer film that covers the dipped end of the intravesical device. This polymer film was the cap. This process may be repeated several times as needed in order to build up a desired thickness for this cap.

As an alternative, a cap may be prepared from a degradable mesh, and the mesh was adhered to the end of the intravesical device so as to form the cap. In this aspect, the cap was porous, such as is desirable when the intravesical device is a urethral intravesical degradable medical device and/or composition. However, the cap need not be porous, or it may only become porous after it has been implanted.

These are examples of caps that may be used in connection with a degradable medical device and/or composition, e.g., any of the degradable medical devices and/or compositions disclosed herein, to provide managed degradation when the device is implanted into a subject.

### Example 10

### Longitudinal Slitting of Coil Component to Create a Predictable Failure Point in the Coil Component

Example 1 polymer was prepared into a stabilized monofilament through melt extrusion and oriented to create a fiber with diameter of about 0.3 mm, which was further processed through a coiling process onto a 0.055″ diameter Teflon cord to maintain the inner diameter of the coil. After coiling and while still on the Teflon cord, the coil component passed along a fixed knife edge cutter to impart a longitudinal slit of approximately 0.02 mm in depth transverse to the fiber axis and axially along the coil length. The longitudinal slit reduced the initial tensile strength by a small amount, e.g., about 5%. The tensile strength was measured by upwrapping the monofilament from the cord and then placing a segment of monofilament into a tensile strength tester. As a consequence of the cutting, there was obtained a coiled, slitted scaffold having 33 to 35 coils per inch of glycolide copolymer monofilament and a transverse slit along the length of the scaffold. In vivo, the degradable medical device and/or composition fractured along the slit points, and degradation occurred initially at the locations of the slit.

The depth of the slit was selected in view of the diameter of the monofilament. In various aspects, the depth of the slit may be 1% or 2% or 3% or 4% or 5%, or 6%, or 7%, or 8%, or 9%, or 10%, or 11%, or 12%, or 13%, or 14%, or 15%, or 16%, or 17%, or 18%, or 19%, or 20%, or 21%, or 22%, or 23%, or 24%, or 25%, or 26%, or 27%, or 28%, or 29%, or 30%, or 31%, or 32%, or 33%, or 34%, or 35% of the diameter of the monofilament, including ranges selected from these percentage values.

This is an example of a slit that may be created in a degradable medical device and/or composition, e.g., any of the degradable medical devices and/or compositions disclosed herein, to provide managed degradation when the device was implanted into a subject.

### Example 11

### Preparation of a Degradable Ureteral Intravesical Device Containing a Longitudinal Defect to Provide Predictable Degradation Failure Points

Glycoprene polymer (prepared from 93% glycolide, 5% caprolactone, and 3% trimethylene carbonate) formed into a multifilament fiber and having a tenacity of 3.26 and 4.0 denier per filament was prepared through a melt extrusion process. A Lamb circular weft knitter was used to form a knit of Glycoprene multifilament fiber over the coiled and slitted scaffold from Example 10 in a continuous manner. A ⅞″ knitting cylinder with 12 course gauge needles was used to form the knitted scaffold over the coiled and slitted scaffold.

In a separate container, a polymer solution containing Example 5 polymer described above, polyethylene glycol (M_{w}=4600) and acetone was prepared by addition of 1600 mL of acetone to one 64-oz jar followed by addition of 16.0 grams of PEG 4600 and 144 grams of Example 5 polymer. The solution was enclosed and briefly heated to facilitate dissolution. The jar was placed on an automatic rolling apparatus until complete dissolution was reached. Continuous impregnation of the knitted core was performed by the continuous movement of a knitted core through a 0.75-liter bath of polymer solution. The knitted core was unspooled from the beginning of the impregnating apparatus (consisting of the spools; the coating bath; and the collection spool) and immediately fed into the bath of polymer solution, where two in-line submerged pulleys keep the scaffold materials submerged for the length of the bath. As the impregnated material exits the bath, it was passed through an air-circulating drying tube heated to 40° C., then a stainless steel element heated to 50° C., and then collected onto a final take-up spool, where it was stored under reduced pressure to complete the drying process.

Intravesical degradable medical devices and/or compositions were formed from the impregnated, knitted core by wrapping onto racks equipped with two parallel stainless steel bars of 0.5 inch diameter, which were adjustable for separation distance to control final intravesical device length. Once placed on the forming rack, the structure was annealed at 130° C. for 30 minutes and allowed to cool to room temperature in a laminar flow hood. The intravesical devices were cut from the forming rack into the final net shape of the Degradable Ureteral Intravesical device and the Teflon core removed by securing one end of the Teflon to position-fixed vice grip as the opposite end was used to stretch the Teflon core to reduce diameter allowing removal from the intravesical device length. Each intravesical device created in this way was inspected to the appropriate specification.

Through this technique of coating and shape forming, the longitudinal slit created along the length of the inner coil remains but was initially protected due to the impregnation process. This is an example of a slitting and coating technique that may be used in connection with a medical device and/or composition, e.g., any of the medical devices and/or compositions disclosed herein, to provide managed degradation when the device was implanted into a subject.

### Example 12

### In Vitro Hydrolysis of Intravesical Device Prepared with a Longitudinal Slitting of the Coil Component

The impregnated and shaped degradable intravesical device from Example 11 degraded primarily from hydrolysis of main chain esters of the polymer backbone. During in vitro hydrolysis of the impregnated and shaped degradable ureteral intravesical device from Example 11, the longitudinal slit created a defect that was predisposed to fracture at the slit site. Upon incubation in artificial urine at 37° C., the degradable intravesical device initially lost strength along the entire length of the intravesical device while maintaining bulk form for the first about 5-7 days (compared to about 7-12 days if the slit was absent). See ASTM F1828-97 (Reapproved 2006), preparation method A1.2 for the composition of the artificial urine.

After this time, degradation primarily within the glycolide containing monofilament coil reduced the tensile strength, approximately to less than about 20% of the initial strength, creating a friable monofilament that fractured with mechanical input. The coil initially fractured at the slit location prior to fragmenting from the degradable ureteral intravesical device. This fragment was of between 0.03 mm to 15 mm in length, which then readily fractured into smaller segment lengths with intravesical device movement. The degradable ureteral intravesical device as described in Example 11 hydrolysed into segment lengths suitable for passage from the urinogenital conduit within about 30 days of placement.

### Example 13

### Alternative Methods for Creating a Defect to Provide a Predictable Coil Failure Point

A defect that creates managed degradation of a biodegradable degradable medical device and/or composition was imparted in a number of ways. As previously exemplified, the coiled form of monofilament was cut to provide a slit of a specified depth. The cut depth was selected to have a desired impact on the fiber tensile strength, with increasing depth of cut directly correlated with increasing loss in tensile strength. Cut depths of more than 30% of the fiber diameter resulted in a tensile strength loss of 50% or more from the initial strength, yielding mechanical performance of the coil which may be below that needed to maintain ureter patency. Accordingly, in one aspect the cut depth was less than 30% of the diameter of the coiled monofilament. Increased cut depth provided faster degradation of the coil portion of the intravesical device, however this must be balanced against providing the strength retention time required for intravesical device functionality.

The cut need not be uniform. For example, shallow cuts of at least 0.03 mm were made in an interrupted manner along the axis of the coil as opposed to a continuous manner in which each individual coil has a cut. This served to create larger fragments of the intravesical device during the degradation process, whereas intravesical device fragments that are longer than 10 mm may not be desirable due to the potential for obstruction.

Instead of creating a cut along the coil with a knife-edge cutter, alternative techniques to disrupt the coil portion of the degradable medical device and/or composition were employed. For example, a laser was used to create a slit by ablating between 3-30% of the coil diameter from the intravesical device without creating a large heat affected zone.

The laser was also, or alternatively, used to provide a point energy source to create one or more regions of stress through disorganizing the polymer crystalline structure, imparting stresses within the coil polymer, and degrading the polymer chain length, all in order to elicit a more rapid degradation. A combination of heating and laser ablation was also used for a combined effect.

These are examples of creating defects in a medical device and/or composition, e.g., any of the medical devices and/or compositions disclosed herein, to provide managed degradation when the device was implanted into a subject.

### Example 14

### Creation of a Degradation Site Through the Selective Removal of Intravesical Device Impregnation Coating

An impregnated and shaped degradable ureteral intravesical device was modified to encourage degradation into smaller sections through the selective removal of the impregnation coating along the length of the intravesical device. The impregnated and formed intravesical device was processed by removing circumferential bands of coating spaced a distance part, e.g., 1 cm apart, along the shaft length, i.e., along the mid-section of the intravesical device. This was accomplished by applying a suitable solvent such as acetone to dissolve the impregnation coating in a narrow band of a desired width, for example, of approximately 1 mm in width, from the shaft, and around the entire circumference of the intravesical device. This process exposed the treated sections of knit and coil components of the intravesical device. The resulting intravesical device comprised a continuous coil, e.g., formed of MG5-B, a continuous knitted component encasing the coil, and an impregnation coating along the length of the intravesical device in sections of, e.g., 1 cm in length followed by spacing, e.g., a 1 mm separation, prior to the next section of impregnated coating.

This illustrates how to create a defect in a degradable medical device and/or composition which may be applied to any medical device and/or composition, including any of the medical devices and/or compositions disclosed herein, to provide managed degradation when the device was implanted into a subject.

### Example 15

### In Vitro Hydrolysis of the Degradable Intravesical Device with Selectively Removed Sections of Impregnation Coating

The impregnation coating of a degradable intravesical device acts essentially as a passivation layer to control the rate of hydrolysis of the underlying coil component as well as the knitted component. During hydrolysis of the intravesical device created in Example 14, sections with coating removed were less protected from degradation. In vitro hydrolysis of the intravesical device from Example 14 was performed using simulated urine at 37° C. Sections of the intravesical device with the impregnation coating selectively removed were expected to lose strength approximately 20% faster than those with coating, encouraging fractures to occur at those sites thereby generating degraded lengths of approximately 1 cm to allow easier clearance from the ureter and bladder without obstruction.

### Example 16

### Method of Creating a Preferred Degradation Vector Through the Application of Ionizing Radiation

The application of ionizing radiation may be used to reduce the molecular weight of synthetic bioresorbable polyesters. In an effort to generate a preferred degradation vector or gradient in a intravesical device, beta radiation may be applied from one direction along the length of the intravesical device with a total exposure of the bladder curl side of the intravesical device of about 10-50 kGy, transitioning to about a 50% reduced irradiation dose at the kidney curl side of the intravesical device.

To generate this dose vector, the intravesical device as described in Example 6 is placed in a metal foil pouch and dried under reduced pressure at room temperature to minimize residual moisture. The packaged is then hermetically sealed in a nitrogen atmosphere to provide protection from light and moisture. The sealed packages containing the intravesical device were packaged with the bladder curl end upright in a single layer for processing.

Electron irradiation, a process involving beta radiation of high energy is applied to the packaged intravesical device. In this technique, a cathode source is used to generate electrons that were accelerated and shaped into a collimated beam. This process produces radiation which is limited in the depth of penetration. By applying this technique to a box containing intravesical devices where the intravesical devices were oriented such that the bladder curl is closest to the radiation source, the bladder curl receives an elevated dose of radiation, with effective applied dosage lessening through the length of the packaging, ultimately with the kidney curl end of the intravesical device obtaining approximately 60% of the radiation energy of the bladder curl end.

By creating an intravesical device with progressive radiation dose, the degradation profile is modulated such that the intravesical device preferentially fractures starting at the bladder curl progressing towards the kidney curl, thereby promoting small size degradation products which minimize risk of intravesical device-induced incontinence.

The present disclosure thus provides a process for producing a degradable medical device and/or composition wherein electron radiation is applied in a non-uniform manner across the device, so that the device itself comprises a polymer having a non-uniform molecular weight (M_{w} or Mₙ) across the device. The present disclosure also provides devices that have a gradually changing non-uninform polymer molecular weight (M_{w} or Mₙ) across a dimension of the device.

This illustrates how to create a defect in a specific degradable medical device and/or composition where this approach may be applied to any medical device and/or composition, including any of the medical devices and/or compositions disclosed herein, to provide managed degradation when the device is implanted into a subject.

### Example 17

### Method of Creating a Preferred Degradation Vector Through the Modulation of Coil Pattern & Coil Density

A method for shape retention during degradation is the presence of the coil within an intravesical degradable medical device and/or composition. In order to create a preferential degradation path, in one aspect the intravesical device coil is modulated along the length of the intravesical device shaft during coil manufacturing. To achieve this result, a Teflon monofilament acts as a core to create and maintain the inner lumen of the intravesical device during manufacturing. Example 1 polymer or other monofilament fiber is wrapped around the Teflon core to create the coil component of the intravesical device. During coil creation, sections of the coil that will ultimately become the bladder curl and kidney curl were formed at a coil density of about 33 to 35 coils per inch. In the shaft region of the intravesical device, which forms the mid-section of the intravesical device, the coil is created with a modulated coil density progressing from about 33 to 35 coils per inch at the kidney curl transition to about 15 to 20 coils per inch at the bladder curl transition.

The coil densities may be varied at the beginning, the end, and along the shaft. For example, the shaft region of the intravesical device may have a coil density at one of the shaft (e.g., near the kidney curl transition) of, for example 30, or 31, or 32, or 33, or 35 coils/inch. The coil density may gradually decrease along the length of the shaft. For example, at the bladder coil transition the coil density may only be, for example, 12, or 13, or 14, or 15, or 16, or 17, or 18, or 19, or 20, or 21, or 22, or 23, or 24, or 25 coils/inch.

During in vitro degradation at 37° C. in simulated urine, the section of the intravesical device shaft with 15-20 coils per inch loses strength to a level that supports intravesical device fracture several days, e.g., 1, or 2, or 3 or 4 or 5 days earlier than the transition closest to the kidney, providing a bladder-to-kidney degradation vector. As the intravesical device shaft progressively begins to fracture, small sections were separated from the intravesical device shaft providing a “bottom-up” degradation path and minimizing the risk of intravesical device-induced incontinence.

This illustrates how to create a degradation vector in a specific degradable medical device and/or composition where this approach may be applied to any medical device and/or composition, including any of the medical devices and/or compositions disclosed herein, to provide managed degradation when the device is implanted into a subject.

### Example 18

### Alternative Methods of Creating a Preferred Degradation Vector Through the Modulation of Intravesical Device Components

A degradation vector was created along the intravesical device shaft. The impregnation coating was applied to a coil, e.g., glycolide polymer based coil, on a Teflon core through a spray coating technique, where the deposited thickness was varied along the length of the shaft. According to this approach, the impregnation coating thickness was applied at a level of 8 wt % of the total weight of coatings on both curls as well as at the kidney curl transition, while the coating was applied at a level of 4 wt % at the bladder curl transition, with a linear coating gradient between the bladder and kidney curl. By this approach, the integrity of the intravesical device was lost first at the bladder end of the shaft.

In other aspects, the impregnation coating thickness was applied at a level of 5 wt %, or 6 wt %, or 7 wt %, or 8 wt %, or 9 wt %, or 10 wt %, or 11 wt %, or 12 wt % of the total weight of coatings on both curls as well as at the kidney curl transition, while the coating was applied at a lower level at the bladder curl transition, with a linear coating gradient between the bladder and kidney curl. For example, if the impregnation coating thickness was at a maximum of 12 wt %, the coating thickness may gradually decrease to 11 wt %, then 10 wt %, then 9 wt %, then 8 wt %, etc. down to the desired thickness, thus creating a coating thickness gradient.

These examples illustrate how to create a degradation vector in a specific degradable medical device and/or composition where these approaches may be applied to any medical device and/or composition, including any of the medical devices and/or compositions disclosed herein, to provide managed degradation when the device was implanted into a subject.

### Example 19

### Alternative Methods of Creating a Degradation Vector Through the Modulation of Intravesical Device Components

Another way to create a preferred degradation vector for a degradable intravesical device having a coil, knit, and coating construct as described earlier was to plasma treat the surface of the device. Plasma treatment added hydroxyl groups to the surface of the device, which recruited more water when the device was degradable medical device and/or composition in a subject, and thereby encouraged degradation at the surface. The plasma treatment was selective so that some surfaces received more plasma treatment, and accordingly had more hydroxyl groups, than other surfaces. In this way, a gradient of hydroxyl groups was created along the intravesical device shaft with the highest level of exposure at the bladder curl transition to increase local hydrophilicity and locally degrade/disorganize the polymers, thereby encouraging initial strength loss to occur closest to the bladder curl, progressing along the intravesical device shaft towards the kidney curl.

This chemical treatment to induce early degradation was applied through alternative means as well, including acid treatment at varying levels to induce chain scission and hydrophilicity. Additionally, the spray coating composition was modulated along the length of the intravesical device by changing the ratio of PEG 4,600 to SVG12, with a higher concentration of PEG 4,600 at the bladder curl transition and lower concentration at the kidney curl transition.

This illustrates how to create a degradation vector in a specific degradable medical device and/or composition where this approach may be applied to any medical device and/or composition, including any of the medical devices and/or compositions disclosed herein, to provide managed degradation when the device was implanted into a subject. Thus, the present disclosure provides a method of forming a degradable intravesical device, the method comprising constructing a degradable intravesical device from components comprising a coil, a knit, and a coating, and then subjecting the intravesical device to chemical treatment to induce early degradation of the intravesical device when it was implanted into a subject.

### Example 20

### Design and Manufacturing to Create Layered Core-to-Sheath Degradation Vector

A degradable ureter intravesical device was made having an innermost coil made from a radiopaque, glycolide-based monofilament, overknit with a glycolide-based multifilament, and subsequently sheathed with a degradable polymeric film, where the monofilament coil exhibits the fastest degradation profile, followed shortly by the overknit multilament, and lastly the film sheath exhibiting the longest degradation life.

Monofilament coil of 0.3 mm diameter, described above, was wound onto a Teflon core having a 0.055″ diameter at between 33 and 35 coils per inch. A Lamb circular weft knitter was used to form a knit of Glycoprene multifilament fiber over the coiled and slitted scaffold described previously in a continuous manner. A ⅞″ knitting cylinder with 12 course gauge needles was used to form the knitted scaffold over the coiled scaffold. A ¾″ single barrel custom melt extruder with a tubing die heated to approximately 165° C. was used to extrude SVG-12 and polyethylene glycol blend into a thin sheath. The coiled and knitted Teflon assembly was passed through the tubing die head allowing direct application of the thin tubular sheath.

Intravesical devices were formed from the impregnated, knitted core by wrapping onto racks equipped with two parallel stainless steel bars of 0.5 inch diameter, which were adjustable for separation distance to control final intravesical device length. Once placed on the forming rack, the structure was annealed at 130° C. for 30 minutes and allowed to cool to room temperature in a laminar flow hood. The intravesical devices were cut from the forming rack into the final net shape of the degradable ureter intravesical device and the Teflon core removed by securing one end of the Teflon to position-fixed vice grip as the opposite end was used to stretch the Teflon core to reduce diameter allowing removal from the intravesical device length. Each intravesical device created in this way was inspected to the appropriate specification. In this manner, a degradable ureter intravesical device was prepared such that the sheath remained as a distinct layer which maintained the intravesical device outer diameter but did not individually coat the coil or Glycoprene (see Example 5) knit.

During in vitro degradation at 37° C. in simulated urine, the coil lost strength at between about 7 and 14 days, after which it fragmented and was easily displaced from the remaining Glycoprene fiber and outer sheath. As the coil, and later the knit, fragmented they separated from the residual intravesical device and settle in the bladder, further degrading to smaller fragments before excretion with simulated urination. Likewise, the sheath may collect in the bladder and fragments before excretion with simulated urination.

This illustrates how to create a degradation vector in a specific component of a degradable medical device and/or composition where this approach may be applied to any medical device and/or composition, including any of the medical devices and/or compositions disclosed herein, to provide managed degradation when the device was implanted into a subject.

### Example 21

### Multiple Coil Inclusion to Control Rate of Coil Separation

Polydioxanone polymer is melt-extruded using a ¾″ single screw extruder into a monofilament, followed by orientation and heat treatment to create a thermally stable, strong monofilament with 0.3 mm diameter. Glycolide stabilized monofilament fiber is coiled onto a monofilament Teflon core with 0.055″ diameter in a continuous manner using a Lamb circular knitter. In alternating sections of 15 cm, the polydioxanone monofilament is co-coiled with the glycolide monofilament to impart a second coil component. Throughout both sections of coil (with and without polydioxanone monofilament), the net coil density remains constant at between 33 and 35 coils per inch. The resulting coil is processed into a finished degradable intravesical device as described in Example 6 to form a intravesical device with Example 1 polymer only coil from roughly the midpoint of the shaft through the bladder curl. The portion of the intravesical device between the shaft midpoint and the tip of the kidney curl contains coil of both glycolide-based and polydioxanone monofilaments.

During in vitro degradation at 37° C. in simulated urine, the section of the intravesical device below the midpoint of the shaft will fracture and separate from the intravesical device body first. The Example 1 polymer coil within the kidney half of the intravesical device will be retained within the intravesical device due to the extended strength retention of the second coil component. After 14 days, when the Example 1 polymer is significantly degraded and fragmented, the polydioxanone coil begins to fragment, releasing fine particulate Example 1 polymer, the residual coating and knit components, ultimately allowing the residual polydioxanone coil to transfer into the bladder. These fragments are further degraded within the bladder until excreted through simulated urination.

Thus, the present disclosure provides degradable medical devices and/or compositions comprising two different polymers, each having a unique degradation profile (i.e., the two polymers have different degradation profiles, one from another), where the two polymers are each used to prepare the same component of the medical device and/or composition. In this example, that same component is the coil of a intravesical device. Thus, the coil component of the intravesical device is made from two coils, where the two coils comprise different polymers having different degradation profiles.

### Example 22

### Manufacturing Alternatives to Generate Discrete Degradation Particulate: Modulated Monofilament Diameter

The coil component of a degradable ureter intravesical degradable medical device and/or composition is typically the primary feature for providing structural support for the intravesical device, as well as the primary strength mechanism for maintaining lumen patency once implanted. To encourage the monofilament to degrade into discrete particulates, the coil material, e.g., MG5-B, is melt-extruded into a continuous monofilament and oriented with between 2× and 5.5× draw ratio in a pulsatile manner with a period of 5 mm so that the resulting fiber exhibits a diameter of 0.3 mm at the crest and 0.2 mm at the trough. These diameter values are exemplary only: other non-identical diameter values may also be created using the pulsatile manner of extrusion. This monofilament is subsequently processed by wrapping onto a Teflon core having 0.055″ diameter at a coil density of between 33 and 35 coils per inch. This coil is additionally processed as described in Example 6 to form a degradable ureter intravesical device.

During in vitro degradation at 37° C. in simulated urine, the Example 1 polymer monofilament with variable diameter preferentially degrades at the smallest diameter sections of the monofilament, generating Example 1 polymer particulates which are approximately 5 mm in length.

This illustrates how to create a non-homogeneity in a specific degradable medical device and/or composition where this approach may be applied to any degradable medical device and/or composition having a filament, including any of the medical devices and/or compositions disclosed herein that include a filament, to provide managed degradation when the device is implanted into a subject.

### Example 23

### Manufacturing Alternatives to Generate Discrete Degradation Particulate: Embossed Monofilament

Example 1 polymer monofilament is melt-extruded and oriented into a uniform diameter. In a secondary process, the monofilament is passed through a rotary embosser with a dimple pattern at 2-10 mm increments to generate stress concentration and physical defects along the length of the fiber. Embossed monofilament is then wrapped onto a Teflon core having 0.055″ diameter at a coil density of between 33 and 35 coils per inch and further processed as described in Example 6 to form a degradable ureter intravesical device.

During in vitro degradation at 37° C. in simulated urine, the embossed monofilament preferentially degrades at the location of the embossed dimple, generating Example 1 polymer particulates which are approximately 5 mm in length.

This illustrates how to create a defect in a specific degradable medical device and/or composition where this approach may be applied to any medical device and/or composition, including any of the medical devices and/or compositions disclosed herein, to provide managed degradation when the device is implanted into a subject.

### Example 24

### Manufacturing Methods to Generate Discrete Degradation Particulate: Heat Treatment

Example 1 polymer monofilament is melt-extruded and oriented into a uniform diameter. In a secondary process, the monofilament is partially thermally treated in 2 mm segments along the fiber axis, reducing the crystalline orientation of the Example 1 polymer monofilament. Partially heat treated monofilament is then wrapped onto a Teflon core having 0.055″ diameter at a coil density of between 33 and 35 coils per inch and further processed as described in Example 6 to form a degradable ureter intravesical device.

During in vitro degradation at 37° C. in simulated urine, the partially heat treated monofilament preferentially degrades at the location of thermal treatment, generating Example 1 polymer particulate which is approximately 2 mm in length.

This illustrates how to create a defect in a specific degradable medical device and/or composition where this approach may be applied to any medical device and/or composition, including any of the medical devices and/or compositions disclosed herein, to provide managed degradation when the device is implanted into a subject.

### Example 25

### Manufacturing Alternatives to Generate Discrete Degradation Particulate: Ringed Support

Example 1 polymer is injection molded into an open ring structure (clip) having an inner lumen diameter of 0.050″ and a wall thickness of 0.3 mm. The coil is further designed such that the “top” and “bottom” surface of the clip have flat features for improved stacking and handling during downstream processing and to aid in buckling resistance during intravesical device insertion. These clips are placed on a Teflon core with 0.055″ diameter at a spacing of between 30 and 35 clips per inch and remain in place by frictional forces. The Teflon core with Example 1 polymer clips are further processed as described in Example 6 to form a degradable ureter intravesical device.

During in vitro degradation at 37° C. in simulated urine, the injection molded clips separate due to degradation in the knit and coating layers, generating discrete particulates which are less than 0.3 mm in thickness.

This illustrates how to create a defect in a specific degradable medical device and/or composition where this approach may be applied to any medical device and/or composition, including any of the medical devices and/or compositions disclosed herein, to provide managed degradation when the device implanted into a subject.

### Example 26

### Manufacturing Alternatives to Generate Discrete Degradation Particulate: Islands in the Sea

Example 1 polymer and 85:15 PLGA are coextruded into an islands-in-the-sea monofilament configuration, with between 1 and 10 filaments of 85:15 PLGA at 5 denier per filament comprising the “islands” within a “sea” of MG5-B. The monofilament is oriented to increase tensile strength and heat stabilized to optimize thermal stability. This attenuated monofilament is wound around a Teflon core having a 0.055″ diameter at a spacing of between 33-35 coils per inch. The coiled Teflon core is further processed as described in Example 6 to form a degradable ureter intravesical device.

During in vitro degradation at 37° C. in simulated urine, the Example 1 polymer component of the coil loses strength before the 85:15 PLGA “islands”, which assists in retaining the initial coil structure of the intravesical device until the Example 1 polymer component is degraded such that it fragments into pieces smaller than 0.5 mm. Once the MG5-B component of the coil is separated from the “islands,” the filaments generally coalesce into a small, loosely condensed structure allowing easy excretion from the bladder with urination.

This illustrates how to create a defect in a specific degradable medical device and/or composition where this approach may be applied to any medical device and/or composition, including any of the medical devices and/or compositions disclosed herein, to provide managed degradation when the device is implanted into a subject.

### Example 27

### Manufacturing Alternatives to Generate Discrete Degradation Particulate: Monofilament Shape

Example 1 polymer is melt extruded using a single screw ¾″ extruder with a multi-lobed die having a plurality of fins from a central hub, to create a profile monofilament with a cross sectional area of approximately 0.07 mm². After orientation and heat treatment, the monofilament is coiled around a 0.055″ Teflon core at a density of between 33 and 35 coils per inch to form a intravesical device coil.

This illustrates how to create an asymmetric component in a specific degradable medical device and/or composition where this approach may be applied to any medical device and/or composition, including any of the medical devices and/or compositions disclosed herein, to provide managed degradation when the device is implanted into a subject.

### Example 28

### Alternative Monofilament Shapes

Example 1 polymer may be melt extruded into a variety of non-circular cross-sectional shapes to control degradation morphology. For example, the polymer may be melt extruded into the form of a bi-lobed monofilament with a narrow central section to induce degradation primarily into two separate longitudinal lengths to increase compliance of the degradation byproduct. Alternatively, the polymer may be melt extruded into the form of a flattened monofilament or a tube to maintain bending stiffness and to maintain intravesical device mechanics while encouraging the degradation of the coil component into smaller particulates.

This illustrates how to create an asymmetric component in a specific degradable medical device and/or composition where this approach may be applied to any medical device and/or composition, including any of the medical devices and/or compositions disclosed herein, to provide managed degradation when the device is implanted into a subject.

### Example 29

### Inclusion of Buffering Agent to Counteract Degradation Byproduct Acidity

Degradable ureter intravesical devices are prepared as described in Example 6, with a modified coating solution. The coating solution is prepared using SVG-12, polyethylene glycol (Mw=4,600), and micronized dibasic sodium phosphate (e.g., at 5 wt %), dissolved and dispersed into acetone. The resulting degradable ureter intravesical device impregnation coating contains approximately 0.5-3 wt % of dibasic sodium phosphate. After implantation, the dibasic sodium phosphate is released from the coating and assists to buffer the urine against the effects of acidic degradation byproducts to maintain a typical pH level within the urine of between 6.5 and 8.0.

### Example 30

### Inclusion of Antispasmodic to Counteract Irritation Induced by Foreign Object

Degradable ureter intravesical devices are prepared as described in Example 6, with a modified coating solution. The coating solution is prepared using SVG-12, polyethylene glycol (Mw=4,600), and an antispasmodic agent such as Atropine, dissolved and dispersed into acetone. The resulting degradable ureter intravesical device impregnation coating contains at least 0.5 mg of Atropine. After implantation, the Atropine is released from the impregnation coating and acts with anticholinergic action to control spasms within the ureter and urethra which may be caused by the presence of a foreign body or breakdown product therefrom.

### Example 31

### Intravesical Device Surface Feature to Improve Placement Stability

A degradable ureter intravesical device is prepared as described in Example 6 but using a barbed monofilament fiber with 0.3 mm diameter as the coil component. The resulting intravesical device exhibits barbed surface features which act to penetrate into the tissue after placement to reduce the risk of intravesical device migration. Optionally, the intravesical device is produced without a bladder curl as placement location stability is maintained by the presence of the barbs on the intravesical device surface. Optionally, the bladder curl is replaced with a flared section to prevent upward migration of the intravesical device into the kidney.

### Example 32

### Additive Manufacturing Approach to Form a Degradable Ureter Intravesical Device

To create a continually variable degradation path with includes surface features for placement stability, an additive manufacturing approach is utilized. A Stratasys J750 polyjet printer is utilized to deposit three distinct materials to provide component elements of the final intravesical device structure, as described:

Component 1 is a fast-degrading glycolide-based photocurable resin loaded with an inorganic radiopacifier, and is used as a radial stiffening element and printed within the body of the intravesical device as a coil.

Component 2 is a fast-degrading poly(propylene fumarate) photocurable resin which is included axially throughout the shaft of the intravesical device to act as a reinforcing element for placement and potential retrieval.

Component 3 is a flexible trimethylene carbonate-based photocurable resin and is included as a tissue-interfacing covering to form the sheath of the intravesical device and bind components 1 and 2, and also to maintain the net intravesical device shape.

Component 1 is included within the intravesical device such that the orientation and shape of the coil structure provides a flat inner lumen, as well as an outer surface that is textured to increase friction between the ureter wall and the intravesical device surface for improved placement stability.

By modulation of Component 3 thickness within the intravesical device it is possible to provide for faster degradation in specific locations to create defects that fracture at between 1 and 3 days faster than those with more Component 3 covering. Additionally, Component 3 is designed to optionally create a porous (non-continuous) film barrier.

Component 2 is included to maintain continuity of the intravesical device, and is included as an axial component in an interrupted manner to allow for fracture and separation of the intravesical device fragments at specific sites.

The net intravesical device shape, including both bladder curl and kidney curl, are generated in a single process with no need for additional curing or heat treatment. The intravesical device is cleaned in an isopropanol bath at 37° C. for 1 hour to remove residual photoinitiator and unreacted reagents before drying, packaging and sterilization.

During in vitro degradation at 37° C. in simulated urine, fragmentation preferentially occurs in regions with less Component 3. The connectivity of the intravesical device is maintained by Component 2, whereas when the coil fractures intravesical device unity is maintained within the intravesical device structure until coil fracture occurs at an interruption in Component 2. This fracture allows a small section of the intravesical device to break away into the bladder, where it may be excreted with urination.

### Example 33

### Coil Component Modulus

USD polymer is prepared by ring-opening polymerization with polydioxanone and a PEG macromonomer (Mw=10,000) to form a semi-crystalline polymer having melting temperature of between 100-120° C. The USD polymer is isolated, ground, and sieved to obtain a granule size of between 1-4 mm, and devolitalized to remove residual monomer.

USD polymer is melt-extruded utilizing a ¾″ single screw extruder into a monofilament. The monofilament is oriented at between 2× and 5.5× and annealed at 80° C. to increase thermal stability. Fiber tensile testing identifies a tensile modulus of between about 500-800 MPa, which is approximately 2 times that of Example 1 polymer monofilament. USD monofilament is coiled around a Teflon core of 0.055″ with a coil density of 30-40 coils/inch, or a coil density between 33 and 35 coils per inch. The coiled monofilament is processed into a degradable ureter intravesical device in the same manner as described in Example 6.

During in vitro degradation at 37° C. in simulated urine, the polyether component of the USD monofilament coil leads to degradation and fragmentation of the coil between about 10 and 21 days, allowing the residual intravesical device fragments to transfer into the bladder. Due to the increased modulus of the USD monofilament compared to Example 1 polymer monofilament, the coil fragments tend to retain the coiled shape and prevent other intravesical device components from collapsing and collating into a dense mass, which may help reduce the risk of incontinence and/or blockage within the urethra.

### Example 34 Inherently Radiopaque Polymer

A coating polymer, RS-1, is synthesized using 33% ε-caprolactone, 32% l-lactide, 17% glycolide, 14% trimethylene carbonate, with 4% 3-iodo-1-propanol as initiator. Stannous octoate is used as catalyst. The polymer is isolated, dissolved in dichloromethane, and precipitated into cold isopropanol to remove impurities, then dried before analysis and storage.

Degradable ureter intravesical devices are produced by first creating a coiled construct with Example 1 polymer, without the radiopaque additive. Glycoprene multifilament fiber is weft knitted over the coil component. RS-1 is dissolved in acetone and the coil/knit components are coated via continuous dip coating to create a coated scaffold. The coated scaffold is formed into the intravesical device net shape as described in Example 6.

The coating prepared from RS-1, as a result of iodine content within the polymer, is inherently radiopaque, creating a intravesical device that can be visualized by x-ray after implantation. As opposed to other disclosed intravesical device structures described in earlier examples which contain solid inorganic particulate as a radiopacifier, the intravesical device in this example contains no solid inorganic microparticulate. As the intravesical device degrades in vivo, the absence of inorganic microparticulates minimizes the inflammatory response resulting from high hardness microparticulates.

### Example 35

### Gel Former Formulation 1

Gel-based Formulation 1 was prepared using 2 parts of a poly ester-ether-urethane block copolymer comprising a 70/17/13 poly(d,l-lactide-co-glycolide-co-polyethylene glycol) block copolymer interlinked with 1, 6-hexamethylene diisocyanate (OC9) with 1 part PEG400 (Sigma Aldrich) as diluent. The mixture was combined using a centrifugal mixer until uniform. Erdafitinib was loaded into the formulation at 40 mg/g of OC9 and mixed using a centrifugal mixer until uniform, creating a uniformly yellow thick liquid.

### Example 36

### Gel Former Formulation 2

Gel-based Formulation 2 was prepared using OC9 (a poly ester-ether-urethane block copolymer comprising a 70/17/13 poly(d,l-lactide-co-glycolide-co-polyethylene glycol) block copolymer interlinked with 1, 6-hexamethylene diisocyanate) and acetone as a diluent. When OC9 was completely dissolved, Erdafinitb was loaded into the solution at 40 mg/g of OC9 and mixed using a centrifugal mixer until uniform. This solution was poured onto a Teflon sheet and placed under vacuum to remove acetone, forming a thick and uniformly yellow liquid.

### Example 37

### Encapsulated Formulation 3

SVG12 (35/34/17/14 poly(caprolactone-co-lactide-co glycolide-co-trimethylene carbonate) (Poly-Med, Inc., Anderson, S.C.) was used without further modification. A 15% solution was prepared using dichloromethane as solvent, which was cast into a thin film using a 15 mil film-casting blade and allowed to dry. The final film was 0.03 mm thick.

A portion of Formulation 2 (about 0.3 g) was formed into a soft ball and added to the center of the SVG12 film, and the film folded over to cover the soft ball A sealing line was created by solution welding the perimeter of the film, thereby encapsulating the gel formulation.

### Example 38

### Encapsulated Formulation 4

A thin film was prepared using SVG12 as described in Example 37, with the addition of PEG400 (Sigma Aldrich) to the solution at a ratio of 4:1 SVG12:PEG400. The solution was cast into a thin film using a 15 mil film-casting blade and allowed to dry. The final film was 0.03 mm thick.

A portion of Formulation 2 (about 0.3 g) was formed into a soft ball and added to the center of the SVG12 film, and the film folded over to cover the soft ball. A sealing line was created by solution welding the perimeter of the film, thereby encapsulating the gel formulation.

### Example 39

### Erdafitinib Release Study

Triplicate samples from Examples 35-38 (summarized in Table 1) were obtained and covered in artificial urine at 37° C. to perform an elution study. Artificial urine was prepared according to ASTM standard F1828-97.

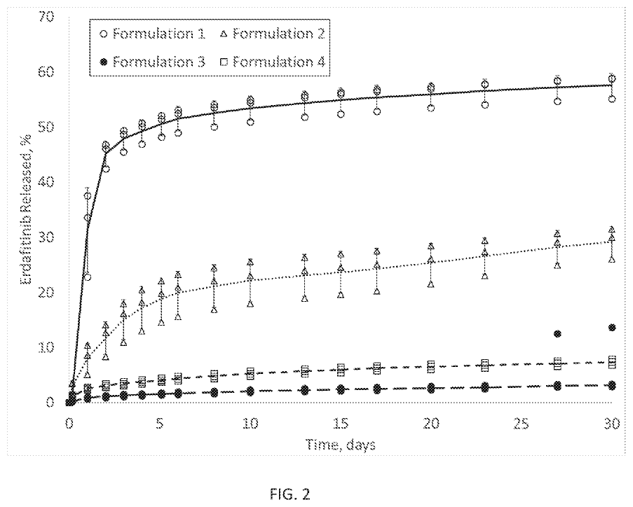

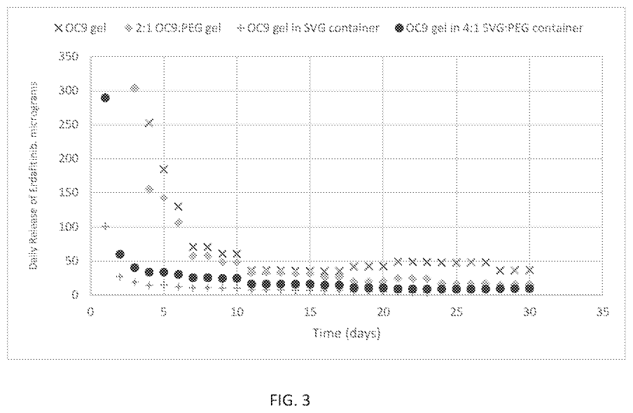

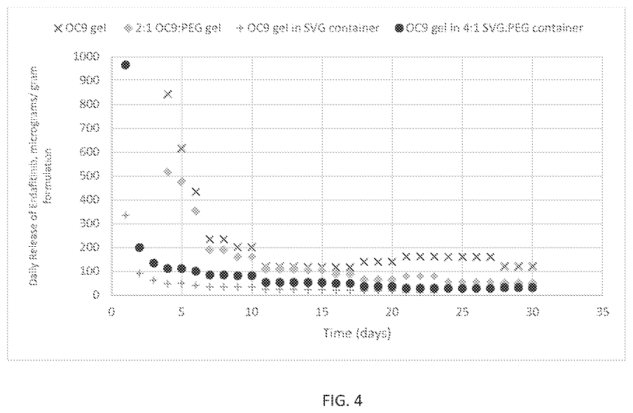

At each time point, the artificial urine was completely changed and fresh artificial urine added. Samples were analyzed by HPLC (Waters) equipped with a C18 column at 254 nm, using an isocratic method at 70% Solvent A (water with 0.1% TFA) and 30% Solvent B (acetonitrile with 0.1% TFA). Peak areas were compared against a standard curve to obtain concentrations, and the percent Erdafitinib release was calculated, with results provided in FIGS. 2, 3 and 4. The data showed that the amount of Erdafitinib, as a percentage of the initial loaded amount, and the rate at which the Erdafitinib was released can be altered by changing the matrix used to release the active agent.

TABLE 1
Formulation Summary

| | | | | Containment |
|---|---|---|---|---|
| | | | | Layer |
| Formu- | | Erdafitinib | Containment | Thickness |
| lation | Gel Matrix | (mg/g OC9) | Layer | (mm) |
| 1 | 2:1 | 40 | — | — |
| | OC9:PEG400 | | | |
| 2 | OC9 | 40 | — | — |
| 3 | OC9 | 40 | SVG12 | 0.03 |
| 4 | OC9 | 40 | 4:1 | 0.03 |
| | | | SVG12:PEG400 | |

### Example 40

### Triamcinolone- and Lidocaine-Eluting Encapsulated Gel Films for Bladder Delivery

OC9 (Poly-Med, Inc.) was used as a gel-forming, urethane-linked degradable copolymer comprising repeat units of 70% d,l-lactide, 17% polyethylene glycol, and 13% glycolide. First, OC9 was heated at 50° C. for 3 hours to improve miscibility and blended with PEG 400 to reduce gel viscosity. Triamcinolone acetonide (98+%, Alfa Aesar) was added and mixed with a dual-axis high speed rotary mixer to uniformly incorporate. Triamcinolone-loaded OC9 was transferred into a syringe to aid in part formation. At the same time, SVG12 (Poly-Med, Inc.) was admixed in dichloromethane at 15% concentration. Lidocaine powder (Sigma Aldrich) was added to the solution at a concentration of 134 mg per gram of SVG12 and dissolved. The solution was cast into a thin film using a 25-mil film casting blade and allowed to dry. A portion of the triamcinolone-loaded gel was added on top of the lidocaine-loaded SVG12 film, and the film was folded over to cover the gel. The edges of the device were sealed by solution welding the perimeter, thereby encapsulating the gel formulation.

TABLE 2
Triamcinolone- and Lidocaine- Eluting Encapsulated Gel Films
Prepared for Bladder Delivery and Localized Via Size

| | | Drug | | |
|---|---|---|---|---|
| | Active Agent, | containing | Encapsulation | Insertion |
| Form | % Loading | Polymer | Polymer | Method |
| 3 cm × 4 cm | Triamcinolone, | OC9, | SVG12 | Introduced |
| rectangle, | 10 mg/g (in | PEG 400 | | via 16F |
| 0.4 mm | core)Lidocaine, | | | Ureteroscope |
| thickness | 134 mg/g (in | | | with pusher |
| | encapsulation | | | |
| | polymer | | | |

By incorporating lidocaine in the encapsulation polymer layer, a relatively fast release rate can be achieved. For example, this may occur over the course of between 1 hour to 6 hours, or up to 12 hours, or up to 24 hours. This release duration can be influenced by encapsulation layer thickness and polymer properties, and further modified via additives such as polyethylene glycol or acid-terminated polyglycolide microdispersions.

Triamcinolone is a corticosteroid and incorporated for long term, low dose local therapy of interstitial cystitis and bladder pain. The installation is effective at providing delivery of triamcinolone over the course of 1 week to 4 weeks, and up to 8 weeks, for example. The longevity of the implant is based on the mechanical competency of the encapsulation layer, and upon mechanical degradation the implant is self-eliminated from the bladder with urination. Alternatively, for a time before self-elimination, the implant may be retrieved via minimally invasive surgical means.

### Example 41

### Drug Eluting Fabrics Prepared for Bladder Delivery and which are Capable of Positioning in a Subject Via Suture Attachment

SVG12 (Poly-Med, Inc.) and PDLG (Purasorb PDLG 7507, Corbion, a copolymer with 75:25 molar ratio of d,l-lactide and glycolide) were each dissolved in chloroform, each at 15% concentration. Solutions were divided to create different drug-loading solutions according to the associated table. In short, lidocaine (Sigma Aldrich) was added to a solution of SVG12 at a concentration of 134 mg drug per 1 gram of SVG12. Dexamethasone was added to a separate solution of SVG12 at a concentration of 29 mg drug per 1 gram of SVG12. Dexamethasone was added to a separate solution of PDLG at a concentration of 29 mg drug per 1 gram of PDLG. All solutions were held at room temperature on a roller until completely dissolved.

Fabrics were in the form of flat non-woven of various types to create differences in pore size, density, fiber surface area, and area weight (gram per square meter, gsm). Electrospun PDO (consisting of polydioxanone electrospun fabric) was used as an example fabric with ultrafine fibers and smaller pore size. Melt blown RD7 (consisting of a copolymer of glycolide, trimethylene carbonate, and caprolactone) has an intermediate fiber and pore size. Nonwoven chitosan (a naturally-derived polysaccharide in the form of felted staple fibers) has a larger fiber and pore size.

Fabrics were cleaned with isopropyl alcohol, air dried, and cut to approximately 4 cm×4 cm samples. Approximately 1001 μL of the respective solutions were applied to fabrics as indicated and allowed to air dry. Solutions effectively penetrated and wetted the fabric, thereby depositing the drug/polymer films within the void space of the fabric. Following an initial air dry, samples were placed under reduced pressure to remove residual solvent. Optionally, the drug-loaded polymer may be applied to select areas of the fabric.

TABLE 3
Example Drug Eluting Fabrics Prepared for Bladder Delivery and which
are capable of positioning in a subject via suture attachment

| | | Drug | | |
|---|---|---|---|---|
| | Active Agent, | containing | | Insertion |
| Form | % Loading | Polymer | Base Fabric | Method |
| 3.5 × 4 cm, | Lidocaine, | SVG12 | Electrospun PDO, | Introduced |
| 0.4 mm | 134 mg/g | | 55 gsm | via 16F |
| thickness | | | | Ureteroscope |
| | | | | with pusher |
| 3.5 × 3.5 cm, | Dexamethasone, | SVG12 | Electrospun PDO, | Introduced |
| 0.4 mm | 29 mg/g | | 55 gsm | via 16F |
| thickness | | | | Ureteroscope |
| | | | | with pusher |
| 4 × 4 cm, | Dexamethasone, | PDLG | Electrospun PDO, | Introduced |
| 0.4 mm | 29 mg/g | | 55 gsm | via 16F |
| thickness | | | | Ureteroscope |
| | | | | with pusher |
| 4 × 4 cm, | Dexamethasone, | SVG12 | Nonwoven Chitosan, | Introduced |
| 1 mm | 29 mg/g | | 113 gsm | via 16F |
| thickness | | | | Ureteroscope |
| | | | | with pusher |
| 4 × 4 cm, | Lidocaine, | SVG12 | Nonwoven Chitosan, | Introduced |
| 1 mm | 134 mg/g | | 113 gsm | via 16F |
| thickness | | | | Ureteroscope |
| | | | | with pusher |
| 4 × 4 cm, | Dexamethasone, | PDLG | Nonwoven Chitosan, | Introduced |
| 1 mm | 29 mg/g | | 113 gsm | via 16F |
| thickness | | | | Ureteroscope |
| | | | | with pusher |
| 4 × 4 cm, | Dexamethasone, | SVG12 | Meltblown RD7, | Introduced |
| 0.3 mm | 29 mg/g | | 105 gsm | via 16F |
| thickness | | | | Ureteroscope |
| | | | | with pusher |
| 4 × 4 cm, | Lidocaine, | SVG12 | Meltblown RD7, | Introduced |
| 0.3 mm | 134 mg/g | | 105 gsm | via 16F |
| thickness | | | | Ureteroscope |
| | | | | with pusher |

By incorporating lidocaine or dexamethasone in a polymeric coating, added to a nonwoven fabric, an implant was achieved having tensile strength and mechanical degradation rate similar to the base fabric. A relatively fast release rate can be achieved from an SVG12 polymer. For example, lidocaine may be released over the course of between 1 hour to 6 hours, or up to 12 hours, or up to 24 hours. PDLG polymer provides a longer release profile. For example, dexamethasone may be released over 1 day to 7 days, or up to 2 weeks, or up to 3 weeks. Release duration can be influenced by the polymer thickness, polymer properties, drug solubility and electronegativity, and further modified via additives such as polyethylene glycol or acid-terminated polyglycolide microdispersions.

### Example 42

### Installation and Self-Elimination of Drug Eluting Fabric in the Bladder

Drug-loaded degradable fabrics from the previous example are installed in the bladder via a rigid 15 Fr scope (Richard Wolf Flexible Fiber-Urethro-Cystoscope), having a working channel of 2.5 mm diameter. First, a barbed suture (4/0 V-LOC, Medronic) is passed through the drug eluting fabric in a single loop along one edge and knotted at the distal end of the suture away from the needle to secure. The suture is selected with a curved non-cutting ¼ circle needle, to support attachment. The sutured fabric is passed through the cystoscope by first curling into a tube-like shape and introducing to the working channel of the scope. A pusher is used to convey the sutured drug-eluting fabric into the bladder, and a needle holder assists in passing the suture through the cystoscope into the bladder. The suture needle and suture is passed through a small thickness of the bladder wall and pulled through to create a knotless secure hold of the drug-eluting fabric in the bladder, taking care to minimize the depth of needle penetration. In the case of chitosan fabrics, there is an additional benefit of natural mucoadhesion to the bladder wall.

The durability and stability of the implant is based on two factors, including the mechanical degradation profile of the fabric as well as degradation profile of the suture attachment. For example, a polyglycolide-base suture is selected for desired duration of about 1-3 weeks, while a polydioxnone-based suture is suitable for 3-5 week implant duration. Upon mechanical degradation of the fabric and/or suture, the material is readily self-removed and excreted from the bladder with urination.

The durability and stability of the implant is based on two factors, including the mechanical degradation profile of the fabric as well as degradation profile of the suture attachment. For example, a polyglycolide-base suture is selected for desired duration of about 1-3 weeks, while a polydioxnone-based suture is suitable for 3-5 week implant duration. Upon mechanical degradation of the fabric and/or suture, the material is readily self-removed and excreted from the bladder with urination.

### Example 43

### Drug Eluting Tubular Stent Structures for Local Delivery into the Bladder

OC9 (Poly-Med, Inc.) was used as a gel-forming, urethane-linked degradable copolymer comprising repeat units of 70% d,l-lactide, 17% polyethylene glycol, and 13% glycolide. First, OC9 was heated at 50° C. for 3 hours to improve miscibility and blended with PEG 400 (Sigma Aldrich) at 2:1 ratio to reduce gel viscosity to assist in tube loading. The OC9/PEG 400 mixture was divided. In one formulation, Triamcinolone acetonide (98+%, Alfa Aesar) was added and mixed with a dual-axis high speed rotary mixer to uniformly incorporate. In a separate formulation, Mitomycin C (MedChemExpress) was added and mix with a dual-axis high speed rotary mixer to uniformly incorporate. Triamcinolone- and Mitomycin C-loaded formulations were transferred into a syringe to aid in part formation.

Stent structures with sizing and materials appropriate for indwelling in the bladder, with variations of materials, porosity, and size. A segment of a Uriprene© Degradable Temporary Ureter Stent (Poly-Med, Inc.) was used as a small diameter degradable stent having a coated textile-based wall with a thin occlusive coating. A small section of a polyethylene (PE) tube was used as an occlusive sheath with a larger core volume. A section of a vascular graft made from a porous synthetic vascular graft, exhibiting the largest diameter and greatest porosity. From previous work it is known that a minimum segment size that will reliably indwell within the bladder is about 5 cm in length. The tubular structures were sized to approximate this length and injected with the drug-loaded OC9-based formulations above according to the associated table. The entire cavity was filled with the respective formulations, and ends closed to prevent leakage.

PDLG (Purasorb PDLG 7507, Corbion) was dissolved in dichloromethane at 15% concentration. Dexamethasone (Sigma Aldrich) was to the solution added at a concentration of 29 mg per gram of PDLG and dissolved. The solution was cast into a thin film using a 25-mil film casting blade and allowed to dry. This film was cut into a strip 1.4 cm in width and rolled around a 1 mm diameter stainless steel mandrel. This form was gently heated to 50° C., cooled to room temperature, and removed from the mandrel to form the hollow tubular implant as described.

TABLE 4
Drug Eluting Tubular and Filled Stent Structures Prepared for Local Bladder
Delivery and which are capable of positioning in a subject via segment length

| | Active Agent, | | | |
|---|---|---|---|---|
| | % Loading, | | | Insertion |
| Form | Location | Sheath | Core | Method |
| 3 mm diameter × | Dexamethasone, | PDLG | None | Introduced |
| 1.4 cm length | 29 mg/g, located | | | via 16F |
| hollow tube, | in sheath | | | Ureteroscope |
| 1 mm wall | | | | with pusher |
| thickness | | | | |
| 2 mm diameter × | Triamcinolone, | Uriprene Degradable | OC9, PEG 400 | Introduced |
| 5.5 cm length, | 10 mg/g, located | Temporary Ureter | | via 16F |
| 0.5 mm wall | in stent core | Stent | | Ureteroscope |
| thickness, | | | | with pusher |
| filled core | | | | |
| 3.9 mm diameter × | Triamcinolone, | PE | OC9, PEG 400 | Introduced |
| 4.5 cm length, | 10 mg/g, located | | | via 16F |
| filled core | in stent core | | | Ureteroscope |
| | | | | with pusher |
| 5 mm diameter × | Triamcinolone, | Porous graft | OC9, PEG 400 | Introduced |
| 5 cm length, | 10 mg/g, located | | | via 16F |
| filled core | in stent core | | | Ureteroscope |
| | | | | with pusher |
| 2 mm diameter × | Mitomycin C, | Uriprene Degradable | OC9, PEG 400 | Introduced |
| 5.5 cm length, | 23 mg/g located | Temporary Ureter | | via 16F |
| 0.5 mm wall | in stent core | Stent | | Ureteroscope |
| thickness, | | | | with pusher |
| filled core | | | | |

The degradable medical device and/or composition has a nominal bladder stability of about 4 weeks, which can be optionally adjusted to less than 2 weeks or about 10 days, up to about 3 months indwelling time in the bladder. PDLG films can remain positionally stable within the bladder for 2 weeks, or optionally up to 3 weeks, or up to 4 weeks before the implant softens, begins to fragment, and is self-excreted with urination. The drug payload is designed to deliver within the span of implant indwelling, which is selected based on the desired therapeutic regimen. Prior to self-elimination, or in the case of non-degradable grafts, installed devices may be retrieved via minimally invasive surgical methods.

### Example 44

### Drug Eluting Flat Encapsulated Films for Local Bladder Drug Delivery and Capable of Positioning within a Subject Because of Size

SVG12 (Poly-Med, Inc.) and PDLG (Purasorb PDLG 7507, Corbion) were separately dissolved in chloroform, each at 15% concentration (wt/vol %). Solutions were divided to create different drug-loading solutions according to the associated table. In short, lidocaine (Sigma Aldrich) was added to a solution of SVG12 at a concentration of 134 mg drug per 1 gram of SVG12. Dexamethasone was added to a separate solution of PDLG at a concentration of 29 mg drug per 1 gram of PDLG. Mitomycin C was added to a separate solution of PDLG at a concentration of 6.8 mg drug per 1 gram of PDLG. All solutions were held at room temperature on a roller until completely dissolved. The solutions were cast into thin films using a 25-mil film casting blade and allowed to dry.

Poly(ethylene vinyl acetate) (EVA, Polysciences) was heated in a glassware oven to melt. Triamcinolone acetonide (98+%, Alfa Aesar) was added to the warm EVA at a ratio of 10 mg drug per 1 gram of EVA. Mixture was reheated to melt EVA and hand mixed a total of 3 times and melt cast into a thin film.

At the same time, SVG12 (Poly-Med, Inc.) was dissolved in dichloromethane at 15% concentration. Lidocaine powder (Sigma Aldrich) was to the solution added at a concentration of 134 mg per gram of SVG12 and dissolved. The solution was cast into a thin film using a 25-mil film casting blade and allowed to dry.

Drug-loaded films were cut into rectangles and placed on SVG12-only films. In the case of the dexamethasone- and lidocaine-loaded sample, the cut films were stacked one on top of the other. In the case of mytomycin C-loaded films, 4 drug-loaded films were stacked one on top of the other to increase total delivery payload. On top of the drug-eluting film, a separate SVG12-only film was added which was larger in size. Degradable medical devices and/or compositions were clamped between rubber films between glass plates, clamped to apply pressure, and heated at 50° C. for 3 hours to create a seal between the encapsulation films. Degradable medical devices and/or compositions were trimmed to size taking care to leave enough of the encapsulation polymer edges to ensure a stable seal.

TABLE 5
Example Drug Eluting Flat Encapsulated Films Prepared for Bladder
Delivery and which are capable of positioning in a subject via size

| | | Drug | Encapsu- | |
|---|---|---|---|---|
| | Active Agent, | containing | lation | Insertion |
| Form | % Loading | Polymer | Polymer | Method |
| 3 cm × 5 cm | Dexamethasone, | PDLG | SVG12 | Introduced |
| Rectangle, | 29 mg/g | | | via 16F |
| 0.5 mm | | | | Ureteroscope |
| thickness | | | | with pusher |
| 3 cm × 3 cm | Lidocaine, | SVG12 | SVG12 | Introduced |
| Rectangle, | 134 mg/g | | | via 16F |
| 0.4 mm | | | | Ureteroscope |
| thickness | | | | with pusher |
| 2.5 cm × 4 cm | Dexamethasone, | PDLG | SVG12 | Introduced |
| Rectangle | 29 mg/g | | | via 16F |
| 0.5 mm | Lidocaine, | SVG12 | | Ureteroscope |
| thickness | 134 mg/g | | | with pusher |
| 4 - 1 cm | Triamcinolone, | EVA | SVG12 | Introduced |
| diameter | 10 mg/g | | | via 16F |
| discs in a | | | | Ureteroscope |
| 5 cm × 5 cm | | | | with pusher |
| rectangle | | | | |
| 0.7 mm | | | | |
| thickness | | | | |
| 2.5 cm × 4 cm | Mitomycin C, | PDLG | SVG12 | Introduced |
| Rectangle | 6.8 mg/g | | | via 16F |
| 0.7 mm | | | | Ureteroscope |
| thickness | | | | with pusher |

### Example 45

### Drug Eluting Films Prepare for Local Bladder Delivery and Positioned Via Integrated Suture Attachment

Drug-incorporated solutions from the previous example were cast into thin films by first placing a suture (Vicryl size 2/0, Ethicon) in a desired conformation on a flat glass plate and clamping to hold. A measure of solution was added along the length of suture to create the desired film shape and size according to the table. The construct was allowed to dry and removed from the glass plate, forming a drug eluting film with an integrated suture anchor.

TABLE 6
Example Drug Eluting Films Prepared for Bladder Delivery
and positioned Via Integrated Suture Attachment

| | | Drug | | |
|---|---|---|---|---|
| | Active Agent, | containing | Attachment | Insertion |
| Form | % Loading | Polymer | Suture | Method |
| 1.3 cm | Lidocaine, | SVG12 | 2/0 Vicryl | Introduced |
| diameter, | 134 mg/g | | | via 16F |
| 0.3 mm | | | | Ureteroscope |
| thickness, | | | | with pusher |
| 3 discs | | | | |
| attached | | | | |
| to suture | | | | |
| 1.3 cm | Dexamethosone, | SVG12 | | |
| diameter, | 29 mg/g | | | |
| 0.3 mm | | | | |
| thickness, | | | | |
| 3 discs | | | | |
| attached | | | | |
| to suture | | | | |
| 2.5 cm × | Lidocaine, | SVG12 | 2/0 Vicryl | Introduced |
| 4 cm, | 134 mg/g | | | via1 6F |
| 0.15 mm | | | | Ureteroscope |
| thickness | | | | with pusher |
| attached | | | | |
| to suture | | | | |

### Example 46

### In Vitro Drug Release Evaluation of Mitomycin C—Eluting Devices in Artificial Urine

Devices prepared from earlier examples were characterized for drug release profile in artificial urine to predict in vivo behavior in a human bladder. First, artificial urine was prepared according to ASTM F1828, with a modified buffer. Drug-containing samples were placed in individual 20 mL glass vials and 15 mL artificial urine added to cover the sample. This volume of artificial urine was sufficiently above the Mitomycin C solubility limit for each sample so as not to interfere with release analysis. Filled glass vials were placed in a 37° C. incubator on a 1 Hz orbital bed. As predetermined time points, a total changeover of artificial urine was performed, with the exchanged urine analyzed by HPLC to determine total release of Mitomycin C per time period.

HPLC analysis was performed using a Waters ACQUITY ArcHPLC with C18 column using an isocratic method with 71:29 water:methanol mobile phase. Peaks were compared against a standard curve to determine concentration in the sample, and calculated to total percentage release based on fluid volume and loading of Mitomycin C in the sample. A gel containing 2 parts OC9 and 1 part PEG 400 released 13% over about 3 days, followed by an extended release plateau. Further attenuation was realized by placing the gel in the core of the stent. A secondary release of drug is anticipated as the OC9 and SVG12 materials experience degradation.

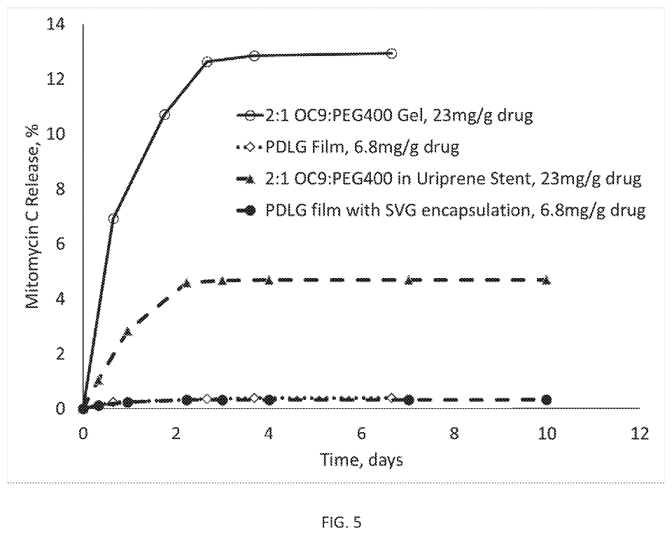

Mitomycin C showed poor stability in PDLG, evidenced by a change in color from purple to tan, highlighting the need for drug/polymer compatibility and use of the polymer to protect the drug from environmental effects. Although any methods and materials similar or equivalent to those described herein can also be used in the practice or testing of the present invention, a limited number of the exemplary methods and materials are described herein. Generally, unless otherwise indicated, the materials for making the invention and/or its components may be selected from appropriate materials such as biodegradable polymers. See FIG. 5 where drug refers to Mitomycin C.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All publications and patents cited herein are incorporated herein by reference in their entirety for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

1. A degradable medical device for delivery of at least one active agent to a urinary bladder, comprising an encapsulating member and at least one active agent-eluting core composition, wherein the encapsulating member, the at least one active agent-eluting core composition, or both are. degradable and when positioned in the bladder are stationary; free-floating; or immobilized by a fastener, wherein the degraded portions are excreted out of the bladder.

2. The degradable device of claim 1, wherein the core composition is encapsulated by a containment layer that at least partially encases the degradable medical device, the degradable medical device being at least partially biodegradable when the medical device is implanted in or provided to a subject.

3. The medical device of claim 2 wherein the containment layer is a coating on at least a portion of the medical device.

4. The medical device of claim 3 wherein the coating is hydrophilic.

5. The medical device of claim 3 wherein the coating is biodegradable, and degrades more slowly than the medical device.

6. The composition of claim 1, further comprising a containment layer that at least partially encapsulates the composition, the composition being at least partially biodegradable when the composition is implanted in a subject, the containment layer being either nonbiodegradable or biodegradable, where the containment layer serves as a container for the composition as that composition degrades in vivo.

7. The composition of claim 6 wherein the containment layer is a coating on the composition.

8. The composition of claim 7 wherein the coating is hydrophilic.

9. The composition of claim 6 wherein the coating is biodegradable, but the coating degrades more slowly than the composition.

10. The medical device of claim 6, wherein the medical device is a single layer film comprising at least active agent, a multi-layer film construct wherein an inner layer comprises at least one active agent and the two opposed outer layers do not comprise an active agent; an indwelling catheter, an indwelling stent; a three-dimensionally printed capsule comprising at least one active agent and a coating; a nonwoven polymeric matrix comprising at least one active agent; an encapsulated delivery bladder; an annular ring or a coil which is hollow; or a nonwoven fabric.

11. A method of local administration of an active agent, comprising providing to a body structure the device of claim 1, wherein the active agent is dispersed from the medical device locally.

12. A method of treatment of bladder cancer comprising
a) administering at least one of a disclosed degradable medical device and/or a composition comprising an effective amount of at least one active agent to the bladder of a subject having bladder cancer, or a subject diagnosed with bladder cancer, or a subject previously treated for bladder cancer; and
b) treating the bladder cancer with the at least one active agent.
